(19) United States
(12) Patent Application Publication
(10) Pub. No.: US 2023/0003720 A1
(43) Pub. Date: Jan. 5, 2023

(54) SYSTEMS FOR DETECTION

(71) Applicant: Koniku Inc., Berkeley, CA (US)

(72) Inventors: Jean-Charles Neel, San Francisco, CA (US); Michael Siani-Rose, San Francisco, CA (US); Oshiorenoya Agabi, Dublin, CA (US)

(21) Appl. No.: 16/344,791

(22) PCT Filed: Oct. 27, 2017
(86) PCT No.: PCT/US2017/058895

§ 371 Date: Apr. 24, 2019

Related U.S. Application Data
(60) Provisional application No. 62/413,897, filed on Oct. 27, 2016.

(51) Int. Cl. G01N 33/50; G01N 33/543; G01N 33/94

(52) U.S. Cl. CPC G01N 33/5058; G01N 33/94; G01N 33/5438

(57) ABSTRACT

Provided herein are arrays, devices, systems, and methods for detection of compounds, such as odorant compounds. The arrays, devices, systems, and methods as described herein may provide patterns of electrical signals, wherein a given pattern may be associated with a given compound or a mixture of compounds such that a presence or a likelihood of a presence of the given compound or mixture of compounds can be determined. The cells expressing an odorant receptor can be present in an array of chambers, each chamber comprising a cell modified to express a unique odorant receptor profile and an electrical component configured to measure an electrical signal in the cell.

### CROSS-REFERENCE

This application claims priority to U.S. Provisional application 62/413,897, filed on Oct. 27, 2016, which is entirely incorporated herein by reference.

### BACKGROUND

Cellular arrays are of utility in medical research and life sciences in general. Traditional cellular arrays use simple containers such as a petri dish or multi-well plate as a vessel for cell culture. However, it is recognized in the art that such a simple approach provides cells with a substantially different environment to that experienced by cells in vivo.

### SUMMARY

An aspect of the present disclosure provides a system. In some embodiments, the system may comprise an array, at least one electrode positioned within each chamber of the plurality of chambers to form a plurality of electrodes configured to measure electrical signals; and a controller configured to receive the electrical signals measured by the plurality of electrodes. In some embodiments, the army may comprise a plurality of chambers, wherein each of the plurality of chambers may comprise a cell expressing one or more cell-surface receptors. In some embodiments, when a compound is introduced into a chamber of the plurality of chambers and a binding event occurs between one or more of the cell-surface receptors of a cell and the compound, an electrical signal may result in response to the binding. In some embodiments, the controller may generate a pattern of electrical signals associated with the compound.

In some embodiments, the at least one electrode may be configured to measure an electrical signal of a cell housed within a respective chamber. In some embodiments, two or more compounds may be introduced. In some embodiments, the compound may be a volatile compound.

In some embodiments, the pattern of electrical signals may be a compound-specific pattern. In some embodiments, the compound-specific pattern may provide a confirmation of a presence of the compound that is introduced into the chamber. In some embodiments, the pattern of electrical signals may be specific to a collection of compounds. In some embodiments, the pattern of electrical signals may provide a unique fingerprint for identifying a presence of the compound in a sample. In some embodiments, the system may be an odorant-detection system.

In some embodiments, the one or more cell-surface receptors may be odorant receptors. In some embodiments, the cell may be a neuron. In some embodiments, the electrical signals may comprise an action potential. In some embodiments, the electrical signals may comprise an excited signal level that is below a threshold for an action potential. In some embodiments, the electrical signals may comprise a cell membrane depolarization.

In some embodiments, the pattern of electrical signals may comprise a binary pattern. In some embodiments, the pattern of electrical signals may comprise magnitudes of individual electrical signals received from individual electrodes. In some embodiments, the pattern of electrical signals may comprise temporal patterns of electrical signals received from individual electrodes. In some embodiments, the controller may receive the pattern of electrical signals and forms a matrix based on the pattern received. In some embodiments, the controller may store patterns of electrical signals associated with specific compounds in a database.

In some embodiments, the cell may be modified to express one or more cell-surface receptors. In some embodiments, the cell may be genetically modified to express one or more cell-surface receptors. In some embodiments, the cell-surface receptors may be modified cell-surface receptors. In some embodiments, the modified cell-surface receptors may comprise a genetic modification, a methylation modification, a sulfentation modification, a sulfentation modification, a acylation modification, an alkylation modification, a butyrylation modification, a glycosylation modification, a malonylation modification, a hydroxylation modification, an iodination modification, a propanoylation modification, or any combination thereof. In some embodiments, the modified cell-surface receptors may comprise an oxidation modification or reduction modification. In some embodiments, the modified ceil-surface receptors may comprise a carbohydrate addition, a carbohydrate deletion, or a combination thereof. In some embodiments, each cell of the plurality of chambers may express a unique cell-surface receptor.

In some embodiments, the compound may comprise any combination of odorant compounds from Table 2a. In some embodiments, the odorant receptor may comprise any combination of odorant receptors from Table 2b. In some embodiments, the odorant receptor may comprise OR1A1 (Gene ID 8383), MOR106-1 (Gene ID 106581391 or Gene ID 56858), OR51E1 (Gene ID 143503 or Gene ID 100145326 or Gene ID 526623 or Gene ID 100388850 or Gene ID 100328817), OR10J5 (Gene ID 127385 or Gene ID 469542 or Gene ID 508986 or Gene ID 488627), OR51E2 (Gene ID 81285 or Gene ID 466344 or Gene ID 100328820 or Gene ID 717504 or Gene ID 510115 or Gene ID 485238), MOR9-1 (Gene ID 259086), MOR18-1 (Gene ID 259097), MOR272-1 (Gene ID 258937), MOR31-1 (Gene ID 18368), MOR136-1 (Gene ID 258953), or any fragment thereof or any combination thereof. In some embodiments, the odorant receptor may comprise any combination of odorant receptors from Table 3. In some embodiments, the odorant receptor may comprise any combination of odorant receptors from Table 4. In some embodiments, the odorant receptor may comprise at least 2 odorant receptors from of Table 2b. In some embodiments, the odorant receptor may comprise at least 2 odorant receptors from Table 3. In some embodiments, the odorant receptor may comprise at least 2 odorant receptors from Table 4 In some embodiments, the odorant receptor may comprise at least 2 odorant receptors from OR1A1, MOR106-1, OR51E1, OR10J5, OR51E2, MOR9-1, MOR18-1, MOR272-1, MOR31-1, MOR136-1, or any fragment thereof or any combination thereof.

In some embodiments, the pattern of electrical signals may represent a probability of a presence of the compound. In some embodiments, the probability may be at least about 75%. In some embodiments, each chamber of the plurality of chambers may be operatively coupled to a respective cell introduction port by a respective cell introduction passage. In some embodiments, the system further may comprise at least one perfusion channel fluidically coupled to a chamber of the plurality of chambers.

Another aspect of the present disclosure provides a method for detecting a presence or a likelihood of a presence of a compound. In some embodiments, the method may detect a presence of a neurotoxin, a toxin, a volatilized plant component, or any combination thereof. In some embodiments, the volatilized plant component may comprise tobacco, marijuana, tetrahydrocannabinol (THC), nicotine, cocaine, or any combination thereof. In some embodiments, the method may detect a presence of an illegal substance as defined in 42 United States Code § 12210. In some embodiments, the method may detect a presence of a carcinogen. In some embodiments, the method may detect a presence of a chemical weapon. In some embodiments, the chemical weapon may be a mustard gas, a sarin gas, or any combination thereof. In some embodiments, the method may detect a presence of a thyomethane, a hydrocarbon, an oxygen, a carbon dioxide, or any combination thereof.

Another aspect of the present disclosure provides a method for confirming a presence or absence of a compound in the sample. In some embodiments, the method may comprise (a) adding a sample to at least one of the plurality of chambers; (b) measuring one or more electrical signals employing the plurality of electrodes; (c) generating a pattern of electrical signals, (d) comparing the pattern of electrical signals to one or more compound-specific patterns stored in a database of the system; and (e) confirming a presence or absence of a compound in the sample based on the comparing.

In one aspect, provided herein is a device comprising: (a) a spatially addressable array, the spatially addressable array comprising: a plurality of chambers, wherein each chamber of the plurality of chambers comprises: (i) a cell modified to express a unique odorant receptor profile; and (n) an electrical component configured to measure an electrical signal in the cell, (b) a controller configured to (i) receive the measured electrical signals from the plurality of chambers and (i) determine a presence or an absence of one or more compounds based on the measured electrical signals.

In another aspect, provided herein is a method of detecting a presence or an absence of one or more compounds in an environment, the method comprising: (a) placing a spatially addressable array in the environment, wherein the spatially addressable array comprises: a plurality of chambers, wherein each chamber of the plurality of chambers comprises: (i) a cell modified to express a unique odorant receptor profile; and (ii) an electrical component configured to measure an electrical signal in the cell; and (b) detecting the presence or the absence of the one or more compounds based the measured electrical signals from the plurality of chambers.

In another aspect, provided herein is a device comprising: an array, the array comprising a plurality of chambers, wherein each of the plurality of chambers comprises: (i) a cell modified to express a cell receptor having a binding specificity for a compound selected from the group consisting of a neurotoxin, a carcinogen, a chemical weapon, and any combination thereof, and (ii) an electrical component configured to measure one or more signals, wherein the device is configured to detect a presence or an absence of the compound based on one or more signals measured in the army.

In another aspect, provided herein is a method of detecting a presence or an absence of a compound in an environment, the method comprising: (a) placing a device in the environment, wherein the device comprises; an array, the array comprising a plurality of chambers, wherein each of the plurality of chambers comprises: (i) a cell modified to express a cell-surface receptor having a binding specificity for the compound, wherein the compound is selected from the group consisting of a neurotoxin, a carcinogen, a chemical weapon, and any combination thereof, and (ii) an electrical component configured to measure one or more signals; and (b) detecting the presence or the absence of the compound based on one or more signals measured in the device.

In another aspect, provided herein is an array of n different cells, each of which expresses a unique odorant receptor, wherein the array is capable of detecting greater than n different compounds each at a confidence level greater than about 70%.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which

FIG. 1 shows an array of cells on a micro-electrode array (MEA).

FIG. 2 shows a neuron expressing an odorant receptor surrounding an electrode.

FIG. 3 shows a computer control system that is programmed or otherwise configured to implement methods provided herein.

FIG. 4 shows a schematic view of a micro-electrode array (MEA) comprising an array of cells.

FIG. 5 provides a table of compounds matched with odorant receptors and the concentration limit of detection for each compound using the respective odorant receptor to detect.

FIG. 6 shows a range of DNT (CAS #121-14-2) concentrations (1 microMolar (uM) to 1000 (uM)) and respective odorant receptor detection as reported by measured levels of luminescence.

FIG. 7 shows a range of vanillic acid (CAS #121-34-6) concentrations (100 picoMolar (pM) to 1 milliMolar (mM)) and respective odorant receptor detection as reported by measured levels of luminescence.

FIG. 8 shows a range of DNT (CAS #121-14-2) concentrations (100 picoMolar (pM) to 1 milliMolar (mM)) and respective odorant receptor detection as reported by measured levels of luminescence.

FIG. 9 shows an action potential of a neuron recorded using a planar electrode.

FIG. 10 shows an action potential of a neuron recorded using a 3D electrode.

FIG. 11 show a spike train obtained using a 3D electrode.

FIG. 12 shows odorant receptor detection of four different compounds (cocaine, heroine, LSD, and PCP) as measured levels of luminescence (y-axis) for a panel of different odorant receptors types (x-axis).

FIG. 13 shows odorant receptor detection of vanillic acid as measured by levels of luminescence (y-axis) at different concentrations (x-axis) for six different types of odorant receptors (legend).

FIG. 14 shows odorant receptor detection as measured by levels of luminescence (y-axis) for a panel of different types of compounds (x-axis) for mouse odorant receptor (mOR9-1).

FIG. 15 shows an electrical signal comprising a spike burst from a broadly tuned receptor for a high affinity compound.

FIG. 16 shows an electrical signal comprising a spike burst from a broadly tuned receptor for a low affinity compound.

FIG. 17 shows a raster plot of Gaussian noise or baseline noise of the detection device.

FIG. 18 shows a raster plot showing spike bursts for two different narrowly tuned receptors. The first narrowly tuned receptor detects odorant A at 500-100 time and the second narrowly tuned receptor detects odorant B at 1500-2000 time. The x-axis is time. The y-axis is cell number.

FIG. 19 shows a raster plot showing spike bursts for broadly tuned receptors. The x-axis is time. The y-axis is cell number.

FIG. 20 shows a raster plot showing spike bursts for broadly tuned receptors. The x-axis is time. The y-axis is cell number.

FIG. 21 shows results for narrowly tuned receptors with electrical signals directly recorded from the neurons having the narrowly tuned receptors. The top panel shows the raw data in a raster plot. The middle panel shows the experimental conditions (or experimental truth) of time exposure for two different compounds (indicated as B or R). The bottom panel shows the predicted outcome.

FIG. 22 shows results for broadly tuned receptors with electrical signals directly recorded from the neurons having the broadly tuned receptors. The top panel shows the raw data in a raster plot. The middle panel shows the experimental condition (or experimental truth) of the time exposure for two different compounds (indicated as B or R). The bottom panel shows the predicted outcome.

FIG. 23 shows results for broadly tuned receptors with electrical signals recorded from another neuron in communication with the primary neuron having the broadly tuned receptors. The top panel shows the raw data in a raster plot. The middle panel shows the experimental condition (or experimental truth) of the time exposure for two different compounds (indicated as B or R). The bottom panel shows the predicted outcome.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described hemin may be employed.

As used herein, the singular forms “a”, “an”, and “the” include plural references unless the context clearly dictates otherwise. Any reference to “or” herein is intended to encompass “and/or” unless otherwise stated.

As used herein, the term “about” means the referenced numeric indication plus or minus 15% of that referenced numeric indication.

The term “cell” as used herein, generally refers to one or more cells A cell may be obtained or isolated from a subject. A cell may be obtained or isolated from a tissue. A subject may be an animal such as a human, a mouse, a rat, a pig, a dog, a rabbit, a sheep, a horse, a chicken or other. A cell may be a neuron. A neuron may be a central neuron, a peripheral neuron, a sensory neuron, an interneuron, a motor neuron, a multipolar neuron, a bipolar neuron, or a pseudo-unipolar neuron. A cell may be a neuron supporting cell, such as a Schwann cell. A cell may be one of the cells of a blood-brain barrier system A cell may be a cell line, such as a neuronal cell line. A cell may be a primary cell, such as cells obtained from a brain of a subject. A cell may be a population of cells that may be isolated from a subject, such as a tissue biopsy, a cytology specimen, a blood sample, a fine needle aspirate (FNA) sample, or any combination thereof. A cell may be obtained from a bodily fluid such as urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid. A cell may comprise cancerous cells, non-cancerous cells, tumor cells, non-tumor cells, healthy cells, or any combination thereof. A cell may be a modified cell, such as a genetically modified cell. A modified cell may comprise an addition of one of more cell-surface receptors, such as modified cell-surface receptors. The modified cell-surface receptors may be modified to increase or decrease their ability to bind to a large set of compounds, a small set of compounds, or a specific compound. A modified cell may comprise a deletion of one or more cell-surface receptors.

The term “tissue” as used herein, generally refers to any tissue sample. A tissue may be a sample suspected or confirmed of having a disease or condition. A tissue may be a sample that is genetically modified. A tissue may be a sample that is healthy, benign, or otherwise free of a disease. A tissue may be a sample removed from a subject, such as a tissue biopsy, a tissue resection, an aspirate (such as a fine needle aspirate), a tissue washing a cytology specimen, a bodily fluid, or any combination thereof. A tissue may comprise cancerous cells, tumor cells, non-cancerous cells, or a combination thereof. A tissue may comprise neurons. A tissue may comprise brain tissue, spinal tissue, or a combination thereof. A tissue may comprise cells representative of a blood-brain barrier. A tissue may comprise a breast tissue, bladder tissue, kidney tissue, liver tissue, colon tissue, thyroid tissue, cervical tissue, prostate tissue, king tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “receptor” as used herein, generally refers to a receptor of a cell. The receptor may be a cell-surface receptor. A cell-surface receptor may be a G coupled protein receptor. A receptor may bind to one or more compounds. A receptor may have a different binding affinity to for each compound to which it binds. A receptor may be modified, such as genetically modified. A receptor may be modified to change the number of compounds to which it may bind. A receptor may be modified to increase the number of different compounds to which it may bind. A receptor may be modified to decrease the number of different compounds to which it may bind. A receptor may bind 1 compound. A receptor may bind 2, 3, 4, 5, 6, 7, 8, 9, 10, 15, 20, 30, 40, 50, 60, 70, 80, 90, 100 compounds or more A receptor may bind less than 10 compounds. A receptor may bind less than 5 compounds. A receptor may bind at least 5 compounds. A receptor may bind at least 10 compounds. A receptor may bind at least 20 compounds. A receptor may be any receptor or combination of receptors of the receptors listed in Table 2b, Table 3, Table 4. A receptor may be any receptor listed in Table 2b, Table 3, Table 4, or any combination thereof that further comprises a modification.

The term “modification” as used herein, generally refers to a modification to a cell or cell receptor. A modification to a cell may include adding one or more receptors, such as modified receptors, to the cell A modification to a cell may include removing one or more receptors from a cell. A modification to a cell may include modifying one or more receptors that are expressed on the cell. A modification to a cell receptor may include a genetic modification. A modification to a cell receptor may include a post-translational modification such as an acylation modification, an acetylation modification, a formylation modification, an alkylation modification, a methylation modification, an arginylation modification, a polyglutamylation modification, a polyglycylation modification, a butyrylation modification, a gamma-carboxylation modification, a glycosylation modification, a malonylation modification, a hydroxylation modification, an iodination modification, a nucleotide addition modification, an oxidation modification, a phosphate ester modification, a propanoylation modification, a pyroglutamate formation modification, an S-glutathionylation modification, an S-nitrosylation modification, an S-sulfenylation modification, a succinylation modification, a sulfation modification, a glycation modification, a carbamylation modification, a carbonylation modification, a biotinylation modification, a pegylation modification, or any combination thereof.

The term “compound” as used herein, generally refers to a composition that may produce a signal in a cell, such as an electrical signal. A compound may comprise an odorant. A compound may comprise a compound that binds an odorant receptor or a modified odorant receptor. A compound may comprise a volatile compound. A compound may comprise an organic volatile compound. A compound may comprise a neurotoxin or a toxin. A compound may comprise any compound or mixture thereof the odorant of Table 2a. A compound may comprise a carcinogen. A compound may comprise a chemical weapon, such as a mustard gas, a sarin gas, or a combination thereof. A compound may comprise an illegal substance as defined in 42 United States Code § 12210. A compound may comprise a drug or a pharmaceutical composition or salt thereof. A compound may comprise a protein, a peptide, a nucleic acid, an antibody, an aptamer, a small molecule. A compound may comprise a cell or a cellular fragment. A compound may comprise a tissue or tissue fragment A compound may comprise a naturally-derived composition or a synthetic composition A compound may be an explosive compound, such as trinitrotoluene (TNT). A compound may be a precursor to the compound (such as a chemical precursor), a degradation product of the compound, or a metabolite of the compound, or any combination thereof. A compound may be any compound described herein, including DNT, RDX, TNT, vanillic acid, or others.

The term “sample” as used herein, generally refers to a sample that may or may not comprise one or more compounds. A sample may be tissue or fluid sample obtained from a subject, such as a human subject. A sample may be a fluid or gas sample obtained from an air space, such as an air space adjacent to a deployment of a chemical weapon or an air space in a residential or commercial setting. A sample may be a blood sample obtained from a subject. A sample may be a soil sample, such as a sample obtained near a fracking system or oil rig system. A sample may be a sample that may comprise a compound that is an environmental hazard or a health hazard. A sample may be a liquid sample obtained from a water system, such as a river, a stream, a lake, an ocean, or others. A sample may be a food sample or a container system that houses a food sample. A pattern or fingerprint of the systems described herein, may confirm a ripeness of a single piece of food, such as a fruit, or a set of fruit.

The term “signal” as used herein, generally refers to a signal in response to a binding event, for example, a compound binding to a cell-surface receptor of a cell. The signal may be an electrical signal. The signal may be a change in a cell membrane potential. The signal may be a membrane depolarization. The signal may be an action potential. The signal may be an electrical signal that is subthreshold of an action potential. The signal may be a magnitude of a change in a cell membrane potential, or a magnitude of an action potential. The signal may be the number of action potentials or a train of action potentials. The signal may be a signal measured over a period of time. Information from a signal may be imported into a matrix to form a fingerprint or a pattern of signals. The fingerprint or pattern of signals may be a unique fingerprint. The signal may be a measurement of a amplitude, a period, or a frequency, of a combination thereof of an electrical signal. The signal may be a time length of a refractory period following an action potential. The signal may be a peak voltage of an action potential. The signal may be a time to a peak voltage of an action potential. The signal may be a peak voltage of a membrane depolarization.

The term “surface roughness” as used herein, generally refers to surface texture or to an amplitude and/or a frequency of deviations on a surface. The deviations may be protrusions and/or recesses. The deviations may form a regular pattern or may be random.

Sensitivity may refer to TP/(TP+FN), where TP may be true positive (correctly detecting a presence of a compound in an environment or sample) and FN may be false negative (incorrectly detecting an absence of a compound in an environment or sample). An array may detect a presence or an absence of one or more compounds at a sensitivity of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds. In some cases, increasing the number of unique odorant receptors within an array may increase the sensitivity of detection for one or more compounds.

Specificity may refer to TN/(TN+FP), where TN may be true negative (correctly detecting an absence of a compound in an environment or sample) and FP may be false positive (incorrectly detecting a presence of a compound in an environment or sample). An array may detect a presence or an absence of one or more compounds at a specificity of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds. Increasing the number of unique odorant receptors within an array may increase the specificity of detection for one or more compounds.

Positive Predictive Value (PPV) may refer to TP/(TP+FP). A PPV may be the proportion of samples with positive test results that correctly detect a presence or an absence of a compound. An array may detect a presence or an absence of one or more compounds at a PPV of greater than about: 70%, 75%, 80%, 85%, 86%, 87%, 88%, 89%. 90%, 91%, 92%, 93%, 94%, 95%, 96%, 97%, 98%, 99%, or 99.5% for the one or more compounds.

Negative Predictive Value (NPV) may refer to TN/(TN+FN). An array may detect a presence or an absence of one or more compounds at an NPV of greater than about: 70%, 75%, 80%, 85%. 86%, 87%, 88%. 890, 90%, 91%, 92%, 93%, 94%, 95%, 96%, 97%, 98%, 90%, or 99.5% for the one or more compounds.

An army as described herein may detect a presence or an absence of one or more compounds at an accuracy of greater than about: 70%, 75%, 80%, 85%, 86%, 87%, 88%, 89%, 90%, 910%, 92%, 93%, 04%, 95%, 96%, 97%, 98%, 99%, or 99.5% for the one or more compounds.

An array as described herein may detect a presence or an absence absence of one or more compounds at a confidence level of greater than about: 70%, 75%, 80%, 85%, 86° %, 87%, 88%, 89%, 90%, 91%, 92%, 93%, 94%, 95%, 96%, 97%, 98%, 99%, or 99.5% for the one or more compounds.

An array as described herein may detect a presence or an absence of one or more compounds at one or more of a sensitivity, a specificity, a PPV, an NPV, an accuracy, a confidence level, or any combination thereof at greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

A device as described herein may be placed into an environment. The environment may be a residential setting such as an indoor or outdoor residential setting. The environment may be a public space. The public space may include an internal public environment such as a public building or may include a public outside space. The environment may be a privately owned space such as a privately owned indoor space (such as a building) or a privately owned outdoor space. A device as described herein may be configured to receive a sample collected from an environment, such as a residential setting or a public space. A sample obtained from an environment may be added to at least a portion of the device. A sample from an environment may contact at least a portion of the device when the device may be placed into the environment.

An array may comprise a unique receptor, such as an odorant receptor. An array may comprise two or more unique receptor profiles. An array may comprise three or more unique receptor profiles. An array may comprise four or more unique receptor profiles. An array may comprise five or more unique receptor profiles. An array may comprise six or more unique receptor profiles. An array may comprise 2, 3, 4, 5, 6, 7, 8, 9, 10, 11, 12, 13, 14, 15, 16, 17, 18, 19, 20 or more unique receptor profiles. An array may comprise from 1-10 unique receptor profiles. An array may comprise from 1-20 unique receptor profiles. An array may comprise from 1-50 unique receptor profiles. An array may comprise from 1-100 unique receptor profiles. An array may comprise from 5-20 unique receptor profiles. A unique receptor may be an odorant receptor. A unique receptor may be a mouse receptor. A unique receptor may be a human receptor. A unique receptor may be an insect receptor.

A device may be configured to detect a presence or an absence of a compound. A device may be configured to detect a presence or an absence of a subset of structurally related compounds. A device may be configured to detect a presence or an absence of a subset of compounds related by function or use (such as explosive compounds or drug compounds). Detection for each compound of a subset may include a confidence level, accuracy, sensitivity, specificity, PPV, NPV or a combination thereof greater that about: 80%, 85%, 90%, 95%, 99% or more.

A device may be configured to detect a presence or an absence of a panel of unique compounds. A device may be configured to detect a presence or an absence of at least two unique compounds. A device may be configured to detect a presence or an absence of at least three unique compounds. A device may be configured to detect a presence or an absence of at least four unique compounds. A device may be configured to detect a presence or an absence of at least five unique compounds. A device may be configured to detect a presence or an absence of at least six unique compounds. A device may be configured to detect a presence or an absence of at least 2, 3, 4, 5, 6, 7, 8, 9, 10, 11, 12, 13, 14, 15, 16, 17, 18, 19, 20 or more unique compounds. A device may be configured to detect a presence or an absence of from 1-10 unique compounds. A device may be configured to detect a presence or an absence of from 1-20 unique compounds. A device may be configured to detect a presence or an absence of from 1-50 unique compounds. A device may be configured to detect a presence or an absence of from 1-100 unique compounds. A device may be configured to detect a presence or an absence of from 5-20 unique compounds.

A cell may be capable of detecting a presence or an absence of more than one unique compound. For example, a cell may comprise a receptor that may be capable of detecting more than one unique compound, such as a broadly tuned receptor. In another example, a cell may comprise two unique receptors, each of which may be capable of detecting a unique compound, such as two narrowly tuned receptors for each of the unique compounds being detected. A cell may be capable of detecting a presence or an absence of 2, 3, 4, 5, 6, 7, 8, 9, 10, 11, 12, 13, 14, 15, 16, 17, 18, 19, 20 or more unique compounds. A cell may be capable of detecting a presence or an absence of from 1-10 unique compounds A cell may be capable of detecting a presence or an absence of from 1-20 unique compounds. A cell may be capable of detecting a presence or an absence of from 1-50 unique compounds. A cell may be capable of detecting a presence or an absence of from 1-100 unique compounds. A cell may be capable of detecting a presence or an absence of from 5-20 unique compounds.

A cell may be modified to express a receptor. The receptor may be an odorant receptor. The receptor may be a wild-type receptor. The receptor may be a modified receptor, such as a genetically modified receptor. A receptor may be modified to enhance a binding specificity to a particular compound or to alter the receptor from a broadly tuned receptor to a narrowly tuned receptor or vice versus. The cell may be modified to express more than one unique receptor. The cell may be modified to express two unique receptors. The cell may be modified to express three or more unique receptors. A receptor may be a human receptor, a mouse receptor, an insect receptor, or other species type of odorant receptor.

A cell may be modified to express one or more of OR1A1 (Homo sapiens), mOR106-1 (Mus musculus), OR51E1 (Homo sapiens), OR10J5 (Homo sapiens), OR51E2 (Homo sapiens), mOR9-1 (Mus musculus), mOR18-1 (Mus musculus), mOR272-1 (Mus musculus), mOR31-1 (Mus musculus), mOR136-1 (Mus musculus), any genetic variation thereof, any functionally active fragment thereof or any combination thereof. An array may comprise one or more of OR1A1 (Homo sapiens), mOR106-1 (Mus musculus), OR51E1 (Homo sapiens), OR10J5 (Homo sapiens), OR51E2 (Homo sapiens), mOR9-1 (Mus musculus), mOR18-1 (Mus musculus), mOR272-1 (Mus musculus), mOR31-1 (Mus musculus), mOR136-1 (Mus musculus), any genetic variation thereof any functionally active fragment thereof, or any combination thereof.

A cell may be modified to express one or more of mOR185-1, mOR189-1, mOR9-1, mOR162-2, mOR202-37, mOR204-3, mOR204-8, mOR204-11, mOR188-1, mOR256-3, any genetic variation thereof any human analog thereof any functionally active fragment thereof or any combination thereof. An array may comprise one or more of mOR185-1, mOR189-1, mOR9-1, mOR162-2, mOR202-37, mOR204-3, mOR204-8, mOR204-11, mOR188-1, mOR256-3, any genetic variation thereof any human analog thereof any functionally active fragment thereof or any combination thereof.

A cell may be modified to express one or more of 156-5, 159-3, 160-4, 160-5, 161-5, 161-6, 162-2, 164-2, 202-37 204-3, 204-8, 204-11, any genetic variation thereof any human analog thereof, any functionally active fragment thereof or any combination thereof. An array may comprise one or more of 156-5, 159-3, 160-4, 160-5, 161-5, 161-6, 162-2, 164-2, 202-37, 204-3, 204-8, 204-11, any genetic variation thereof any human analog thereof, any functionally active fragment thereof or any combination thereof.

A cell may be modified to express one or more of 165-1, 178-1, 179-1, 183-1, 185-1, 162-1, 189-1, any genetic variation thereof any human analog thereof any functionally active fragment thereof or any combination thereof. An array may comprise one or more of 165-1, 178-1, 179-1, 183-1, 185-1, 162-1, 189-1, any genetic variation thereof any human analog thereof, any functionally active fragment thereof or any combination thereof.

A cell may be modified to express one or more of OR1A1, mOR106-1, OR51E1, OR10J5, OR51E2, mOR9-1, mOR18-1, mOR272-1, mOR31-1, mOR136-1, mOR185-1, mOR189-1, mOR9-1, mOR162-2, mOR202-37, mOR204-3, mOR204-8, mOR204-11, mOR188-1, mOR256-3, mOR156-5, mOR159-3, mOR160-4, mOR160-5, mOR161-5, mOR161-6, mOR162-2, mOR164-2, mOR202-37, mOR204-3, mOR204-8, mOR204-11, mOR165-1, mOR178-1, mOR179-1, mOR183-1, mOR185-1, mOR162-1, mOR189-1, any genetic variation thereof, any human analog thereof any functionally active fragment thereof or any combination thereof. An array may comprise one or more of OR1A1, mOR106-1, OR51E1, OR10J5, OR51E2, mOR9-1, mOR18-1, mOR272-1, mOR31-1, mOR136-1, mOR185-1, mOR189-1, mOR9-1, mOR162-2, mOR202-37, mOR204-3, mOR204-8, mOR204-11, mOR188-1, mOR256-3, mOR156-5, mOR159-3, mOR160-4, mOR160-5, mOR161-5, mOR161-6, mOR162-2, mOR164-2, mOR202-37, mOR204-3, mOR204-8, mOR204-11, mOR165-1, mOR178-1, mOR179-1, mOR183-1, mOR185-1, mOR162-1, mOR189-1, any genetic variation thereof any human analog thereof any functionally active fragment thereof or any combination thereof.

A cell may be modified to express one or more of OR1A1, mOR106-1, OR51E1, OR10J5, OR51E2, mOR9-1, mOR18-1, mOR272-1, mOR31-1, mOR136-1, mOR185-1, mOR189-1, mOR9-1, mOR162-2, mOR202-37, mOR204-3, mOR204-8, mOR204-11, mOR188-1, mOR256-3, mOR156-5, mOR159-3, mOR160-4, mOR160-5, mOR161-5, mOR161-6, mOR162-2, mOR164-2, mOR202-37, mOR204-3, mOR204-8, mOR204-11, mOR165-1, mOR178-1, mOR179-1, mOR183-1, mOR185-1, mOR162-1, mOR189-1, any genetic variation thereof any human analog thereof, any functionally active fragment thereof or any combination thereof. An array may comprise one or more of OR1A1, mOR106-1, OR51E1, OR10J5, OR51E2, mOR9-1, mOR18-1, mOR272-1, mOR31-1, mOR136-1, mOR185-1, mOR189-1, mOR9-1, mOR162-2, mOR202-37, mOR204-3, mOR204-8, mOR204-11, mOR188-1, mOR256-3, mOR156-5, mOR159-3, mOR160-4, mOR160-5, mOR161-5, mOR161-6, mOR162-2, mOR164-2, mOR202-37, mOR204-3, mOR204-8, mOR204-11, mOR165-1, mOR178-1, mOR179-1, mOR183-1, mOR185-1, mOR162-1, mOR189-1, any genetic variation thereof, any human analog thereof; any functionally active fragment thereof; or any combination thereof.

A cell may be modified to express one or more receptors of Table 2b, Table 3, Table 4, any genetic variation thereof any human analog thereof any functionally active fragment thereof or any combination thereof. An array may comprise one or more receptors of Table 2b, Table 3, Table 4, any genetic variation thereof, any human analog thereof, any functionally active fragment thereof, or any combination thereof.

A device may communicate a result via a communication medium, such as a printed report, a computer screen, a user interface, a portable electronic device such as a phone, an iPad, laptop, or other portable device, or any combination thereof. A result may comprise a probability of a presence or an absence of a compound. The probability may be at least about: 70%, 75%, 80%, 81%, 82%, 83%, 84%, 85%, 86%, 87%, 88%, 89%, 90%, 91%, 92%, 93%, 94%, 95%, 96%, 97%, 98% or 99%. The probability may be from about 80% to about 99%. The probability may be from about 70% to about 99%. The probability may be from about 75% to about 99%6. The probability may be from about 85% to about 99%. The probability may be from about 90% to about 99%. The probability may be from about 80% to 100%. The probability may be from about 85% to 100%. The probability may be from about 90% to 100%.

A device may detect a presence or an absence of a compound at a concentration detection limit of about: 10 millimolar (mM), 5 mM, 1 mM, 100 micromolar (uM), 50 uM, 10 uM, 5 uM, 1 uM, 100 nanomolar (nM), 50 nM, 10 nM, 5 nM, 1 nM, 100 picomolar (pM), 50 pM, 10 pM, 5 pM, 1 pM, or less. A concentration detection limit may be 100 uM or less. A concentration detection limit may be 50 uM or less. A concentration detection limit may be 10 uM or less. A concentration detection limit may be 5 uM or less A concentration detection limit may be 1 uM or less. A concentration detection limit may be from about 100 uM to about 1 uM. A concentration detection limit may be from about 50 uM to about 1 uM. A concentration detection limit may be from about 10 uM to about 1 uM. A concentration detection limit may be from about 10 uM to about 100 nM. A concentration detection limit may be compound specific.

A device may store one or more signals or signaling patterns associated with one or more reference compounds in a database. A device may comprise a database having signaling information for at least 2, 3, 4, 5, 6, 7, 8, 9, 10, 20, 30, 40, 50, 60, 70, 80, 90, 100, 200, 500 or more reference compounds. A device may comprise a database having signaling information for from 2 to 10 reference compounds. A device may comprise a database having signaling information for from 2 to 20 reference compounds. A device may comprise a database having signaling information for from 2 to 50 reference compounds. A device may comprise a database having signaling information for from 2 to 100 reference compounds. A device may comprise a database having signaling information for from 2 to 200 reference compounds.

A device as described herein may provide a greater specificity for a given compound, a lower concentration detection threshold for a given compound, or a combination thereof, as compared to a device lacking the spatially addressable array. A signaling pattern may be specific for a given compound. A signaling pattern may be specific for a combination of unique compounds. Each individual compound or combination of uniquid compounds may be a uniquely identifier pattern of signals, such as a fingerprint pattern.

A device as described herein may comprise a specificity for a given compound of greater than about: 70%, 75%, 80%, 85%, 90%, 91%, 92%, 93%, 94%, 95%, 96%, 97%, 98% 99% specificity or greater. A device may comprise a specificity for a given compound of from about 80% to about 99% specificity. A device may comprise a specificity for a given compound of from about 85% to about 99% specificity. A device may comprise a specificity for a given compound of from about 90% to about 99% specificity. A device may comprise a specificity for a given compound of from about 95% to about 99% specificity. A device may comprise a specificity for a given compound of from about 80% to 100% specificity. A device may comprise a specificity for a given compound of from about 85% to 100% specificity.

A device as described herein may comprise a sensitivity for a given compound of greater than about: 70%, 75%, 80%, 85%, 90%, 91%, 92%, 93%, 94%, 95%, 96%, 97%, 98% 99% sensitivity or greater. A device may comprise a sensitivity for a given compound of from about 80% to about 99% sensitivity. A device may comprise a sensitivity for a given compound of from about 85% to about 99% sensitivity. A device may comprise a sensitivity for a given compound of from about 90% to about 99% sensitivity. A device may comprise a sensitivity for a given compound of from about 95% to about 99% sensitivity. A device may comprise a sensitivity for a given compound of from about 80% to 100% sensitivity. A device may comprise a sensitivity for a given compound of from about 85% to 100% sensitivity.

A device as described herein may comprise an accuracy for a given compound of greater than about: 70%, 75%, 80%, 85%, 90%, 91%, 92%, 93%, 94%, 95%, 96%, 97%, 98% 99% accuracy or greater. A device may comprise an accuracy for a given compound of from about 80% to about 99% accuracy. A device may comprise an accuracy for a given compound of from about 85% to about 99% accuracy. A device may comprise an accuracy for a given compound of from about 90% to about 99% accuracy. A device may comprise an accuracy for a given compound of from about 95% to about 99% accuracy. A device may comprise an accuracy for a given compound of from about 80% to 100% accuracy. A device may comprise an accuracy for a given compound of from about 85% to 100% accuracy.

A device as described herein may comprise a concentration detection threshold that may be about: 5%, 10%, 15%, 20%, 25%, 30%, 35% lower than a device lacking a spatially addressable array or a device lacking two or more unique receptor profiles A device may comprise a concentration detection threshold that may be from about 5% to about 35% lower than a device lacking a spatially addressable array or a device lacking two or more unique receptor profiles. A device may comprise a concentration detection threshold that may be from about 5% to about 20% lower than a device lacking a spatially addressable array or a device lacking two or more unique receptor profiles. A device may comprise a concentration detection threshold that may be from about 3% to about 10% lower than a device lacking a spatially addressable array or two or more unique receptor profiles.

A device may be modified to enhance a specificity, a concentration detection threshold, or a combination thereof for one or more compounds. For example, a device may be modified to enhance a specificity for Compounds A and B of a panel of compounds that the device may be capable of detecting. A device may be modified to enhance a specificity within a specific range, such as from 80% to 99% specificity. A device may be modified by altering the panel of unique receptors within the device. A device may be modified by altering a receptor profile of a given cell. A device may be modified by selecting for receptors having greater binding specificity for the given compound of interest. A device may be modified by selecting for a receptor having a sequence modification to enhance binding specificity for a given compound of interest.

A device may comprise one or more unique receptor profiles. For example, a device may comprise 1, 2, 3, 4, 5, 6, 7, 8, 9, 10, 15, 20, 50 or more unique receptor profiles. A device may comprise receptors that may be broadly tuned to a particular compound and receptors that may be narrowly tuned to a particular compound. For example, a broadly tuned receptor may bind to a particular compound with a lower specificity than a narrowly tuned receptor. For example, a broadly tuned receptor may bind to a greater number of off-target compounds in addition to the particular compound of interest as compared to the lower number of off-target compounds that bind to a narrowly tuned receptor. A device may comprise a panel of receptors, each receptor having a different tuning for a particular compound.

A benefit of incorporating broad and narrow tuned receptors into an array may be (i) an increased specificity, an increased sensitivity, an increased confidence level, an increased accuracy, an increased PPV or NPV for diagnosing a presence or an absence of a particular compound in an environment, (ii) a lower concentration threshold for detection of one or more compounds, or (iii) a combination thereof. An electrical signal obtained from cell having a narrowly tuned receptor may be unique and distinguishable from an electrical signal obtained from a cell having a broadly tuned receptor. An array having a combination of broadly tuned and narrowly tuned receptors for a given compound may provide a unique fingerprint of detection (comprising measured electrical signals) for the presence or the absence of the compound in a sample or environment A unique fingerprint for a given compound may be a signaling pattern. A unique fingerprint for a given compound may comprise (i) a spatial pattern of chambers of the array having measured patterns of electrical signals, (i) a pattern of electrical signals measured within a chamber of the array, or (ii) a combination thereof. A unique fingerprint for a given compound may also be specific to the combination of receptor profiles of the array.

### Electrical Components

An electrical component may comprise an electrode. An electrode may be a two-dimensional electrode. An electrode may be a three-dimensional electrode. An electrical component may comprise one or more sensors, such as a temperature sensor, a pH sensor, a gas sensor, a glucose sensor, a level sensor, or any combination thereof. In some embodiments, the gas sensor may be an O₂ sensor, a CO₂ sensor, or a combination thereof. In some embodiments, the one or more sensors may comprise an optical sensor, an electrochemical sensor, an opto-electric sensor, a piezoelectric sensor, a biosensor, or any combination thereof. In some embodiments, each chamber of the plurality of chambers may comprise at least one sensor. In some embodiments, the device may further comprise a controller configured to instruct the electrical components to collect one or more measurements respective to the one or more sensors.

An electrode may comprise a metal. An electrode may comprise an alloy. An electrode may comprise aluminum, gold, lithium, copper, graphite, carbon, titanium, brass, silver, platinum, palladium, cesium carbonate, molybdenum(VI) oxide, or any combination thereof. An electrode may comprise a mixed metal oxide.

Modifying an electrode with a plurality of protrusions, a plurality of recesses, modifying by adding a surface roughness to the surface of an electrode may increase the surface area of the electrode. This modification or enhanced surface area may enhance the amount of cellular attachment to the electrode. This modification may enhance the portion of the electrode that is contacted or at least partially engulfed by a cell. This modification may enhance the portion of the electrode that is contacted by a cell. This modification may enhance an electrical connection between a cell and an electrode.

An electrode may comprise a spherical shape, a hemispherical shape, a mushroom shape, comprising a head portion and support portion, a rod-like shape, a cylindrical shape, a conical shape, a patch shape, or any combination thereof. A cell culture module may comprise electrodes having the same shape. For example, a module may comprise 10 electrodes of a mushroom shape. A cell culture module may comprise electrodes of more than one type of shape. For example, a module may comprise 10 electrodes of a mushroom shape and 10 electrodes of a conical shape.

An electrode may have a combination of one or more protrusions and one or more recesses. An electrode may have a combination of one or more protrusion shapes, such as a hemispherical protrusion, and one or more recess shapes, such as a hemispherical recess.

A protrusion may be a hemispherical protrusion. A protrusion may be a spike protrusion, a conical protrusion, a square or rectangular rod protrusion, an obelisk protrusion, a cylindrical protrusion, a hemispherical protrusion, or any combination thereof. A recess may be a hemispherical recess. A recess may be a V-groove recess, a dovetail recess, a spike recess, a conical recess, a cylindrical recess, a square or rectangular rod recess, a hemispherical recess, or any combination thereof.

An electrode may have one or more protrusions. An electrode may have 10 protrusions. An electrode may have at least 10 protrusions. An electrode may have 20 protrusions. An electrode may have at least 20 protrusions. An electrode may have 100 protrusions. An electrode may have at least 100 protrusions. An electrode may have 500 protrusions. An electrode may have at least 500 portions. An electrode may have 1000 protrusions. An electrode may have at least 1000 protrusions. An electrode may have 2000 protrusions. An electrode may have at least 2000 protrusions. An electrode may have 2, 3, 4, 5, 6, 7, 8, 9, 10, 15, 20, 25, 30, 35, 40, 45, 50, 100, 150, 200, 250, 500, 750, 1000, 1500, 2000, 3000, 4000, 5000 protrusions or more.

An electrode may have a surface concentration of protrusions of about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10 protrusions per micrometer squared (pro/um²). An electrode may have a surface concentration of protrusions of about 0.0001 pro/um². An electrode may have a surface concentration of protrusions of about 0.001 pro/un. An electrode may have a surface concentration of protrusions of about 0.01 pro/um². An electrode may have a surface concentration of protrusions of about 0.1 pro/um². An electrode may have a surface concentration of protrusions of about 0.5 pro/um². An electrode may have a surface concentration of protrusions from about 0.0001 to about 0.01 pro/um². An electrode may have a surface concentration of protrusions from about 0.001 to about 0.01 pro/um². An electrode may have a surface concentration of protrusions from about 0.001 to about 0.1 pro/um². An electrode may have a surface concentration of protrusions from about 0.0005 to about 0.5 pro/um². An electrode may have a surface concentration of protrusions from about 0.05 to about 5 pro/um².

An electrode may have one or more recesses. An electrode may have 10 recesses. An electrode may have at least 10 recesses. An electrode may have 20 recesses. An electrode may have at least 20 recesses. An electrode may have 100 recesses. An electrode may have at least 100 recesses. An electrode may have 500 recesses. An electrode may have at least 500 recesses. An electrode may have 1000 recesses. An electrode may have at least 1000 recesses. An electrode may have 2000 recesses. An electrode may have at least 2000 recesses. An electrode may have 2, 3, 4, 5, 6, 7, 8, 9, 10, 15, 20, 25, 30, 35, 40, 45, 50, 100, 150, 200, 250, 500, 750, 1000, 1500, 2000, 3000, 4000, 5000 recesses or more.

An electrode may have a surface concentration of recesses of about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10 recesses per micrometer squared (rec/um²). An electrode may have a surface concentration of recesses of about 0.0001 rec/um². An electrode may have a surface concentration of recesses of about 0.001 rec/um². An electrode may have a surface concentration of recesses of about 0.01 rec/um. An electrode may have a surface concentration of recesses of about 0.1 rec/um². An electrode may have a surface concentration of recesses of about 0.5 rec/um². An electrode may have a surface concentration of recesses from about 0.0001 to about 0.01 rec/um². An electrode may have a surface concentration of recesses from about 0.001 to about 0.01 rec/um². An electrode may have a surface concentration of recesses from about 0.001 to about 0.1 rec/um². An electrode may have a surface concentration of recesses from about 0.0005 to about 0.5 rec/um². An electrode may have a surface concentration of recesses from about 0.05 to about 5 rec/um².

The surface of an electrode may be smooth. The surface of an electrode may have a surface roughness. A surface roughness may be uniform across the surface of an electrode. A portion of the surface of an electrode may have a surface roughness, such as a top portion of the electrode, a bottom portion of the electrode. An electrode may have alternating rows of smooth and rough portions.

A surface roughness may be about 5, 10, 15, 20, 25, 30, 35, 50, 100, 200, 300, 400, 500, 600, 700, 800, 900, 1000, 1500, 2000, 2500, 3000 nanometers (nm) or more. A surface roughness may be from about 5 to about 50 nm. A surface roughness may be from about 5 to about 100 nm. A surface roughness may be from about 5 to about 500 nm. A surface roughness may be from about 10 to about 50 nm. A surface roughness may be from about 10 to about 100 nm. A surface roughness may be from about 10 to about 500 nm.

A width of an electrode may be of a size to accommodate a cell to contact or at least partially engulf the electrode. A width of an electrode may be about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 35, 40, 45, 50 micrometers (um). A width of an electrode may be about 2 um. A width of an electrode may be about 5 um. A width of an electrode may be about 10 um. A width of an electrode may be about 15 um A width of an electrode may be about 20 um A width of an electrode may be greater than 2 um. A width of an electrode may be greater than 3 um. A width of an electrode may be greater than 4 um. A width of an electrode may be greater than 5 um A width of an electrode may be greater than 6 um. A width of an electrode may be greater than 7 um. A width of an electrode may be greater than 8 um. A width of an electrode may be greater than 9 um A width of an electrode may be greater than 10 um. A width of an electrode may be a width of the support portion. A width of an electrode may be a width of the head portion.

### Medium

A medium may include one or more components. A medium may include one or more of sodium chloride, glycine, 1-alanine, 1-serine, a neuroactive inorganic salt, 1-aspartic acid, 1-glutamic acid, or any combination thereof. A medium may further include one or more of a pH modulating agent, amino acid, vitamin, supplemental agent, protein, energetic substrate, light sensitive agent, or any combination thereof. A medium may further include one or more buffering agents. A medium may further include one or more antioxidants.

The sodium chloride may be present in the medium at a concentration of between about 70 mM and about 150 mM. The sodium chloride may be present in the medium at a concentration of between about 50 mM and about 65 mM. The sodium chloride may be present in the medium at a concentration of between about 155 mM and about 200 mM. The sodium chloride may be present in the medium at a concentration of between about 70 mM and about 100 mM. The sodium chloride may be present in the medium at a concentration of between about 100 mM and about 150 mM.

The neuroactive inorganic salt may be present in the medium at a concentration of between about 0.000001 and about 10 mM. The neuroactive inorganic salt may include one or more of potassium chloride, calcium chloride, magnesium sulfate, magnesium chloride, ferric nitrate, zinc sulfate, cupric sulfate, ferric sulfate, or any combination thereof.

The glycine may be present in the medium at a concentration of between about 0.0001 and about 0.05 mM. The glycine may be present in the medium at a concentration of between about 0.06 and about 0.10 mM. The glycine may be present in the medium at a concentration of between about 0.000001 and about 0.00009 mM. The glycine may be present in the medium at a concentration of between about 0.01 and about 0.05 mM. The glycine may be present in the medium at a concentration of between about 0.001 and about 0.005 mM. The glycine may be present in the medium at a concentration of between about 0.0001 and about 0.0005 mM.

The L-alanine may be present in the medium at a concentration of between about 0.00001 and about 0.05 mM. The L-alanine may be present in the medium at a concentration of between about 0.06 and about 0.10 mM. The 1, alanine may be present in the medium at a concentration of between about 0.000001 and about 0.00001 mM. The L-alanine may be present in the medium at a concentration of between about 0.00001 and about 0.0005 mM. The L-alanine may be present in the medium at a concentration of between about 0.01 and about 0.05 mM. The L-alanine may be present in the medium at a concentration of between about 0.0001 and about 0.005 mM.

The L-serine may be present in the medium at a concentration of between about 0.001 and about 0.03 mM The L-serine may be present in the medium at a concentration of between about 0.04 and about 0.10 mM. The L-serine may be present in the medium at a concentration of between about 0.00001 and about 0.0009 mM. The L-serine may be present in the medium at a concentration of between about 0.01 and about 0.03 mM. The L-serine may be present in the medium at a concentration of between about 0.001 and about 0.01 mM.

The L-aspartic acid may be present in the medium at a concentration of between about 0.00001 and about 0.003 mM. The L-aspartic acid may be present in the medium at a concentration of between about 0.0035 and about 0.006 mM. The L-aspartic acid may be present in the medium at a concentration of between about 0.0000001 and about 0.000009 mM. The L-aspartic acid may be present in the medium at a concentration of about 0.0031 mM. The L-aspartic acid may be present in the medium at a concentration of about 0.000009 mM.

The L-glutamic acid may be present in the medium at a concentration of between about 0.00001 and about 0.02 mM. The L-glutamic acid may be present in the medium at a concentration of between about 0.022 and about 0.06 mM. The L-glutamic acid may be present in the medium at a concentration of between about 0.0000001 and about 0.000009 mM. The L-glutamic acid may be present in the medium at a concentration of about 0.022 mM. The L-glutamic acid may be present in the medium at a concentration of about 0.000009 mM.

A medium may include one or more components. For example, a medium may include one or more pH modulating agents. The one or more pH modulating agents may include a buffering agent, an inorganic salt, or any combination thereof. An inorganic salt may include sodium phosphate dibasic, sodium phosphate monobasic, or any combination thereof.

A pH modulating agent (such as an inorganic salt) may be present in the medium at a concentration of between about 0.001 and about 1 mM A pH modulating agent (such as an inorganic salt) may be present in the medium at a concentration of between about 1.1 and about 5 mM. A pH modulating agent (such as an inorganic salt) may be present in the medium at a concentration of between about 0.00001 and about 0.0009 mM.

An inorganic salt (such as sodium bicarbonate) may be present in the medium at a concentration of between about 1 and about 35 mM. An inorganic salt (such as sodium bicarbonate) may be present in the medium at a concentration of between about 36 and about 50 mM. An inorganic salt (such as sodium bicarbonate) may be present in the medium at a concentration of between about 0.001 and about 0.09 mM.

A medium may include one or more components. For example, a medium may include one or more amino acids. The one or more amino acid may include L-Alanyl-L-Glutamine, L-Arginine hydrochloride, L-Asparagine-H2O, L-Cysteine hydrochloride-H2O, L-Cystine 2HCl, L-Histidine hydrochloride-H2O, L-Isoleucine, L-Leucine, L-Lysine hydrochloride, L-Methionine, L-Phenylalanine, L-Proline, L-Threonine, L-Tryptophan, L-Tyrosine disodium salt dihydrate, L-Valine, or any combination thereof. The amino acid may be present in the medium at a concentration of between about 0.001 and about 1 mM. The amino acid may be present in the medium at a concentration of between about 1 and about 5 mM. The amino acid may be present in the medium at a concentration of between about 0.00001 and about 0.0009 mM. The amino acid may be present in the medium at a concentration of between about 0.1 and about 1 mM. The amino acid may be present in the medium at a concentration of between about 0.001 and about 0.1 mM.

A medium may include one or more components. For example, a medium may include one or more vitamins. The one or more vitamins may include choline chloride, D-calcium pantothenate (B5), folic acid (B9), i-Inositol, niacinamide (B3), pyridoxine hydrochloride, thiamine hydrochloride, vitamin B12 (cyanocobalamin), riboflavin (B2), or any combination thereof. The vitamin may be present in the medium at a concentration of between about 0.00001 and about 1 mM. The vitamin may be present in the medium at a concentration of between about 1 and about 5 mM. The vitamin may be present in the medium at a concentration of between about 0.0000001 and about 0.000009 mM. The vitamin may be present in the medium at a concentration of between about 0.001 and about 1 mM. The vitamin may be present in the medium at a concentration of between about 0.00001 and about 0.001 mM.

In some cases, the one or more vitamins may include choline chloride, D-calcium pantothenate (B5), folic acid (B9), i-Inositol, niacinamide (B3), pyridoxine hydrochloride, thiamine hydrochloride, vitamin B 12 (cyanocobalamin), riboflavin (B2), or any combination thereof, the vitamin may be present in the medium at a concentration of between about 0.00001 and about 1 mM, or between about 0.00005 and about 0.5 mM, or between about 0.0001 and about 0.9 mM, or between about 0.0005 and about 0.8 mM, or between about 0.001 and about 0.7 mM, or between about 0.005 and about 0.6 mM, or between about 0.01 and about 0.5 mM, or between about 0.05 and about 0.1 mM.

In some cases, the one or more vitamins may include choline chloride at a concentration of about 0.07 mM, D-calcium pantothenate (B5) at a concentration of about 0.006 mM, folic acid (B9) at a concentration of about 0.006 mM, i-Inositol at a concentration of about 0.07 mM, niacinamide (B3) at a concentration of about 0.02 mM, pyridoxine hydrochloride at a concentration of about 0.010 mM, thiamine hydrochloride at a concentration of about 0.007 mM, vitamin B32 (cyanocobalamin) at a concentration of about 0.0006 mM, riboflavin (132) at a concentration of about 0.0006 mM, and combinations thereof.

In some cases, the one or more vitamins may include choline chloride at a concentration of about 0.05 mM, D-calcium pantothenate (B5) at a concentration of about 0.004 mM, folic acid (139) at a concentration of about 0.004 mM, i-Inositol at a concentration of about 0.05 mM, niacinamide (B3) at a concentration of about 0.01 mM, pyridoxine hydrochloride at a concentration of about 0.008 mM, thiamine hydrochloride at a concentration of about 0.005 mM, vitamin B32 (cyanocobalamin) at a concentration of about 0.0004 mM, riboflavin (B2) at a concentration of about 0.0004 mM, and combinations thereof.

A medium may include one or more components. For example, a medium may include one or more supplemental agents. The one or more supplemental agents may include a protein, a neurotrophic factor, a steroid, a hormone, a fatty acid, a lipid, a vitamin, a sulfate mineral, an organic chemical compound, a monosaccharide, a nucleotide, or any combination thereof.

A supplemental agent may include a protein (for example, laminin, BSA, fatty acid free fraction V; catalase; insulin; human recombinant insulin; insulin recombinant full chain; transferrin; human transferrin; human transferrin (Holo); superoxide dismutase), neurotrophic factor (for example, Human brain derived neurotrophic factor (BDNF); glia neurotrophic factor (GDNF)), hormone, steroid (for example, corticosterone, progesterone), hormone thyroid (for example, T3 (triodo-I-thyronine)), fatty acid, essential fatty acid (for example, linoleic acid, linolenic acid), lipid (for example, cholesterol), vitamin (for example, ascorbic acid (Vit C), biotin (B7), DL alpha tocopherol acetate, DL alpha-tocopherol, vitamin A (retinoic acid)), sulfate mineral (for example, selenite), organic chemical compound (for example, putrescine 2HCl), monosacharide (for example, D-Galactose), nucleotide (for example, dibutyril cAMP sodium salt), or any combination thereof.

The supplemental agent may be present in the medium at a concentration of between about 0.01 and about 50 μg/mL, or between about 1 and about 40 μg/mL, or between about 5 and about 30 μg/mL, or between about 10 and about 20 μg/mL. The supplemental agent may be present in the medium at a concentration of between about 0.01 and about 50 mg/mL, or between about 1 and about 40 mg/mL, or between about 5 and about 30 mg/mL, or between about 10 and about 20 mg/mL. The supplemental agent may be present in the medium at a concentration of between about 0.000001 and about 1 mM, or between about 0.00001 and about 0.5 mM, or between about 0.0001 and about 0.1 mM, or between abut 0.001 and about 0.01 mM.

A medium may include one or more components. For example, a medium may include one or more energetic substrates. The one or more energetic substrates may include a sugar, sodium pyruvate, or a combination thereof. The energetic substrate may be present in the medium at a concentration of between about 0.1 and about 5 mM. The energetic substrate may be present in the medium at a concentration of between about 5 and about 10 mM. The energetic substrate may be present in the medium at a concentration of between about 0.001 and about 0.09 mM. The energetic substrate may be present in the medium at a concentration of between about 1 and about 5 mM. The energetic substrate may be present in the medium at a concentration of between about 0.1 and about 1 mM.

A medium may include one or more components. For example, a medium may include one or more light sensitive agents. The light sensitive agent may include riboflavin (B2), HEPES, or a combination thereof. The light sensitive agent (such as riboflavin (B2)) may be present in the medium at a concentration of between about 0.0001 and about 0.0006 mM. The light sensitive agent (such as riboflavin (B2)) may be present in the medium at a concentration of between about 0.000001 and about 0.00009 mM. The light sensitive agent (such as riboflavin (B2)) may be present in the medium at a concentration of between about 0.0007 and about 0.06 mM. The fight sensitive agent (such as HEPES) may be present in the medium at a concentration of between about 1 and about 10 mM. The light sensitive agent (such as HEPES) may be present in the medium at a concentration of between about 0.001 and about 0.9 mM. The fight sensitive agent (such as HEPES) may be present in the medium at a concentration of between about 11 and about 20 mM.

A medium may include serum. A medium may not include serum. A medium may be chemically defined.

A medium may have an osmolality between about 280 and about 330 Osm/L. A medium may have an osmolality between about 200 and about 279 Osm/L. A medium may have an osmolality between about 331 and about 400 Osm/L. A medium may have an osmolality between about 280 and about 310 Osm/L. A medium may have an osmolality between about 300 and about 330 Osm/L.

A medium may have an osmolality of about 275 Osm/L. A medium may have an osmolality of about 290 Osm/L. A medium may have an osmolality of about 305 Osm/L. A medium may have an osmolality of about 315 Osm/L. A medium may have an osmolality of about 320 Osm/L. A medium may have an osmolality of about 330 Osm/L. A medium may have an osmolality of about 335 Osm/L A medium may have an osmolality of about 340 Osm/L.

A medium when contacted with an isolated cell may maintain one or more in vivo-like functions of the isolated cell in an ex vivo environment. For example, a medium may maintain an isolated neuron in an vivo-like neurophysiological function by preserving one or more of synaptic function, action potential generation, energetic maintenance, or any combination thereof. An isolated cell cultured in an ex vivo environment may include an environment that is not an in vivo environment. An ex vivo environment may include an environment that does not include one or more incubator conditions (from about 3% to about 8% CO2 content; from about 34 degrees C. to about 40 degrees C.; and from about 75% to about 85% humidity). An ex vivo environment may include an environment lacking one or more incubator conditions.

A medium may include one or more inorganic salts, one or more amino acids, one or more vitamins, one or more other components, or any combination thereof. A medium may include 9 inorganic salts, 18 amino acids, 9 vitamins, or any combination thereof. A medium may include from 8 to 10 inorganic salts, from 16 to 20 amino acids, from 8 to 10 vitamins, or any combination thereof. A medium may include from 6 to 12 inorganic salts, from 14 to 22 amino acids, from 6 to 12 vitamins, or any combination thereof. A medium may include from 4 to 14 inorganic salts, from 12 to 24 amino acids, from 4 to 14 vitamins, or any combination thereof.

A medium may include 1, 2, 3, 4, 5, 6, 7, 8, 9, 10, 11, 12, 13, 14, 15 inorganic salts. A medium may include from 7 to 11 inorganic salts. A medium may include from 8 to 10 inorganic salts. A medium may include 1, 2, 3, 4, 5, 6, 7, 8, 9, 10, 11, 12, 13, 14, 15, 16, 17, 18, 19, 20, 21, 22, 23, 24 or 25 amino acids. A medium may include from 15 to 25 amino acids. A medium may include from 18 to 22 amino acids. A medium may include 1, 2, 3, 4, 5, 6, 7, 8, 9, 10, 11, 12, 13, 14, 15, 16, 17, 18, 19, or 20 vitamins. A medium may include from 5 to 15 vitamins. A medium may include from 7 to 12 vitamins.

A medium may comprise one or more components. The one or more components may include sodium chloride, potassium chloride, calcium chloride, magnesium sulfate, zine sulfate, ferric nitrate, sodium bicarbonate, sodium phosphate dibasic, sodium phosphate monobasic, L-isoleucine, L-threonine, L-leucine, L-valine, L-lysine hydrochloride, L-alanyl-L-glutamine, L-arginine hydrochloride, L-tyrosine disodium salt dehydrate, L-phenylalanine, L-histidine hydrochloride-H20, L-methionine, L-proline, L-cysteine hydrochloride-H20, L-tryptophan, L-asparagine-H20, L-alanine, glycine, L-serine, choline chloride, i-inositol, niacinamide, pyridoxine hydrochloride, thiamine hydrochloride, D-calcium pantothenate, folic acid, vitamin B12, riboflavin, d-glucose, sodium pyruvate, cholesterol, HEPES, or any combination thereof.

A medium may include one or more components, such as one or more buffering agents A buffering agent may increase the life span of an isolated cell as compared to a cell cultured in a medium without the buffering agent. A buffering agent added to a medium may increase the life span of an isolated cell cultured outside of incubator conditions (from about 3% to about 8% CO2 content; from about 34 degrees C. to about 40 degrees C.; and from about 75% to about 85% humidity) as compared to a cell cultured in a medium without the buffering agent also outside of incubator conditions. A buffering agent may increase the life span of the isolated cell by about: 1 day, 1 week, 2 weeks, 3 weeks, 1 month, 2 months, 3 months, 4 months, 5 months, 6 months, 7 months, 8 months, 9 months, 10 months, 11 months, 12 months or more. A buffering agent may increase the life span of the isolated cell by about from 1 week to 1 month. A buffering agent may increase the life span of the isolated cell by about from 1 month to 3 months. A buffering agent may increase the life span of the isolated cell by about from 1 month to 1 year. A buffering agent may increase the life span of the isolated cell by about from 1 month to 6 months.

Outside of incubator conditions may be a deviation from one or more ranges of from about 3% to about 8% CO2 content, from about 34 degrees C. to about 40 degrees C., and from about 75% to about 85% humidity. Outside of incubator conditions may be a deviation outside of from about 3% to about 8% CO2 content, such as about 1% or 10%. Outside of incubator conditions may be a deviation outside of from about 34 degrees C. to about 40 degrees C., such as about 30 degrees C. or about 44 degrees C. Outside of incubator conditions may be a deviation outside of from about 75% to about 85% humidity, such as about 70% or about 90% humidity.

A buffering agent may maintain a pH of the medium, such as a pH from about 7.3 to about 7.5. A buffering agent may maintain a pH of the medium outside of incubator conditions (from about 3% to about 8% CO2 content; from about 34 degrees C. to about 40 degrees C.; and from about 75% to about 85% humidity). A buffering agent may be added to the medium resulting in a final concentration of about: 5 millimolar (mM), 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 30 mM, or 35 mM. A buffering agent may be added to the medium resulting in a final concentration of about 10 mM. A buffering agent may be added to the medium resulting in a final concentration of about 15 mM. A buffering agent may be added to the medium resulting in a final concentration of about 20 mM. A buffering agent may be added to the medium resulting in a final concentration of about 25 mM. A buffering agent may be added to the medium resulting in a final concentration of about 30 mM. A buffering agent may be added to the medium resulting in a final concentration of from about 10 mM to about 30 mM. A buffering agent may be added to the medium resulting in a final concentration of from about 15 mM to about 25 mM A buffering agent may be added to the medium resulting in a final concentration of from about 5 mM to about 25 mM. A buffering agent may be added to the medium resulting in a final concentration of from about 20 mM to about 35 mM.

A buffering agent may include HEPES. A buffering agent may include acetate, N-(2-acetamido)-aminoethanesulfonic acid (ACES), N-(2-acetamido)-iminodiacetic acid (ADA), 2-amino-2-methyl-1-propanol (AMP), N,N-Bis-2-hydroxyethyl)-2-aminoethanesulfonic acid (BES), bicarbonate, bicine, [bis-(2-hydroxyethyl)-imino]-tris-(hydroxymethylmethane) (bis-tris), 1,3-bis[tris(hydroxymethyl)-methylamino]propane (bis-tris-propane), borate, cacodylate, 3-(cyclohexylamino)-propanesulfonic acid (CAPS), 3-(cyclohexylamino)-2-hydroxy-1-propanesulfonic acid (CAPSO), cyclohexylaminoethanesulfnoic acid (CHES), citrate, 3-[N-bis(hydroxyethyl)amino]-2-hydroxypropanesulfonic acid (DIPSO), glycine, glycylglycine, N-(2-hydroxyethyl)-piperazine-N′-ethanesulfonic acid (HEPES), N-(2-hydroxyethyl)-piperazine-N′-3-propanesulfonic acid (HEPPS EPPS), N-(2-hydroxyethyl)-piperazine-N′-2-hydroxypropanesulfonic acid (HEPPSO), imidazole, maleic acid, 2-(N-morpholino)-ethanesulfonic acid (MES), 3-(N-morpholino)-propanesulfonic acid (MOPS), 3-(N-morpholino)-2-hydroxypropanesulfonic acid (MOPSO), phosphate, piperazine-N,N′-bis(2-ethanesulfonic acid (PIPES), piperazine-N,N′-bis(2-hydroxypropanesulfonic acid (POPSO), 3-{[tris(hydroxymethyl)-methyl]-amino}-propanesulfonic acid (TAPS), 3-[N-tris(hydroxymethyl)-methylamino]-2-hydroxypropanesulfonic acid (TAPSO), triethanolamine (TEA), 2-[tris(hydroxymethyl)-methylamino]-ethanesulfonic acid (TES), tricine, tris, or any combination thereof.

A medium may include one or more components, such as one or more antioxidants. An antioxidant may increase the life span of an isolated cell as compared to a cell cultured in a medium without the antioxidant. An antioxidant added to a medium may increase the life span of an isolated cell cultured outside of incubator conditions (from about 3% to about 80,% CO2 content; from about 34 degrees C. to about 40 degrees C.; and from about 75% to about 85% humidity) as compared to a cell cultured in a medium without the antioxidant also outside of incubator conditions. An antioxidant may increase the life span of the isolated cell by about: 1 day, 1 week, 2 weeks, 3 weeks, 1 month, 2 months, 3 months, 4 months, 5 months. 6 months, 7 months, 8 months, 9 months, 10 months, 11 months, 12 months or more. An antioxidant may increase the life span of the isolated cell by about from 1 week to 1 month. An antioxidant may increase the life span of the isolated cell by about from 1 month to 3 months. An antioxidant may increase the life span of the isolated cell by about from 1 month to 1 year. An antioxidant may increase the life span of the isolated cell by about from 1 month to 6 months.

A medium may include one or more antioxidants. The one or more antioxidants may be different antioxidants. The one or more antioxidants may be different types of antioxidants. The medium may include 1, 2, 3, 4, 5, 6, 7, 8, 9, 10 or more antioxidants. The medium may include from 1 to 5 antioxidants. The medium may include from 1 to 3 antioxidants. The medium may include from 1 to 10 antioxidants. The medium may include from 2 to 10 antioxidants. The medium may include any combination of antioxidants provided herewith. The medium may include a vitamin antioxidant, a mineral antioxidant, a protein antioxidant, an amino acid antioxidant, an enzyme antioxidant, a phytonutrient/phytochemical antioxidant, a hormone antioxidant, or other antioxidant.

A medium may include one or more of vitamin A, vitamin C, vitamin D, vitamin E, vitamin K, copper, manganese, iodide, zinc, selenium, magnesium, alpha lipoic acid, glutathione, l-carnosine, glutamate, aspartate, l-carnitine, SOD, DHEA, coenzyme Q10, melatonin, superoxide dismutase, catalase, glutathione peroxidase, lutein, lycopene, zeaxanthin, an ellagic acid, Ginkgo biloba, pycnogenol, resveratrol, glucosinolate, phytoestrogen, allyls sulfide, phenolic acid, water or any combination thereof.

A vitamin antioxidant may be vitamin A, vitamin C, vitamin D, vitamin E, vitamin K, or any combination thereof. A mineral antioxidant may be copper, manganese, iodide, zinc, selenium, magnesium, or any combination thereof. A protein antioxidant may be alpha lipoic acid, glutathione, or any combination thereof. An amino acid antioxidant may be 1-carnosine, glutamate, aspartate, or any combination thereof. An enzyme antioxidant may be 1-carnitine, SOD, DHEA, coenzyme Q10, melatonin, superoxide dismutase, catalase, glutathione peroxidase, citric acid, oxalic acid, phytic acid, or any combination thereof. A phytonutrient/phytochemical antioxidant may be a carotenoid (such as lutein, lycopene, zeaxanthin, or any combination thereof), an ellagic acid, a flavonoid (such as Ginkgo biloba, pycnogenol, or any combination thereof), resveratrol, glucosinolate, phytoestrogen, allyls sulfide, phenolic acid, or any combination thereof. A hormone antioxidant may be melatonin, DHEA, or a combination thereof. Water may be an antioxidant. An antioxidant may be citric acid, oxalic acid, phytic acid, or any combination thereof. An antioxidant may be water-soluble, fat-soluble, or a combination thereof.

An antioxidant may be present in the medium at a concentration of between about 0.000001 and about 0.00001 mM. An antioxidant may be present in the medium at a concentration of between about 0.00001 and about 0.0001 mM. An antioxidant may be present in the medium at a concentration of between about 0.0001 and about 0.001 mM. An antioxidant may be present in the medium at a concentration of between about 0.001 and about 0.01 mM. An antioxidant may be present in the medium at a concentration of between about 0.01 and about 0.1 mM. An antioxidant may be present in the medium at a concentration of between about 0.1 and about 1 mM. An antioxidant may be present in the medium at a concentration of between about 1 and about 10 mM. An antioxidant may be present in the medium at a concentration of between about 0.000001 and about 0.001 mM. An antioxidant may be present in the medium at a concentration of between about 0.00001 and about 0.01 mM. An antioxidant may be present in the medium at a concentration of between about 0.001 and about 0.1 mM.

### Receptors and Compound Detection

A cell receptor may detect a presence of a compound. A cell receptor may detect a presence of a compound, such as when a compound binds to the cell receptor. A binding event between a cell receptor and a compound may result in a signal, such as a light signal or electrical signal or chemical signal.

A cell receptor that may detect a presence of a compound may be a wild type receptor. A cell receptor that may detect a presence of a compound may be a modified receptor, such as a genetically modified receptor. A cell receptor that may detect a present of a compound may be an odorant receptor.

A cell, such as a neuron, may be modified to include one or more wild type receptors, one or more modified receptors, or any combination thereof. A cell may be modified to include a receptor that detects a single compound. A cell may be modified to include a receptor that detects more than one compound. A cell may be modified to include more than one type of receptor, such as a first type of receptor that detects a first compound and a second type of receptor that detects a second compound. A cell may be modified to include 1, 2, 3, 4, 5, 6, 7, 8, 9 or 10 different types of receptors.

A binding event between a cell receptor and a compound may occur in a liquid medium. A binding event may occur in a semi-solid medium. A binding event may occur in a viscous medium. A binding event may occur in an aqueous medium. A binding event may occur in a hydrogel.

A cell receptor may detect a presence of a compound at a concentration of about 10 nM or less A cell receptor may detect a presence of a compound at a concentration of about 25 nM or less. A cell receptor may detect a presence of a compound at a concentration of about 50 nM or less A cell receptor may detect a presence of a compound at a concentration of about 75 nM or less A cell receptor may detect a presence of a compound at a concentration of about 100 nM or less. A cell receptor may detect a presence of a compound at a concentration of about 250 nM or less. A cell receptor may detect a presence of a compound at a concentration of about 500 nM or less. A cell receptor may detect a presence of a compound at a concentration of about 750 nM or less. A cell receptor may detect a presence of a compound at a concentration of about 10 uM or less. A cell receptor may detect a presence of a compound at a concentration of about 25 uM or less A cell receptor may detect a presence of a compound at a concentration of about 50 uM or less. A cell receptor may detect a presence of a compound at a concentration of about 75 uM or less. A cell receptor may detect a presence of a compound at a concentration of about 100 uM or less. A cell receptor may detect a presence of a compound at a concentration of about 250 uM or less.

A cell may include one or more of mOR185-1, mOR189-1, mOR9-1, mOR162-2, mOR202-37, mOR204-3, mOR204-8, mOR204-11, mOR188-1, mOR256-3, any genetic variation thereof, any human analog thereof, any functionally active fragment thereof or any combination thereof.

A cell may include one or more of mOR156-5, mOR159-3, mOR160-4, mOR160-5, mOR161-5, mOR161-6, mOR162-2, mOR164-2, mOR202-37, mOR204-3, mOR204-8, mOR204-11, any genetic variation thereof any human analog thereof any functionally active fragment thereof, or any combination thereof.

A cell may include one or more of mOR165-1, mOR178-1, mOR179-1, mOR183-1, mOR185-1, mOR162-1, mOR189-1, any genetic variation thereof, any human analog thereof any functionally active fragment thereof or any combination thereof.

A cell may include one or more of receptors listed in Table 2b, Table 3, Table 4, any genetic variation thereof any human analog thereof any functionally active fragment thereof: or any combination thereof.

A compound that may be detected by a cell receptor may include one or more volatile organic compounds. A compound may include DNT (CAS #121-14-2), RDX (CAS #121-82-4), TNT (CAS #118-96-7), vanillic acid (CAS #121-34-6), or any combination thereof.

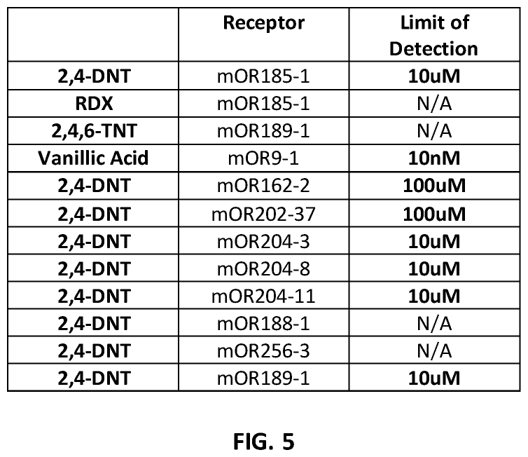

FIG. 5 shows a table of compounds (left column) and receptors that detect such compounds (middle column) and the concentration limit of detection for each compound receptor pair (right column). For example, 2,4-DNT (CAS #121-14-2) may be detected by receptor mOR185-1 at a concentration limit of detection of about 10 uM. A single receptor may detect more than one compound. Multiple receptors may detect the same compound at different concentration limits of detection.

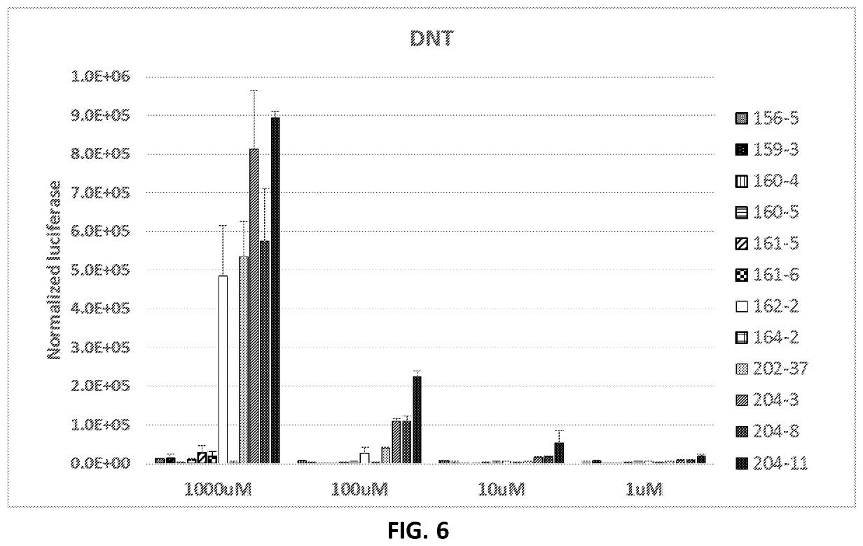

FIG. 6 shows a range of DNT (CAS #121-14-2) concentrations (1 microMolar (uM) to 1000 (uM)) and respective odorant receptor detection as reported by measured levels of luminescence. The y axis of the bar graph reports measured normalized luciferase activity. The x axis of the bar graph reports different concentration limits of detection. The right hand side of the graph reports different receptors tested. Some receptors show a lower limit of detection of DNT than other receptors.

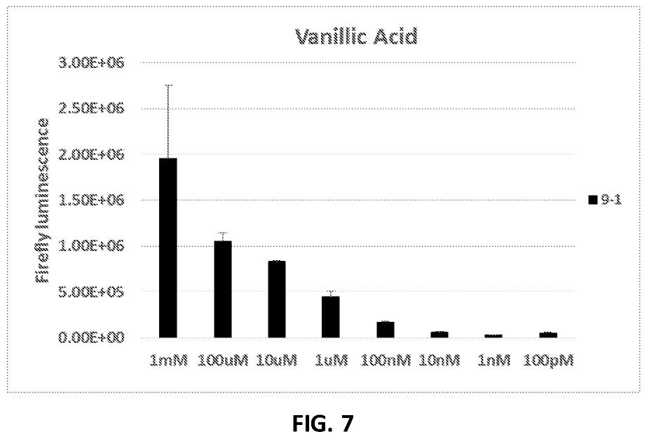

FIG. 7 shows a range of vanillic acid (CAS #121-34-6) concentrations (100 picoMolar (pM) to 1 milliMolar (mM)) and respective odorant receptor detection as reported by measured levels of luminescence. The y axis of the bar graph reports measured luminescence. The x axis of the bar graph reports different concentration limits of detection. The right hand side of the graph reports the receptor mOR9-1 tested. A lower limit of detection for vanillic acid by receptor mOR9-1 may be about 10 nM.

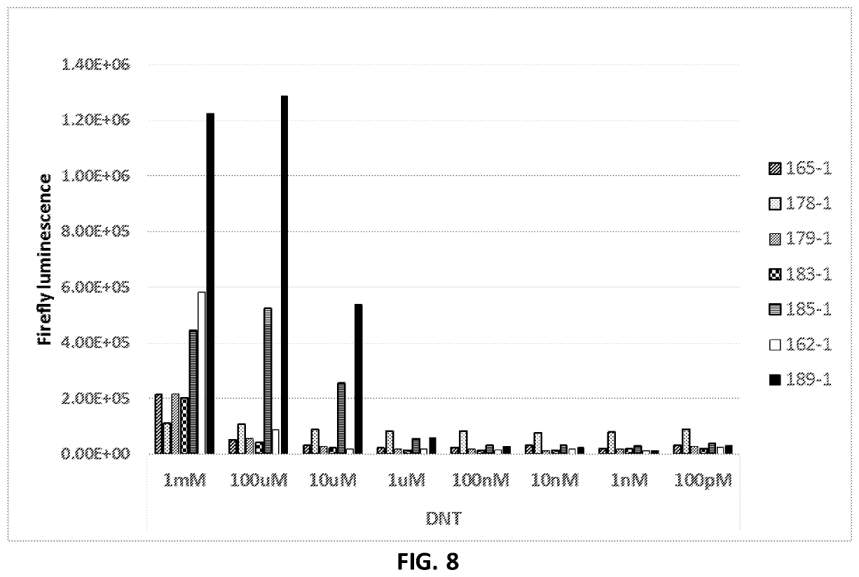

FIG. 8 shows a range of DNT (CAS #121-14-2) concentrations (100 picoMolar (pM) to 1 millimolar (mM)) and respective odorant receptor detection as reported by measured levels of luminescence. The y axis of the bar graph reports measured luminescence. The x axis of the bar graph reports different concentration limits of detection. The right hand side of the graph reports different receptors tested. Some receptors show a lower limit of detection of DNT than other receptors.

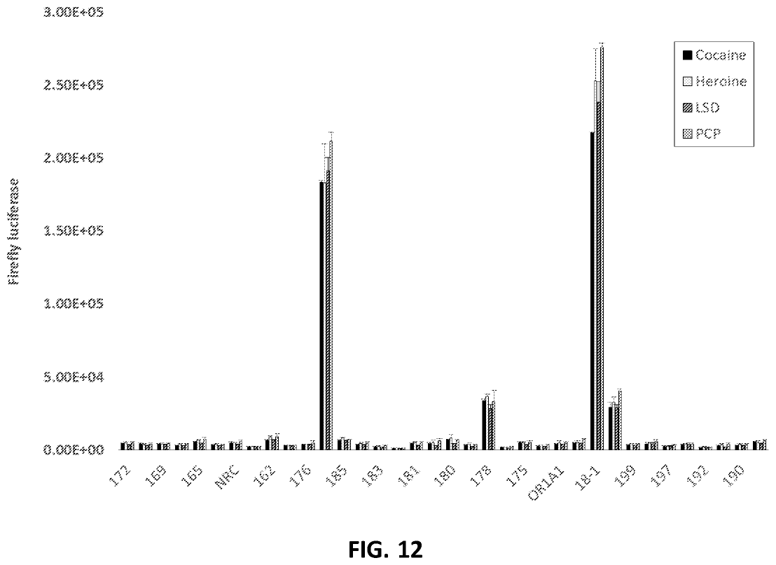

FIG. 12 shows odorant receptor detection of four different compounds: cocaine, heroine, LSD, and PCP as measured by levels of luminescence for a panel of different odorant receptor types. A subset of odorant receptors tested (such as mOR18-2, mOR178-1, mOR18-1 and OR2W1) responded to all four compounds tested as indicated by the measured levels of luminescence. Broadly tuned receptor, such as those receptors that detect more than one type of compound, may help detect a subset of compounds without being able to identify the chemical nature of the compound being detected.

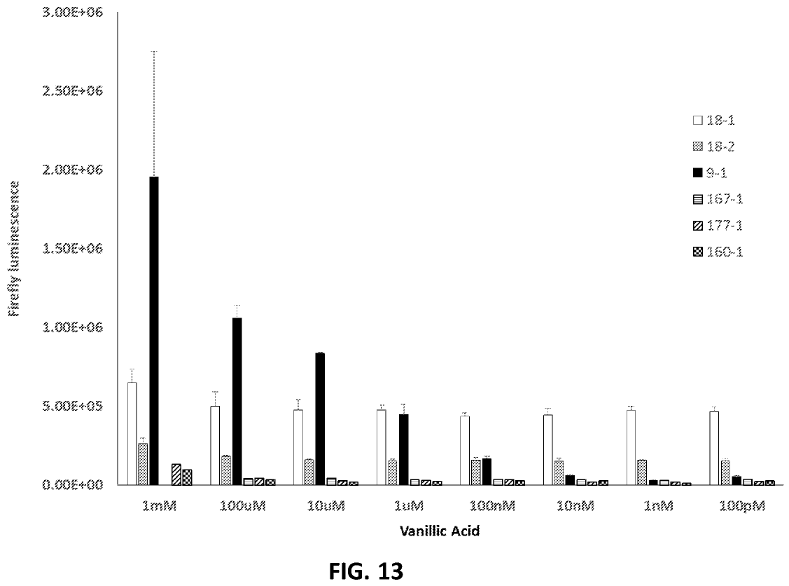

FIG. 13 shows odorant receptor detection of vanillic acid as measured by levels of luminescence at different concentrations for six different types of odorant receptors. Odorant receptor mOR9-1 demonstrates a decreasing level of luminescence as the concentration of vanillic acid is decreased. Odorant receptors mOR18-1 and mOR18-2 demonstrate similar levels of luminescence as the concentration of vanillic acid is decreased. These receptors may respond to other environmental stimuli. Odorant receptors mOR177-1 and mOR160-1 show low background signal in the presence of different concentrations of vanillic acid.

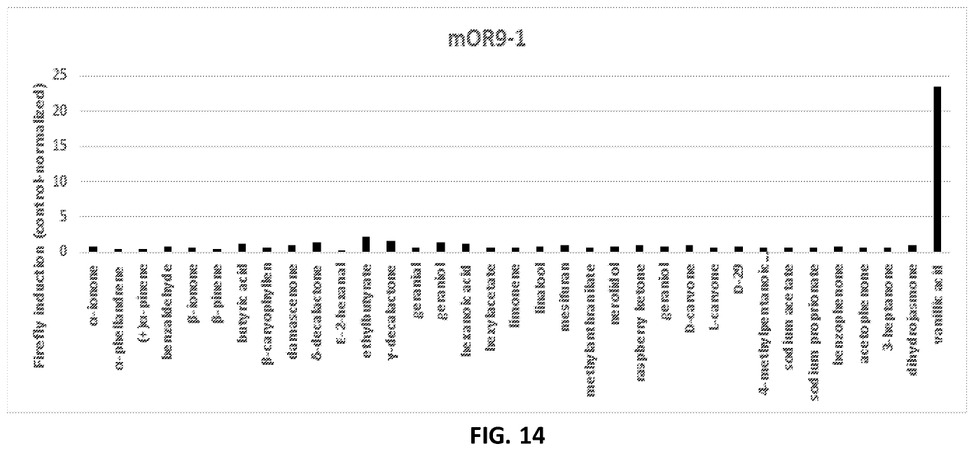

FIG. 14 shows odorant receptor detection as measured by levels of luminescence for a panel of different types of compounds for mouse odorant receptor (mOR9-1). Based on the range of different compounds tested, mOR9-1 may be more narrowly tuned to one (vanillic acid) compound or a smaller subset of ligands. A narrowly tuned receptor, may help detect a specific compound or a specific smaller subset of compounds.

### Signal Detection

Devices as described herein may include one or more isolated cells (such as a neuron). The isolated cell may be utilized as a sensing front-end to detect specific volatile organic compounds from or within an environment. An isolated cell may sense a presence of a compound in real-time in an environment, or from an isolated sample (air sample, liquid sample, solid sample) that was previously taken from an environment to be tested.

One or more isolated cells within the device may alert the detection system that a binding event (between a cell receptor and a compound) has occurred by creating one or more of a biological signal, a light signal, a chemical signal, an electrical signal, a vibration signal, a mechanical signal, or any combination thereof. In the case of a neuron, the signal may comprise firing an electrical impulse, such as an action potential or a portion of an action potential A signal emitted from an isolated cell of the device may be detected by another component of the device, such as a sensor. In the case of an electrical signal, such as an action potential, one or more electrodes (such as a gold electrode) may receive this electrical signal. Electrodes of an device may sense one or more ionic fluxes including one or more of Na+, Ca2+, K+ fluxes, or combination thereof. These ionic fluxes may pass in and out of the membrane of the isolated cell to generate one or more electrical signals, and the resulting electron flow may be translated to one or more electrodes, which may then be translated by an analogue to digital converter to a waveform which may then be classified by downstream algorithms as either an electrical signal (i.e., action potential or portion thereof) or noise.

The difference between a noise signal and an electrical signal (i.e., action potentials or portion thereof) may be termed the signal-to-noise ratio (SNR). The SNR may determine a confidence level of a detection event. The SNR may define a level of signal power with respect to a level of noise power, where noise may be a result of a physical turbulence or electromagnetic noise emanating from one or more surrounding electronics. The quality of a cellular recording (such as recording action potentials of a neuron in response to the presence of a compound) may be determined by an amplitude of a voltage of a recorded spike. This amplitude may be increased by improving a contact interface between an isolated cell (such as a neuron) and a surface of an electrode. In some cases, increasing a contact interface may include reducing a physical distance between the isolated cell and a surface of the electrode, increasing a contact surface area between the isolated cell and the electrode, or a combination thereof. Both approaches of improving contact interface may be optimized by augmenting electrode geometry to encourage the isolated cell (i.e., neuron) to engulf the electrode, such as a geometry that may advantageously trigger one or more endocytotic pathways in the isolated cell. By triggering one or more endocytotic pathways in an isolated cell, the isolated cell may begin to consume at least a portion of the electrode via partial phagocytosis, which may increase the cell membrane surface area participating in ion exchange that may be in close proximity to the electrode for an electrical current to be generated in a portion of the electrode and amplified by an amplifier and digital to analogue converter.

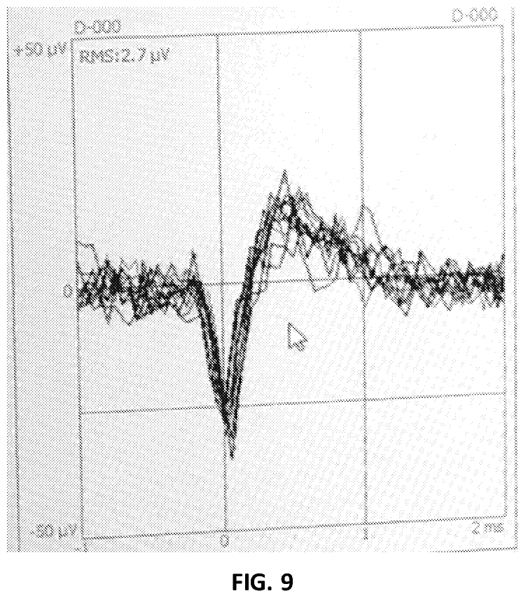

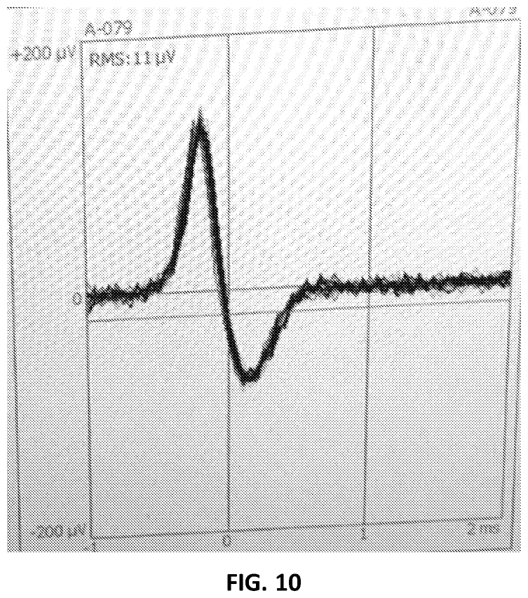

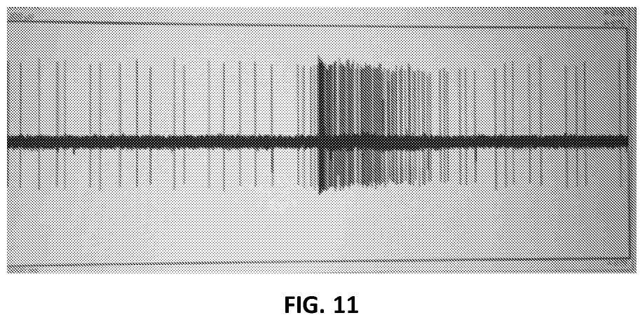

FIG. 9 shows an amplitude gain obtained employing a planar 2D electrode geometry. The image is generated by Intan recording software. The image demonstrates an average of about 50/3 uV peak-to-peak signal quality based on a distance between the maximum and minimum peaks present in the image. FIG. 10 shows a superior amplitude gain obtained employing a 3D electrode geometry showing far superior amplitude gain as compared to the planar 2D electrode geometry. This image demonstrates an average of about 200/3 uV peak-to-peak signal quality based on the distance between the maximum and minimum peaks present in the image. In addition, the signal-to-noise (SNR) ratio is significantly improved using the 3D electrode geometry in FIG. 10 as compared to the 2D electrode geometry in FIG. 9. A 3D electrode geometry may result in a four fold signal gain over 2D electrode geometry. A 3D electrode geometry may result in a 5, 6, 7, 8, 9 or 10 fold signal gain over 2D electrode geometry. FIG. 11 shows a spike train obtained using a 3D electrode geometry.

### Devices for Detection

The devices, systems, and methods as described herein may be employed to confirmation a presence or an absence of one or more compounds in a sample. In some embodiments, the confirmation may be a likelihood of a presence or a likelihood of an absence of one or more compounds in a sample. A sample may be a blood sample, bodily fluid, tissue sample, or any combination thereof. In such embodiments, a presence or absence of an illegal compound in a subject's body may be confirmed. A sample may be a soil sample, a water sample, or a gas sample. In such embodiments, a presence or absence of a chemical weapon, a toxin, a carcinogen in a soil, in a waterway, in an air supply, in a geographical region, in a residential setting in a commercial setting may be confirmed.

In some embodiments, the system may comprise an array of cells. These cells may be from any suitable origin. For example, they may be simulated, synthetic or natural. The array may be intended to provide the cells with life support, capable of maintaining a suitable environment for these cells, including temperature control and delivering nutrients and other materials to the cells. As such, the array may be combined with other systems for the transportation of living cells, without interruption to normal physiological functions. The preferred embodiments of the disclosure provide electrodes for interaction with the cells, in order to monitor signals, such as electrical signals. Preferred embodiments of the disclosure also allow delivery of compounds to the cells and monitoring the cell response. Preferred embodiments of the disclosure may also support imaging modalities to monitor the cell response or function.

In general, the array may provide a structure (which can be considered to be a substrate (in x, y, z coordinates)) in which the cells may be housed, the same structure providing an arrangement for perfusion of the cells with nutrients, growth media, growth factors, compounds, etc.

The array may be intended to form part of a base unit, providing a gas delivery system which drives the perfusion of cell culture media through the array. Additional elements of the array may include heating elements, for maintaining life support in the array. Various sensors may also be included to monitor the temperature, pH, gas species, particle analysis, etc., in closed loop.

In addition to the foregoing, other sensors may be provided, e.g. adapted to sense the presence of specific proteins or ionic molecules. Such sensors and detectors can interface with the array in a secondary manner to provide analytic read outs for genomics, proteomics, western blot assays and other lab-on-chip devices.

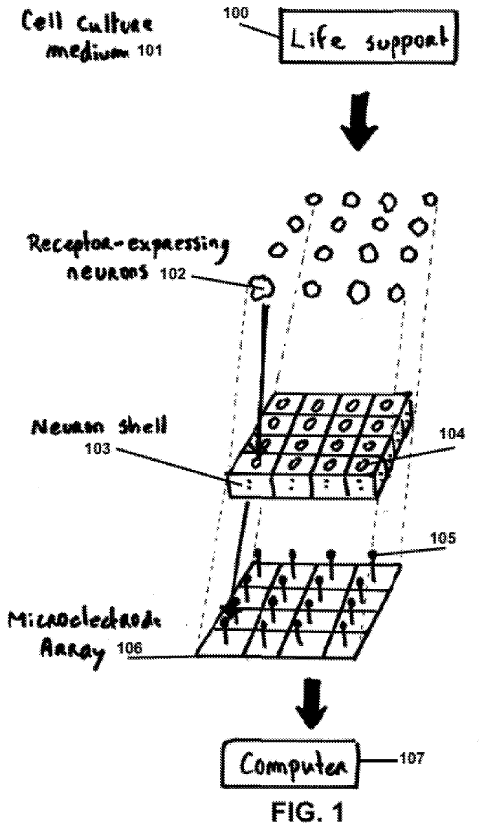

In FIG. 1, shows a life support system 100 that may be operatively connected to the array of cells. The life support system may provide a controlled environment of a liquid volume, such as a cell culture medium 101, under a controlled pH, a controlled temperature, a controlled osmolality, or combinations thereof. Compounds of interest may be dissolved into the liquid volume. One or more compounds added to the liquid volume may interact or bind to one or more receptors of receptor-expressing neurons 102 of the array which may trigger a signal, such as an electrical signal. The neurons may reside in a neuron shell 103. The neurons may be individually placed through an opening at the top of each neuron shell, represented by a small circle 104 in FIG. 1. Each neuron shell may be positioned about a subset of microelectrodes represented by a single electrode 105 in each square of the grid 106. One or more electrical signals may be collected from the neurons excited by one or more compounds. The one or more electrical signals measured by the electrodes may be received by a controller, such as a computer 107 as represented by the black arrow at the bottom.

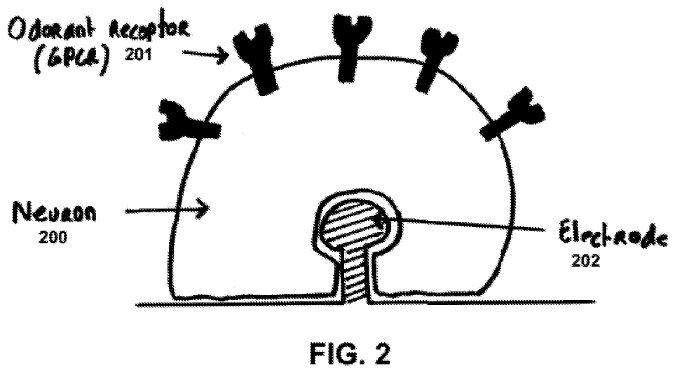

In FIG. 2, a neuron 200 expressing more than one odorant-receptor 201 may be placed atop a subset of electrodes (such as gold microelectrodes) represented in FIG. 2 by one electrode 202. Compounds of interest may bind to the odorant-receptor 201 leading to a cascade of events in the cell cytoplasm of the neuron 200 which may lead to an electrical signal, such as a membrane depolarization and possibly an action potential or a train of action potentials. The electrical signal may be measured or recorded by the electrode 202.

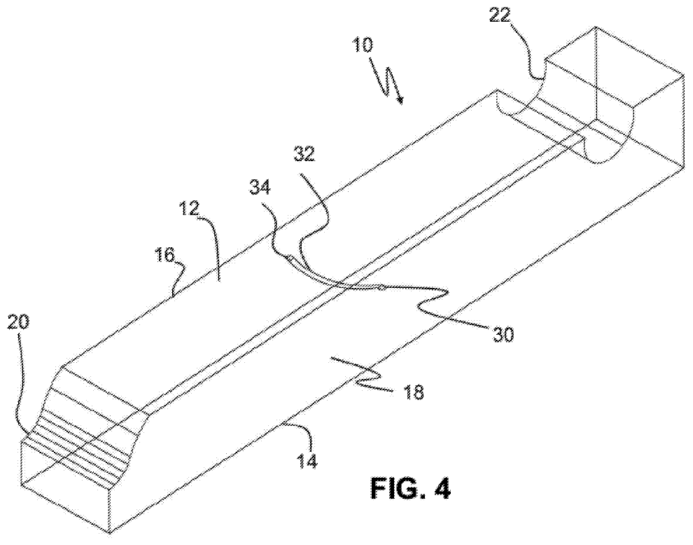

In FIG. 4, an army may be generally indicated by reference number 10. This may have a generally elongate prismatic shape with an upper region bounded by upper surface 12 and a lower region bounded by lower surface 14. Upright side surfaces 16, 18 may also be provided. Front region 20 may be provided with a profile surface, for integration of the array into the system. Additionally, a cut-out 22 may be formed towards the rear part of the upper region of the array. The front and rear shapes of the array may provide purchase for a hooking system to be able to pick up the array and place it in a base unit (not shown).

In FIG. 4, chamber 30 is shown, located in the lower region of the module. Chamber 30 may house one or more cells (not shown). Chamber 30 may have a respective cell introduction passage 32, leading from cell introduction port 34 at upper surface 12 of the module. As can be seen in FIG. 1, the cell introduction passage may slope and curve generally downwardly from the upper region to the chamber in the lower region. This feature may permit one or more cells to be introduced into chamber 30 via port 34 and passage 32, and may provide certainty as to the number and type of cells contained in chamber 30.

In some embodiments, the system may comprise an army of cells, such as neurons, that may be modified, such as genetically modified, to express cell-surface receptors. The cell-surface receptors may detect one or more compounds such as a volatile organic compound or a water-soluble odorant compound. A cell of the system may express a single type of compound-sensing receptors or may express multiple types of compound-sensing receptors that may detect a set or a mixture of compounds. The array of cells may comprise one or more cells expressing one or more of these compound-sensing receptors. A cell-surface receptor that may sense a compound may do so via a series of signaling proteins that internally amplify a signal, such as an electrical signal, and may trigger an action potential by the cell, such as a neuron. Each individual cell may be operatively connected to one or more electrodes of the system, such as the microelectrode array (MEA). Following or during a binding event between a compound and a cell-surface receptor, the operative connection between a cell and an electrode may permit detection of one or more cell-based electrical signals, such as a cell membrane depolarization, an action potential, or an electrical signal that is subthreshold of an action potential. An electrical signal response may be transferred to an electrode and to a controller, such as a computer input device. In some embodiments, the array of cells differentially detects an array of compounds, which collectively can yield a compound fingerprint of detection.

A compound (such as an odorant compound) binding to cell-surface receptor (such as an odorant receptor) in a cell (such as a neuron) expressing one or more of that type of cell-surface receptor may active a signaling pathway within the cell. In some embodiments, wherein the cell is a neuron, a binding event may trigger a membrane depolarization of the neuron and in some cases also trigger an action potential, both of which are electrical signals that can be detected by an electrode that may be operatively connected to the cell. In some embodiments, a complete action potential may not be triggered. In such cases, an electrode may still detect an electrical signal from the cell, such as detection of a sub-threshold level signal that may be distinguishable from noise. An excited neuron may generate one or more action potentials to a given binding effect, the one or more action potentials of which may be grouped, stored, or analyzed as a train of action potentials by a controller. A distribution pattern of action potentials within a train of action potentials may also contain electrical signal information which may be collected by the system.

In some embodiments, an array of cells may comprise a plurality of cells, wherein each cell of the plurality expresses a unique cell-surface receptor. In some cases, a compound, such as an odorant, may bind differentially across the plurality of cells such that each cell may be have a different electrical signal level between a baseline line, such as no action potential (i.e. zero), and a full action potential. In some embodiments, different cells may generate a train of action potentials that may have different or unique action potential distributions within the train, depending on the cell-surface receptor that may be expressed on the cell surface.

Delivery of a single compound (such as an odorant) or a set of compounds with known characteristics to an array may provide a series of relative signals across the array that can be obtained and analyzed by the devices, systems, and methods as described herein. Values associated with the relative signals may be imported or contained within a matrix containing different levels (such as a magnitude) and profiles (such as a temporal profile) of electrical signals for each cell, based on sub-threshold signals, full-threshold signals, membrane depolarizations, action potentials, or any combination thereof.

The system may comprise an array of cells, such as array of n×m cells. Signals may be encoded and represented in a matrix where each element may represent a real value aᵢⱼ where a may be the sub-threshold value or a full on/off action potential, and i and j may represent a position on the array of the device.
a₀₀ a₀₁ a₀₂ a₀₃ . . . a₀ₙ
a₁₀ a₁₁ a₁₂ a₁₃ . . . a₁ₙ
a₂₀ a₂₁ a₂₂ a₂₃ . . . a₂ₙ
a₃₀ a₃₁ a₃₂ a₃₃ . . . a₃ₙ
. . .
aₘ₀ aₘ₁ aₘ₂ aₘ₃ . . . aₘₙ

A single compound may bind to different cell-surface receptors with different binding affinities. A single compound may have a strong binding affinity to one type of cell-surface receptor and a weak binding affinity for a second type of cell-surface receptor. Different binding affinities, such as a strong binding affinity and a weak affinity, may result because a binding to a G protein coupled receptor (GPCR) is a 3-dimensional binding event. In some embodiments, binding sites of cell-surface receptors may not be sensitive enough to recognize particular moieties or chemical substituents (e g, OH, CH3, etc.) that comprise a given compound of interest. Instead, it may be that a combination of features of the compound may be sufficient to provide a ligand “shape” or a conformation that permit a binding event to occur between the compound and the cell-surface receptor inside the GPCR binding pocket. This, different parts of a compound may bind to different cell-surface receptors differently and may trigger different signals in different cells on the array. In some embodiments, a cell that binds to a compound with a strong binding affinity may provide a signal in response to that binding event that is different that an electrical signal produces by a cell that may have a weak binding affinity for the same compound. In some embodiments, cell-surface receptor binding sites may be sensitive enough to recognize particular moieties or chemical substituents (e.g., OR CH3, etc.) that comprise a compound of interest.

A compound may bind different receptors with different binding affinities. For example, a narrowly tuned receptor (such as a modified receptor that may be modified to bind to a specific compound with a strong binding affinity) may only bind to a limited subset of compounds and a binding event to a compound to which the receptor is not narrowly tuned may not occur or may be less likely to occur. Broadly tuned receptors (such as a modified receptor that may be modified to bind to a wider range of compounds with a strong binding affinity) may bind to a larger number of different compounds with different binding affinities as compared to the narrowly tuned receptor. Broadly tuned receptors may be more likely to be useful in a chemical fingerprint determination. Broadly tuned receptors may be more likely to provide a confirmation of a presence of a compound in a sample with a high probability or degree of confidence, such as 70%, 75%, 80%, 85%, 90%, 95%, 96%, 97%, 98%, 99% or more.

A given compound may have a relatively fixed set of values in a matrix, with a range of variation across all values (such as non-zero values). For example, a first exposure of the given compound to an array of cells, may yield a first set of values in a matrix. A second exposure of the given compound to the same array of cells, may yield a second set of values in the matrix. The first set of values and the second set of values may be relatively the same set of values. The first set of values and the second set of values may have a range of variation across all values, wherein the variation may be not more than 1%, 2%, 3%, 4%, 5%, 6%, 7%, 8%, 9%, 10%, 15%. This relatively fixed set of values of the matrix may be used as a fingerprint or a unique pattern that may be assigned to that particular compound (and may be stored in a database of the system) given the distribution of the cell-surface receptor expressing cells.

A set of compounds (related or unrelated to one another) may have a particular fingerprint when mapped against a particular set of cell-surface receptors expressed by cells of an array. This fingerprint for a given set of compounds may represent an overlapping set of binding events of individual compounds. That is, individual compounds in the set of compounds may bind to more than one cell-surface receptor in different ways. In such a case, a given set of compounds may be additive across the array and the signals from some compounds in the given set may mask or render undetectable the individual signals from other compounds in the set. Each set or combination of compounds may have a unique fingerprint across the array. A pattern of signals generated by a given compound within a mixture of compounds on an array may act as a compound fingerprint which can be recognized within a mixture of compounds thereby allowing a system as described herein to determine individual compounds in a set or mixture of compounds.

In some embodiments, the systems described herein provide an array of cells, such as neurons, having receptors which bind to an odorant or a compound, such as a volatile organic compound, and upon binding, the cells send detection information, such as signals (such as electrical signals), to a controller.

### Computer Systems

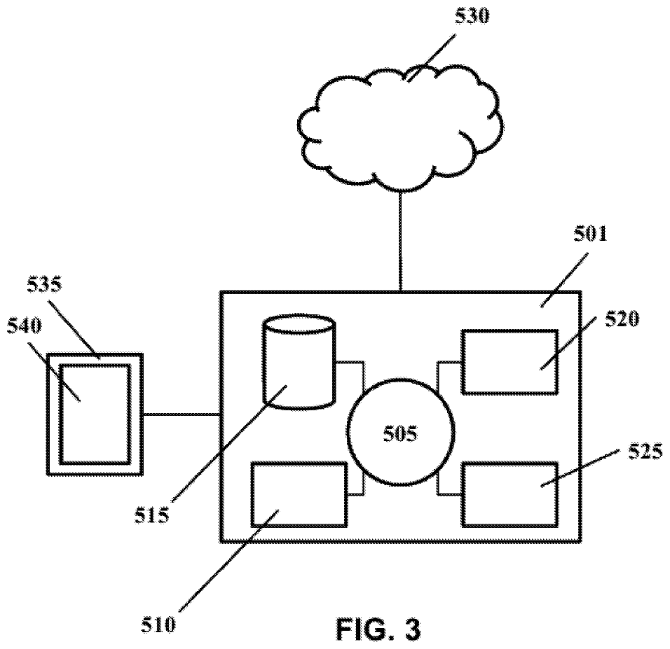

The present disclosure provides computer control systems that are programmed to implement methods of the disclosure. FIG. 3 shows a computer system 501 that may be programmed or otherwise configured to direct electrodes to measure one or more electrical signals, to receive one or more electrical signals from one or more electrodes, to generate a pattern of electrical signals, to store patterns of electrical signals or electrical signals in a database, to compare a pattern of electrical signals to a pattern stored in a database, or any combination thereof. The computer system 501 can regulate various aspects of data collection, data analysis, and data storage, of the present disclosure, such as, for example, directing electrical signal measurements, comparing of patterns based of electrical signals measured, generating patterns based on electrical signal data, any combinations thereof and others. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g. random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or mom other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., portable PC, tablet PC, Smart phones). Examples of remote computer systems include personal computers (e.g., portable PC), slate or tablet PC's (e.g. Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system Carrier-wave transmission media may take the form of electric or electromagnetic signals, or acoustic or fight waves such as those generated during radio frequency (RF) and infrared (IR) data communications. Common forms of computer-readable media therefore include for example, a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, a confirmation of a presence or a likelihood of a presence of a compound, such as a volatile compound. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, generate a pattern based on electrical signals received from one or more electrodes, such as a matrix of electrical signals, compare a pattern generated by the control system to one or more patterns stored in a database of the system, make a confirmation of a presence or a likelihood of a presence of a compound in sample, or any combination thereof and others.

### SPECIFIC EMBODIMENTS

A number of devices, systems, arrays, methods are disclosed herein. Specific exemplary embodiments of these devices, systems, arrays, methods are disclosed below.

Embodiment 1. A device comprising: (a) a spatially addressable array, the spatially addressable array comprising: a plurality of chambers, wherein each chamber of the plurality of chambers comprises: (i) a cell modified to express a unique odorant receptor profile; and (ii) an electrical component configured to measure an electrical signal in the cell, (b) a controller configured to (i) receive the measured electrical signals from the plurality of chambers and (ii) determine a presence or an absence of one or more compounds based on the measured electrical signals.

Embodiment 2. The device of embodiment 1, wherein the presence or the absence of the one or more compounds comprises measuring an amount of the one or more compounds.

Embodiment 3. The device of embodiment 1 or 2, wherein the cell comprises a genetic modification.

Embodiment 4. The device of any one of embodiments 1-3, wherein the cell is a neuron.

Embodiment 5. The device of any one of embodiments 1-4, wherein the cell is a human cell.

Embodiment 6. The device of any one of embodiments 1-5, wherein the spatially addressable array comprises greater than three unique odorant receptor profiles.

Embodiment 7. The device of any one of embodiments 1-6, wherein the device is configured to detect at least two different types of compounds.

Embodiment 8. The device of any one of embodiments 1-7, wherein the cell detects more than one type of compound.

Embodiment 9. The device of any one of embodiments 1-8, wherein the cell is modified to express at least two unique odorant receptor profiles.

Embodiment 10. The device of any one of embodiments 1-9, wherein the unique odorant receptor profile comprises one or more of the following receptors: OR1A1, mOR106-1, OR51E1, OR10J5, OR51E2, mOR9-1, mOR18-1, mOR272-1, mOR31-1, mOR136-1, mOR185-1, mOR189-1, mOR9-1, mOR162-2, mOR202-37, mOR204-3, mOR204-8, mOR204-11, mOR188-1, mOR256-3, mOR156-5, mOR159-3, mOR160-4, mOR160-5, mOR161-5, mOR161-6, mOR162-2, mOR164-2, mOR202-37, mOR204-3, mOR204-8, mOR204-11, mOR165-1, mOR178-1, mOR179-1, mOR183-1, mOR185-1, mOR162-1, mOR189-1, any genetic variation thereof any functionally active fragment thereof, or any combination thereof.

Embodiment 11. The device of any one of embodiments 1-9, wherein the unique odorant receptor profile comprises one or more of the following receptors: OR1A1, mOR106-1, OR51E1, OR10J5, OR51E2, mOR9-1, mOR18-1, mOR272-1, mOR31-1, mOR136-1, any genetic variation thereof, any functionally active fragment thereof or any combination thereof.

Embodiment 12. The device of any one of embodiments 1-9, wherein the unique odorant receptor profile comprises one or more of the following receptors: mOR185-1, mOR189-1, mOR9-1, mOR162-2, mOR202-37, mOR204-3, mOR204-8, mOR204-11, mOR188-1, mOR256-3, any genetic variation thereof any human analog thereof any functionally active fragment thereof or any combination thereof.

Embodiment 13. The device of any one of embodiments 1-9, wherein the unique odorant receptor profile comprises one or more of the following receptors: mOR156-5, mOR159-3, mOR160-4, mOR160-5, mOR161-5, mOR161-6, mOR162-2, mOR164-2, mOR202-37, mOR204-3, mOR204-8, mOR204-11, any genetic variation thereof any human analog thereof, any functionally active fragment thereof, or any combination thereof.

Embodiment 14. The device of any one of embodiments 1-9, wherein the unique odorant receptor profile comprises one or more of the following receptors: mOR165-1, mOR178-1, mOR179-1, mOR183-1, mOR185-1, mOR162-1, mOR189-1, any genetic variation thereof: any human analog thereof any functionally active fragment thereof or any combination thereof.

Embodiment 15. The device of any one of embodiments 1-9, wherein the unique odorant receptor profile comprises one or more receptors from Table 2b, Table 3, Table 4, any genetic variation thereof any human analog thereof any functionally active fragment thereof or any combination thereof.

Embodiment 16. The device of any one of embodiments 1-15, wherein the unique odorant receptor profile comprises a modification.

Embodiment 17. The device of embodiment 16, wherein the modification comprises one or more of a genetic modification, a methylation modification, a sulfentation modification, a sulfentation modification, a acylation modification, an alkylation modification, a butyrylation modification, a glycosylation modification, a malonylation modification, a hydroxylation modification, an iodination modification, a propanoylation modification, or any combination thereof.

Embodiment 18. The device of embodiment 16, wherein the modification comprises an oxidation modification or reduction modification.

Embodiment 19. The device of embodiment 16, wherein the modification comprises a carbohydrate addition, a carbohydrate deletion, or a combination thereof.

Embodiment 20. The device of any one of embodiments 1-19, wherein the controller determines a result comprising the presence or the absence of the compound.

Embodiment 21. The device of embodiment 19 or embodiment 20, wherein the controller communicates the result via a communication medium.

Embodiment 22. The device of any one of embodiments 20-21, wherein the result comprises a probability of the presence or the absence of the compound.

Embodiment 23. The device of embodiment 22, wherein the probability is from about 80% to about 99%.

Embodiment 24. The device of embodiment 22, wherein the probability is at least about 85%.

Embodiment 25. The device of any one of embodiments 1-24, wherein the device detects the presence or the absence of the compound at a concentration detection limit of about 10 millimolar (mM) or less.

Embodiment 26. The device of embodiment 25, wherein the concentration detection limit is about 10 micromolar (uM) or less.

Embodiment 27. The device of any one of embodiments 1-26, wherein the controller stores one or more signaling patterns associated with one or more reference compounds in a database.

Embodiment 28. The device of any one of embodiments 1-27, wherein the measured electrical signals comprise an action potential, a cell membrane depolarization, an excited signal level that is below a threshold for an action potential, or any combination thereof.

Embodiment 29. The device of any one of embodiments 1-28, wherein the measured electrical signals comprise a compound-specific pattern.

Embodiment 30. The device of any one of embodiments 1-30, wherein the measured electrical signals comprise a binary pattern.

Embodiment 31. The device of any one of embodiments 1-28, wherein the measured electrical signals comprise a magnitude of an electrical signal, a temporal pattern of an electrical signal, or a combination thereof.

Embodiment 32. The device of any one of embodiments 1-31, wherein the electrical component comprises an electrode.

Embodiment 33. The device of embodiment 32, wherein the electrode is a three-dimensional electrode.

Embodiment 34. The device of embodiment 33, wherein the three-dimensional electrode comprises a support portion and a head portion.

Embodiment 35. The device of embodiment 34, wherein the head portion comprises a protrusion, a recess, or a combination thereof.

Embodiment 36. The device of embodiment 35, wherein the protrusion comprises a hemispherical protrusion.

Embodiment 37. The device of embodiment 35, wherein the recess comprises a hemispherical recess.

Embodiment 38. The device of any one of embodiments 33-37, wherein the three-dimensional electrode comprises at least 100 protrusions, at least 100 recesses, or any combination thereof.

Embodiment 39. The device of any one of embodiments 33-38, wherein the three-dimensional electrode comprises a surface roughness from about 10 nanometers (nm) to about 100 nm.

Embodiment 40. The device of any one of embodiments 33-39, wherein three three-dimensional electrode comprises gold.

Embodiment 41. The device of any one of embodiments 1-40, wherein each of the plurality of chambers further comprises a medium.

Embodiment 42. The device of embodiment 41, wherein the medium comprises a buffering agent.

Embodiment 43. The device of embodiment 43, wherein the buffering agent is present in the medium at a concentration of from about 21 mM to about 24 mM.

Embodiment 44. The device of embodiment 43, wherein the buffering agent comprises HEPES.

Embodiment 45. The device of embodiment 41-44, wherein the medium comprises an antioxidant.

Embodiment 46. The device of embodiment 45, wherein the antioxidant is present in the medium at a concentration from about 0.00001 millimolar (mM) to about 0.1 mM.

Embodiment 47. The device of embodiment 45, wherein the antioxidant is an amino acid antioxidant.

Embodiment 48. The device of embodiment 47, wherein the amino acid antioxidant is 1-carnosine, 1-carnitine, or a combination thereof.

Embodiment 49. The device of any one of embodiments 41-48, wherein the medium maintains one or more in-vivo-like functions of the cell.

Embodiment 50. The device of embodiment 49, wherein the cell is a neuron and wherein the one or more in-vivo-like functions comprises synaptic function, action potential generation, energetic maintenance, or any combination thereof.

Embodiment 51. The device of any one of embodiments 41-50, wherein the device is placed into an environment.

Embodiment 52. The device of any one of embodiments 51, wherein the environment comprises one or more of (i) a temperature outside of a temperature range of from about 34 degrees C. to about 40 degrees C.; (ii) a humidity outside of a humidity range of from about 75% to about 85% humidity; (iii) a carbon dioxide percentage outside of a carbon dioxide percentage range of from about 3% to about 8%; or (iv) any combination thereof.

Embodiment 53. The device of any one of embodiments 51-52, wherein the medium maintains a pH of from about 7.3 to about 7.5 in the environment for a time period.

Embodiment 54. The device of embodiment 53, wherein the time period is at least about 1 month.

Embodiment 55. The device of any one of embodiments 51-54, wherein the medium maintain an osmolality of from about 280 to about 330 Osm/L in the environment for a time period.

Embodiment 56. The device of embodiment 55, wherein the time period is at least about 1 month.

Embodiment 57. The device of any one of embodiments 51-56, wherein the medium extends survival of the cell in the environment as compared to a cell in the environment the absence of the medium.

Embodiment 58. The device of embodiment 57, wherein an extended survival of the cell is at least about 1 month.

Embodiment 59. A method of detecting a presence or an absence of one or more compounds in an environment, the method comprising: (a) placing a spatially addressable array in the environment, wherein the spatially addressable army comprises: a plurality of chambers, wherein each chamber of the plurality of chambers comprises: (i) a cell modified to express a unique odorant receptor profile; and (ii) an electrical component configured to measure an electrical signal in the cell; and (b) detecting the presence or the absence of the one or more compounds based the measured electrical signals from the plurality of chambers.

Embodiment 60. The method of embodiment 59, wherein the environment is a residential setting.

Embodiment 61. The method of embodiment 59, wherein the environment is a public space.

Embodiment 62. The method of any one of embodiments 59-61, wherein the environment comprises two or more compounds.

Embodiment 63. The method of any one of embodiments 59-62, wherein the environment comprises one or more of (i) a temperature outside of a temperature range of from about 34 degrees C. to about 40 degrees C., (ii) a humidity outside of a humidity range of from about 75% to about 85% humidity; (iii) a carbon dioxide percentage outside of a carbon dioxide percentage range of from about 3% to about 8%; or (iv) any combination thereof.

Embodiment 64. The method of any one of embodiments 59-63, further comprising communicating a result via a communication medium.

Embodiment 65. The method of embodiment 64, wherein the result comprises the presence or the absence of the one or more compounds.

Embodiment 66. The method of embodiment 64, wherein the result comprises a probability of the presence or the absence of the one or more compounds.

Embodiment 67. The method of embodiment 66, wherein the probability is from about 80% to about 99%.

Embodiment 68. The method of embodiment 66, wherein the probability is at least about 85%.

Embodiment 69. The method of any one of embodiments 59-68, further comprising comparing the signaling pattern to one or more signaling patterns associated with one or more reference compounds.

Embodiment 70. The method of any one of embodiments 59-69, wherein the presence or the absence of the one or more compounds comprises measuring an amount of the one or more compounds.

Embodiment 71. The method of any one of embodiments 59-70, wherein the spatially addressable array comprises greater than three unique odorant receptor profiles.

Embodiment 72. The method of any one of embodiments 59-71, wherein the method detects at least two different types of compounds.

Embodiment 73. The method of any one of embodiments 59-72, wherein the cell detects more than one type of compound.

Embodiment 74. The method of any one of embodiments 59-73, wherein the cell is modified to express at least two unique odorant receptor profiles selected from the greater than two unique odorant receptor profiles.

Embodiment 75. A device comprising: an array, the array comprising a plurality of chambers, wherein each of the plurality of chambers comprises: (i) a cell modified to express a cell receptor having a binding specificity for a compound selected from the group consisting of a neurotoxin, a carcinogen, a chemical weapon, and any combination thereof, and (ii) an electrical component configured to measure one or more signals, wherein the device is configured to detect a presence or an absence of the compound based on one or more signals measured in the array.

Embodiment 76. The device of embodiment 75, wherein the presence or the absence of the compound comprises measuring a level of the compound.

Embodiment 77. The device of embodiment 75 or embodiment 76, wherein the cell receptor is an odorant receptor.

Embodiment 78. The device of any one of embodiments 75-77, wherein the cell comprises a genetic modification

Embodiment 79. The device of any one of embodiments 75-78, wherein the cell is a neuron.

Embodiment 80. The device of any one of embodiments 75-79, wherein the cell is a human cell.

Embodiment 81. The device of any one of embodiments 75-80, wherein the array comprises greater than two unique receptors.

Embodiment 82. The device of any one of embodiments 75-81, wherein the device is configured to detect at least two different types of compounds.

Embodiment 83. The device of any one of embodiments 75-82, wherein the cell detects more than one type of compound.

Embodiment 84. The device of any one of embodiments 75-83, wherein the cell is modified to express at least two unique receptors.

Embodiment 85. The device of any one of embodiments 75-84, wherein the cell receptor comprises OR1A1, mOR106-1, OR51E1, OR10J5, OR51E2, mOR9-1, mOR18-1, mOR272-1, mOR31-1, mOR136-1, mOR185-1, mOR189-1, mOR9-1, mOR162-2, mOR202-37, mOR204-3, mOR204-8, mOR204-11, mOR188-1, mOR256-3, mOR156-5, mOR159-3, mOR160-4, mOR160-5, mOR161-5, mOR161-6, mOR162-2, mOR164-2, mOR202-37, mOR204-3, mOR204-8, mOR204-11, mOR165-1, mOR178-1, mOR179-1, mOR183-1, mOR185-1, mOR162-1, mOR189-1, any genetic variation thereof; any functionally active fragment thereof or any combination thereof.

Embodiment 86. The device of any one of embodiments 75-84, wherein the cell receptor comprises OR1A1, mOR106-1, OR51E1, OR10J5, OR51E2, mOR9-1, mOR18-1, mOR272-1, mOR31-1, mOR136-1, any genetic variation thereof any functionally active fragment thereof, or any combination thereof.

Embodiment 87. The device of any one of embodiments 75-84, wherein the cell receptor comprises mOR185-1, mOR189-1, mOR9-1, mOR162-2, mOR202-37, mOR204-3, mOR204-8, mOR204-11, mOR188-1, mOR256-3, any genetic variation thereof any human analog thereof; any functionally active fragment thereof or any combination thereof.

Embodiment 88. The device of any one of embodiments 75-84, wherein the cell receptor comprises mOR156-5, mOR159-3, mOR160-4, mOR160-5, mOR161-5, mOR161-6, mOR162-2, mOR164-2, mOR202-37, mOR204-3, mOR204-8, mOR204-11, any genetic variation thereof any human analog thereof any functionally active fragment thereof or any combination thereof.

Embodiment 89. The device of any one of embodiments 75-84, wherein the cell receptor comprises mOR165-1, mOR178-1, mOR179-1, mOR183-1, mOR185-1, mOR162-1, mOR189-1, any genetic variation thereof any human analog thereof any functionally active fragment thereof, or any combination thereof.

Embodiment 90. The device of any one of embodiments 75-84, wherein the cell receptor comprises a receptor from Table 2b, Table 3, Table 4, any genetic variation thereof, any human analog thereof, any functionally active fragment thereof: or any combination thereof.

Embodiment 91. The device of any one of embodiments 75-84, wherein the cell receptor comprises a modification.

Embodiment 92. The device of embodiment 91, wherein the modification comprises one or more of a genetic modification, a methylation modification, a sulfentation modification, a sulfentation modification, a acylation modification, an alkylation modification, a butyrylation modification, a glycosylation modification, a malonylation modification, a hydroxylation modification, an iodination modification, a propionylation modification, or any combination thereof.

Embodiment 93. The device of embodiment 91, wherein the modification comprises an oxidation modification or reduction modification.

Embodiment 94. The device of embodiment 91, wherein the modification comprises a carbohydrate addition, a carbohydrate deletion, or a combination thereof.

Embodiment 95. The device of any one of embodiments 77-96, further comprising a controller.

Embodiment 96. The device of embodiment 95, wherein the controller is configured to (i) receive the one or more signals and (i) determine a result comprising the presence or the absence of the compound.

Embodiment 97. The device of embodiment 96, wherein the controller communicates the result via a communication medium.

Embodiment 98. The device of any one of embodiments 96-97, wherein the result comprises a probability of the presence or the absence of the compound.

Embodiment 99. The device of embodiment 98, wherein the probability is from about 80% to about 99%.

Embodiment 100. The device of embodiment 98, wherein the probability is at least about 85%.

Embodiment 101. The device of any one of embodiments 95-100, wherein the controller stores one or more signals associated with one or more reference compounds in a database.

Embodiment 102. The device of any one of embodiments 75-101, wherein the one or more signals comprise an action potential, a cell membrane depolarization, an excited signal level that is below a threshold for an action potential, or any combination thereof.

Embodiment 103. The device of any one of embodiments 75-102, wherein the one or more signals comprise a pattern of electrical signals.

Embodiment 104. The device of embodiment 103, wherein the pattern of electrical signals comprises a compound-specific pattern.

Embodiment 105. The device of embodiment 103, wherein the pattern of electrical signals comprises a binary pattern.

Embodiment 106. The device of embodiments 103, wherein the pattern of electrical signals comprises a magnitude of an electrical signal, a temporal pattern of an electrical signal, or a combination thereof.

Embodiment 107. The device of any one of embodiments 75-106, wherein the electrical component comprises an electrode.

Embodiment 108. The device of embodiment 107, wherein the electrode is a three-dimensional electrode.

Embodiment 109. The device of embodiment 108, wherein the three-dimensional electrode comprises a support portion and a head portion.

Embodiment 110. The device of embodiment 109, wherein the head portion comprises a protrusion, a recess, or a combination thereof.

Embodiment 111. The device of embodiment 109, wherein the protrusion comprises a hemispherical protrusion.

Embodiment 112. The device of embodiment 109, wherein the recess comprises a hemispherical recess.

Embodiment 113. The device of any one of embodiments 108-112, wherein the three-dimensional electrode comprises at least 100 protrusions, at least 100 recesses, or any combination thereof.

Embodiment 114. The device of any one of embodiments 108-113, wherein the three-dimensional electrode comprises a surface roughness from about 10 nanometers (nm) to about 100 nm.

Embodiment 115. The device of any one of embodiments 108-114, wherein three three-dimensional electrode comprises gold.

Embodiment 116, the device of any one of embodiments 75-115, wherein the device is placed into an environment.

Embodiment 117. The device of embodiment 116, wherein the environment comprises one or more of (i) a temperature outside of a temperature range of from about 34 degrees C. to about 40 degrees C.; (ii) a humidity outside of a humidity range of from about 75% to about 85% humidity; (iii) a carbon dioxide percentage outside of a carbon dioxide percentage range of from about 3% to about 8%; or (iv) any combination thereof.

Embodiment 118. The device of any one of embodiments 75-117, wherein each of the plurality of chambers further comprises a medium.

Embodiment 119. The device of embodiment 118, wherein the medium comprises a buffering agent.

Embodiment 120. The device of embodiment 119, wherein the buffering agent is present in the medium at a concentration of from about 21 mM to about 24 mM.

Embodiment 121. The device of embodiment 119, wherein the buffering agent comprises HEPES.

Embodiment 122. The device of embodiment 118-121, wherein the medium comprises an antioxidant.

Embodiment 123. The device of embodiment 122, wherein the antioxidant is present in the medium at a concentration from about 0.00001 mM to about 0.1 mM.

Embodiment 124. The device of embodiment 122, wherein the antioxidant is an amino acid antioxidant.

Embodiment 125. The device of embodiment 124, wherein the amino acid antioxidant is l-carnosine, 1-carnitine, or a combination thereof.

Embodiment 126. The device of any one of embodiments 118-125, wherein the medium maintains one or more in-vivo-like functions of the cell.

Embodiment 127. The device of embodiment 126, wherein the cell is a neuron and wherein the one or more in-vivo-like functions comprises synaptic function, action potential generation, energetic maintenance, or any combination thereof.

Embodiment 128. The device of any one of embodiments 118-127, wherein the medium maintains a pH of from about 7.3 to about 7.5 in the environment for a time period.

Embodiment 129. The device of embodiment 128, wherein the time period is at least about 1 month.

Embodiment 130. The device of any one of embodiments 118-129, wherein the medium maintain an osmolality of from about 280 to about 330 Osm/L in the environment for a time period.

Embodiment 131. The device of embodiment 130, wherein the time period is at least about 1 month.

Embodiment 132. The device of any one of embodiments 118-131, wherein the medium extends survival of the cell in the environment as compared to a cell in the environment the absence of the medium.

Embodiment 133. The device of embodiment 132, wherein an extended survival of the cell is at least about 1 month.

Embodiment 134. The device of any one of embodiments 75-133, wherein the compound comprises an illegal substance as defined in 42 United States Code § 12210.

Embodiment 135. The device of any one of embodiments 75-134, wherein the compound is the chemical weapon, and wherein the chemical weapon is a mustard gas, a sarin gas, or any combination thereof.

Embodiment 136. The device of any one of embodiments 75-135, wherein the compound is the carcinogen, and wherein the carcinogen comprises a thyomethane, a hydrocarbon, an oxygen, a carbon dioxide, or any combination thereof.

Embodiment 137. A method of detecting a presence or an absence of a compound in an environment, the method comprising: (a) placing a device in the environment, wherein the device comprises: an array, the array comprising a plurality of chambers, wherein each of the plurality of chambers comprises: (i) a ceil modified to express a cell-surface receptor having a binding specificity for the compound, wherein the compound is selected from the group consisting of a neurotoxin, a carcinogen, a chemical weapon, and any combination thereof, and (ii) an electrical component configured to measure one or more signals; and (b) detecting the presence or the absence of the compound based on one or more signals measured in the device.

Embodiment 138. The method of embodiment 137, wherein the environment is a residential setting.

Embodiment 139. The method of embodiment 137, wherein the environment is a public space.

Embodiment 140. The method of any one of embodiments 137-139, wherein the environment comprises two or more compounds.

Embodiment 141. The method of embodiment 137, wherein the environment comprises one or more of (i) a temperature outside of a temperature range of from about 34 degrees C. to about 40 degrees C.; (ii) a humidity outside of a humidity range of from about 75% to about 85% humidity; (iii) a carbon dioxide percentage outside of a carbon dioxide percentage range of from about 3% to about 8%; or (iv) any combination thereof.

Embodiment 142. The method of any one of embodiments 137-141, further comprising communicating a result via a communication medium.

Embodiment 143. The method of embodiment 142, wherein the result comprises the presence or the absence of the compound.

Embodiment 144. The method of embodiment 142, wherein the result comprises a probability of the presence or the absence of the compound.

Embodiment 145. The method of embodiment 144, wherein the probability is from about 80% to about 99%.

Embodiment 146. The method of embodiment 144, wherein the probability is at least about 85%.

Embodiment 147. The method of any one of embodiments 139-148, wherein the detecting is at a concentration detection limit of about 10 millimolar (mM) or less.

Embodiment 148. The method of embodiment 147, wherein the concentration detection limit is about 10 micromolar (uM) or less.

Embodiment 149. The method of any one of embodiments 137-148, further comprising comparing the one or more signals to one or more signals associated with one or more reference compounds.

Embodiment 150. The method of any one of embodiments 137-149, wherein the presence or the absence of the compound comprises measuring a level of the compound.

Embodiment 151. The method of any one of embodiments 137-150, wherein the array comprises greater than two unique receptors.

Embodiment 152. The method of any one of embodiments 137-151, wherein the method detects at least two different types of compounds.

Embodiment 153. The method of any one of embodiments 137-152, wherein the cell detects more than one type of compound.

Embodiment 154. The method of any one of embodiments 137-153, wherein the cell is modified to express at least two unique receptors.

Embodiment 155. The method of any one of embodiments 137-154, wherein the compound comprises an illegal substance as defined in 42 United States Code § 12210.

Embodiment 156. The method of any one of embodiments 137-155, wherein the compound is the chemical weapon, and wherein the chemical weapon is a mustard gas, a sarin gas, or any combination thereof.

Embodiment 157. The method of any one of embodiments 137-156, wherein the compound is the carcinogen, and wherein the carcinogen comprises a thyomethane, a hydrocarbon, an oxygen, a carbon dioxide, or any combination thereof.

Embodiment 158. An array of n different cells, each of which expresses a unique odorant receptor, wherein the array is capable of detecting greater than n different compounds each at a confidence level greater than about 70%.

Embodiment 159. The array of embodiment 158, wherein n=3 or more.

Embodiment 160. The array of embodiment 158, wherein the different compounds comprise a volatile organic compound.

Embodiment 161. The array of embodiment 158, wherein the confidence level is greater than about 85%.

Embodiment 162. The array of embodiment 158, wherein the array is capable of measuring one or more electrical signals.

Embodiment 163. The array of embodiment 162, wherein the one or more electrical signals comprise an action potential, a membrane depolarization, an excited signal level that is below a threshold for an action potential, or any combination thereof.

### Example 1

Although neurons expressing different olfactory receptors can act as primary sensors for odor recognition when coupled to electrodes, the signatures of the different compounds may be embedded into complex spatiotemporal patterns of electrical activity across the different electrodes present in the system. This may be the result of different neurons reacting with different affinities to the compounds present in the air around them, as well as the fact that each electrode may be in electrical proximity to multiple neurons, and accumulating the signals from each of them.

The mapping from spike patterns to odor detection is challenging for several reasons including one or more of the following:
a. A single neuron may express different types of receptors.
b. Different neurons may express a receptor in different quantity.
c. Receptors may be broadly tuned (such as they can respond equally well to different chemicals).
d. Cells may be in communication with one another—such as neurons that may be connected via excitatory or inhibitory synapses to other neurons, causing a secondary unpredictable response to one or more odorants in the network.
e. Smells, environments, or other sources for detection may be composed of hundreds of different molecules.

Although the consequences of many of the above points may be avoided or limited with appropriate experimental consideration, even in the worst case scenario, different odorants may be classified from simulated data through a supervised learning algorithm that may receive as input the raw neuronal activity and may output the most likely compound or smell that elicited it with sufficient accuracy. In fact, some of the above points may be exploited to improve detection robustness overall (such as sensitivity or specificity), for example, the ability of broadly tuned receptors to respond to different compounds to different degrees.

This demonstration to classify different odorants is done in 3 parts:
a. Realistic simulation of electrophysiological properties of olfactory neurons.
b. Simulation of realistic olfactory responses of neural networks expressing a mixture of olfactory receptors.
c. Training of ML algorithm to discriminate between different patterns of evoked electrical activity.

Realistic Simulation of Electrophysiological Properties of Olfactory Neurons.

To understand this argument from a bottom up approach, it may be important to examine and understand the internal cellular cascade that generates action potentials as a result of odorant binding, especially how odors binding to different receptors, or receptors with broad affinity for many environmental stimuli, can affect the spiking behavior of these cells and networks. Such broad affinity receptors may have the potential to cause false positive detection results, however, this section will demonstrate through single cell computational models that even broadly tuned receptors can modify spiking behavior in a distinguishable manner.

In the task of identifying a particular odorant using a neural-cell based biosensor, it may be important to address the problem of false positives. Due to the nature of receptor biology, some receptors may be broadly or narrowly tuned to different classes of ligands. That is, while some receptors may have extremely specific interactions with only one ligand, and bind to only one particular chemical, many receptors may be more broadly tuned. These broadly tuned receptors may respond very strongly to one ligand, but they may also have weaker interactions with similar, but altogether different, molecules. Because of this phenomenon, there may be off-target interactions wherein the receptor may respond as though the compound of interest had been detected, but it may be in reality a structurally similar, but different chemical. Because the interaction of ligands with receptors may be defined by the electrostatic interaction of the molecule with the binding domain of the protein, the affinity of the molecule for the receptor may necessarily be different for a different molecular structure. How much affinity a receptor has for a certain kind of compound may be defined by the dissociation constant, Kd, which may be defined as the propensity of the protein-ligand complex to separate (dissociate) reversibly back into just the receptor and just the ligand alone.

Based on data as described herein, compounds with different affinity for a receptor of interest may result in significantly different levels of cAMP-associated luminescence in a cell-based assay. The question remains if this difference may be preserved at the level of the action potential, or spike burst, level of cellular response. A mathematical model developed by Dougherty et al. (Dougherty D P, Wright G A, Yew A C. Computational model of the cAMP-mediated sensory response and calcium-dependent adaptation in vertebrate olfactory receptor neurons. Proc Natl Acad Sci USA. 2005; 102(30):10415-20) has captured the entire signal transduction cascade from ligand docking, through G protein activation and the eventual generation of currents from cyclic nucleotide-gated channels which eventually may result in action potentials, a detection readout of devices as described herein. This model may capture the kinds of adaptation and desensitization behaviors that may have been observed in patch-clamp recordings of neurons exposed to varying concentrations of odorants, so it may be a reliable proxy for the internal cellular pathways that determine how a cell responds to odorant stimulation. The question may be, does a lower, or off-target, binding event (such as a false positive) result in a noticeable change in the responsive spike pattern, and if so, may these two spike trains be reliably discriminated? Data described herein may show that not only can this complex be modeled but also the response resulting from ligands can be classified with greater or lesser affinity for the receptor algorithmically.

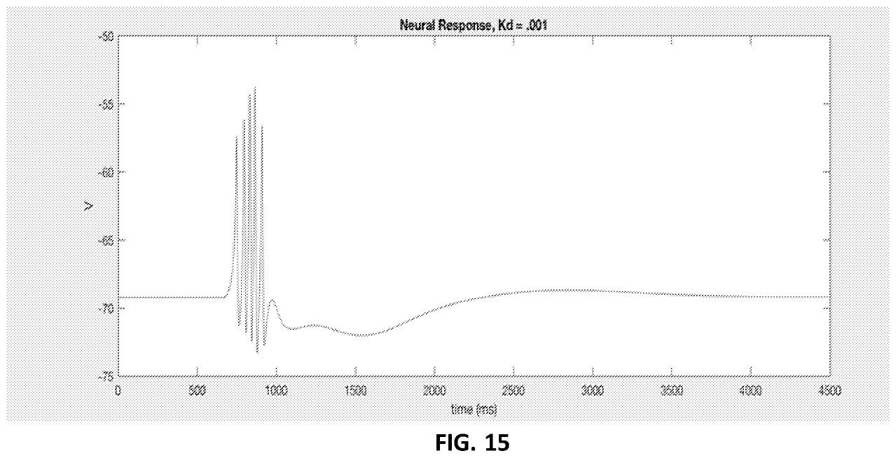

The trace in FIG. 15 represents the spiking behavior of a model neuron expressing a model odorant receptor with a low defined dissociation constant for a ligand exposed to the cell for a 50 millisecond (ms) pulse. A low dissociation constant, as mentioned, means that the ligand may have a high affinity for the receptor and may activate it and the related downstream signaling cascade for a comparatively long time. Due to the low dissociation constant, a substantial spike burst with a 500 ms total duration results.

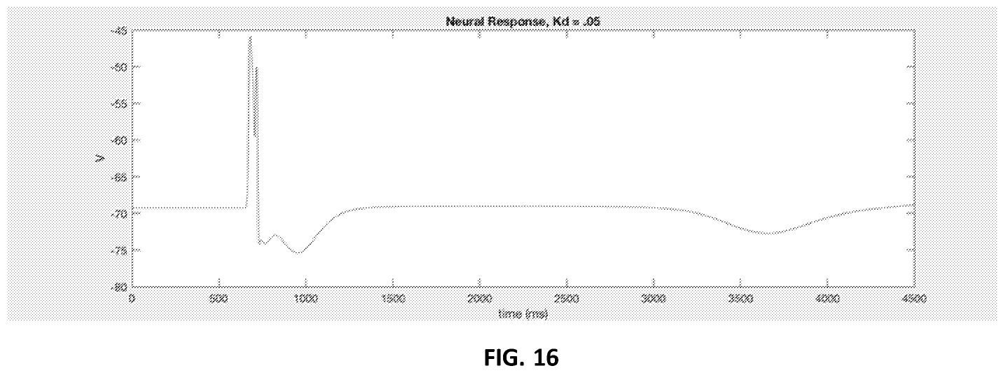

Conversely, the trace in FIG. 16 results from the exact same model neuron, but exposed to a 50 ms pulse of a ligand for which the receptor may have a high defined dissociation coefficient, meaning that the ligand may be likely to separate from the receptor often, resulting in less activation of the downstream signaling cascade. This results in fewer action potentials and a shorter spike burst of only around 100 ms.

Based on these simulation results, there may be clear differences between the spiking behavior resulting from ligand-receptor complexes of different affinities. Therefore, even on a single cell level, off-target detection events characterized by ligand-receptor interactions with higher dissociation coefficients may be measurably different from those narrower, on-target interactions that characterize an actual, true positive detection event.

Simulation of Realistic Olfactory Responses of Neural Networks Expressing a Mixture of Olfactory Receptors.

In the event that the readout may be in actuality the net response of an entire population of cells, a different and perhaps even more robust tactic can be employed to discriminate the neural responses to different odorants. In such a population of neurons, there may be be a number of different receptors expressed on the surface of each cell. If these receptors may be all broadly tuned for the odorant in question, they may each engender a different level of response to the same chemical stimulus. For example, receptor A, expressed in one cell type, may bind the odorant with 50% affinity, receptor B with 30% affinity, and receptor C with 80% affinity. This pattern can be used to uniquely identify the compound from the spiking activity, because as demonstrated herein, different ligand-receptor affinities may result in different spiking behavior in a cell expressing that receptor. In some cases, a similar compound, with a similar chemical structure, which may generate an off-target response in these receptors, may activate each receptor at a different level (receptor A with 20% affinity, receptor B with 80% affinity, and receptor C with 80% affinity), therefore producing a completely different activity pattern, and it may therefore be distinguished from the actual chemical of interest by examining the network behavior overall.

From a more global perspective, in an interconnected network of neurons, each cell may express the odorant receptors of interest at different levels, with some cells having very high levels of expression and many cells no expression at all. For this reason, it may be important to examine if and how the few neurons with a high enough level of expression of the correct receptor to respond to the chemical of interest may impact the overall network activity. For example, if the remainder of the neurons respond unpredictably and for extended intervals to a provoked odor stimulus, then detection events may become difficult. The computational simulation below demonstrates that the few responsive neurons may not unpredictably perturb the overall network activity to a degree that would make proper odorant identification and classification unfeasible.

To see the impact of “odorant-stimulated” neurons at the network scale, a network of 500 Izhikevich spiking neurons [Izhikevich (2003)] were created in Python. The Izhikevich model is a set of coupled differential equations which model biophysically realistic spiking neural behavior. The neurons connect to one another via randomly distributed variable weight synapses. Additionally, a Gaussian noise was introduced to a random fraction of the network for the duration of the experiment. This simulates random odorant-associated stimulation of the small fraction of the network which is transfected with a receptor at an appropriately high level to generate spiking behavior. To interpret experiments, raster plots are used, with the time step in the x-axes and the neuron number that has fired in y-axes.

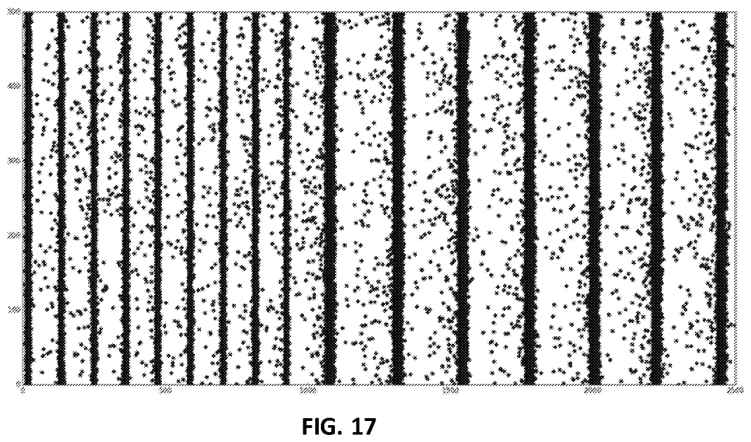

As shown in FIG. 17, a baseline experiment is first run to see the impact of the gaussian noise introduced to the random fraction of the network. Interestingly, the frequency of burst may be reduced at mid-simulation. This may be probably due to the intrinsic properties of the synapses as coded into the model.

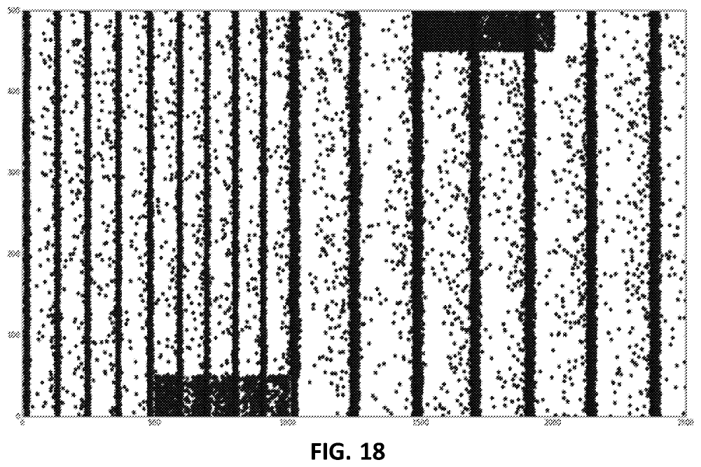

Experiment 1 as shown in FIG. 18, demonstrates that even with a certain level of background noise, a signal for each of the two narrowly tuned receptors is distinguishable. The first experiment utilized 2500 time steps for solving the associated model spiking equations. The network is comprised of two subpopulations of neurons with the following characteristics: (a) Subpopulation 1 has an affinity of 100% for odorant A and 0% for odorant B; (b) Subpopulation 2 has an affinity of 0%0 for odorant A and 100% for odorant B. (Note: on the raster plot of FIG. 18, Subpopulation 1 is the 50 first neurons at the bottom of the plot, and Subpopulation 2 is the last 50 neurons at the top of the plot). In the raster plot of FIG. 18, the following time course of stimulation was applied to the network (time on the x axis): 0-500: No odorant; 500-1000: release of odorant A; 1000-1500: No odorant; 1500-2000: release of odorant B; 2000-2500: No odorant.

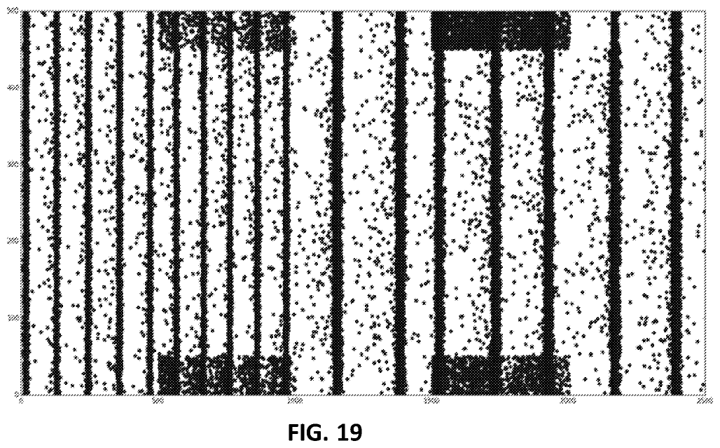

Experiment 2 as shown in FIG. 19 demonstrates that even with two different broadly tuned receptors, the two can still be distinguished based on a measured electrical output (wherein the distinguishable different may be based on a difference in density of a given region on a raster plot). The second experiment also utilized 2500 time steps, with two subpopulations of neurons that have the following characteristics: (a) Subpopulation 1 has an affinity of 70% for odorant A and 30% for odorant B; (b) Subpopulation 2 has an affinity of 30% for odorant A and 70% for odorant B. In the raster plot of FIG. 19, the following time course of stimulation was applied to the network (time on the x axis): 0-500: No odorant; 500-1000: release of odorant A; 1000-1500: No odorant; 1500-2000: release of odorant B; 2000-2500: No odorant.

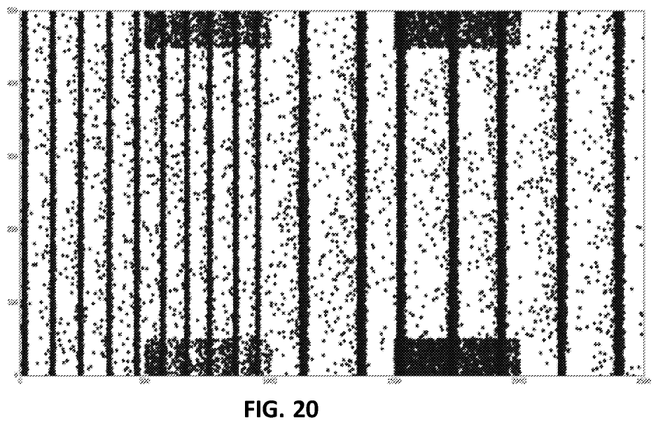

In the raster plot of FIG. 20, the following time course of stimulation was applied to the network (time on the x axis): 0-500: No odorant; 500-1000: release of odorant B: 1000-1500: No odorant; 1500-2000: release of odorant A; 2000-2500 No odorant.

In spite of the connectivity within the network, only the neurons that are directly stimulated may significantly increase their firing frequency. Indeed, these experiments may demonstrate that at a large time scale, the increase in burst frequency and duration in the rest of the network upon stimulation may be small as compared to baseline. This may mean that the recorded evoked activity may be necessarily a result of neurons directly responding to the odorant in question, not random extraneous network perturbations which may result in a potential false positive. In some cases, classification of a stimulus may occur without direct access to the signal of the neurons that may be directly responding. This situation may arise if the cultured neurons transfected with the appropriate odorant receptor are physically distant from the recording electrodes.

These simulations may demonstrate that in networks of subpopulations of neurons randomly modified, as the case would may be in a “batch” genetically modified neural network, with receptors with varying degrees of affinity for two different odorants, can respond in a distinguishable manner to a time course of stimulation with the two odorants in question, showing that even in a worst-case of uncontrolled levels of receptor expression and off-target affinity, similar odorants can still be reliably discriminated.

### Example 2

Training a Machine Learning (ML) Algorithm to Discriminate Between Different Pattern of Evoked Electrical Activity.

As described herein, for example Example 1, single neuron and network electrical activity may differ as a result of exposure to different compounds in the environment. Classifiers may be developed to reliably distinguish these different detection cases and to discriminate the true positive results from the false positives generated by structurally similar chemicals which may result in the aforementioned off-target partial receptor activation.

Different experiments that may correspond to different real life situation may be considered, for example, (a) neurons that may be directly responding to an odorant, or (b) only neurons that may be responding as a result of their synaptic connections to directly stimulated neurons. The two cases may be distinguished, (i) one case where the neurons may respond very specifically to either compound, the (ii) other case where they only have a moderate bias towards one or the other analyte—because of overlapping expression, or because of broadly tuned receptors.

Protocol (for Experiments 1, 2, 3 as Described Below):

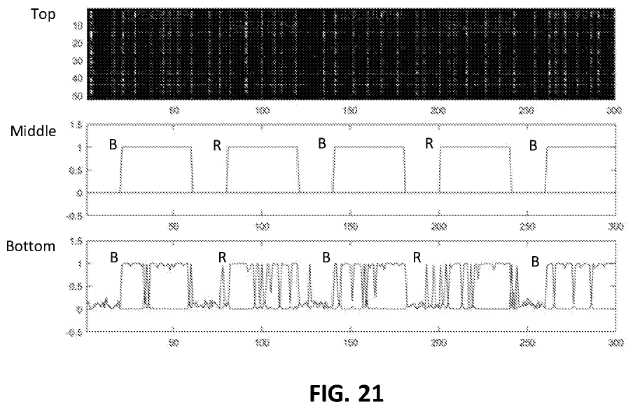

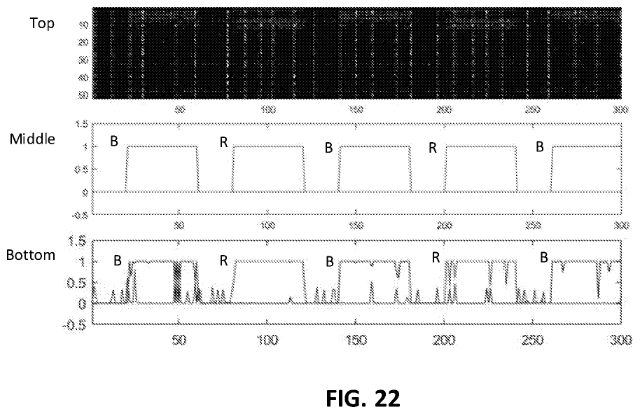

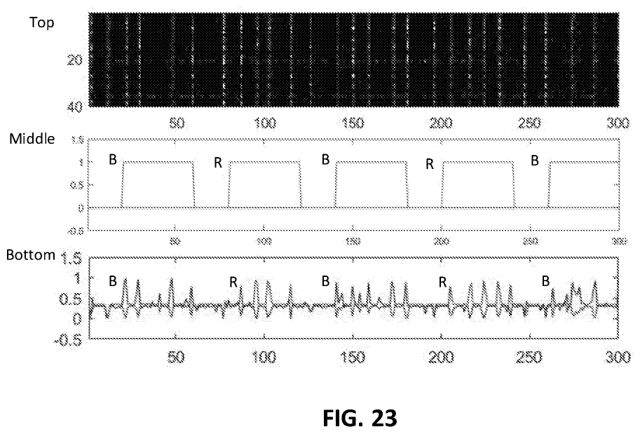

A small population of 50 neurons is generated, 2 subpopulations of 5 neurons each (10% of the total size of the network) are modified each to respond preferentially to chemical A or to chemical B. The network is simulated, and the number of spikes for each neuron is summed using a bin size of 50 ms (see top panels of FIG. 21, FIG. 22, and FIG. 23). Periodically, chemical A, chemical B, or no chemical are introduced, causing the subpopulations to respond accordingly to the receptor they express (and their respective affinity for compound A or B).

Each bin is labelled with “chemical A” or “chemical B” or “nothing” accordingly to the chemical present at that time, creating a whole data set used for training and validation. The whole simulation consists in 100 trials of alternating 2 second periods of exposure to A or B, with 1 second rinse in between. The bins of the first 70 stimuli are used as training dataset for the machine learning classifier, and the last 30 for validation. This represents a total of 2/0.05×70=2800 training example for chemical “A”, and another 2800 for chemical “B”, and 1/0.05*140=2800 for “baseline”, or no chemical at all.

The data is used to train an artificial neural network with an input vector size of N×S, where N is the number of biological neurons stimulated in the culture, or just a subset of these neurons, and s is the number of consecutive time bins that the artificial neural network sees at once, 500 hidden units, and 2 output units, corresponding to A and B. Neurons in the hidden and output layer have a sigmoid activation function. Training is done using backpropagation to compute the gradient and adadelta, a gradient descent optimization algorithm, is used to update the weights.

Results:

### Experiment 1

The receptors are narrowly tuned (responding 100% to compound A and 0% to B) and the signals are directly recorded from the cells having the narrowly tuned receptors (i.e. the neurons responding directly are “visible” in the input data, meaning that we can directly record electrical information from the neurons transected with the odorant receptor tuned to respond to the odor stimulus.) The x axis is time and the y axis is a probability of predicting a compound or odorant. The top panel shows raw data in a raster plot. The middle panel shows the experimental conditions or experimental truth. The bottom panel shows the predicted outcome.

As shown in FIG. 21, odorant classification from complex activity patterns in Experiment 1. Top panel: a binned raster plot showing 15 seconds of activity (x-axis) over 50 neurons (y-axis). Middle panel: Chemical stimulation of “A” marked as “B” and “B” marked as “R” from the raster plot above. The subset receives an odorant current of 10 or 0 when presented molecule A and B respectively. The opposite for the second subset. Bottom panel: prediction of the classifier after 2000 training epochs, produced from the data presented above. Marked as “B” is the probability that chemical A is present. Marked as “R” is the probability that B is present. The prediction matches the ground truth with good accuracy, which can be seen by comparing the second and third subplots.

### Experiment 2

The receptors are broadly tuned and the signals are directly recorded from the cells having the broadly tuned receptors. This experiment is designed to prove that good network odorant classification may still be possible in the case of broadly tuned receptors, i.e., the case where a receptor can respond to both chemical stimuli at different degrees. The receptors are broadly tuned (responding 75% to compound A and 25% to B), and the neurons responding directly are again “visible” in the input data. The x axis is time and the y axis is a probability of predicting a compound or odorant. The top panel shows raw data in a raster plot. The middle panel shows the experimental conditions or experimental truth. The bottom panel shows the predicted outcome.

As shown in FIG. 22, odorant classification from complex activity patterns in Experiment 2. Top panel: a binned raster plot showing 15 seconds of activity over 50 neurons. Middle panel Chemical stimulation of “A” marked as “B” and “B” marked as “R” from the raster plot above. The first subset receives a current of 15 and 5 when presented molecule A and B respectively, to model the broadly tuned receptor effect. The opposite is true for the second subset. Bottom panel, prediction of the classifier after 2000 training epochs, produced from the data presented above. Marked as “B” is the probability that chemical A is present. Marked as “R” is the probability that B is present. The prediction matches the ground truth with good accuracy.

### Experiment 3

The receptors are broadly tuned and signaled are recorded from another cell in communication with the cell having the broadly tuned receptor (such as a secondary neuron that is contacting an electrode and in communication with the primary neuron having the broadly tuned receptor). The receptors are broadly tuned (75/25), and the neurons responding directly are “invisible” in the input data, meaning that the recorded electrical response is due to the activity of secondary neurons connected to the responsive neurons synaptically. The x axis is time and the y axis is a probability of predicting a compound or odorant. The top panel shows raw data in a raster plot. The middle panel shows the experimental conditions or experimental truth. The bottom panel shows the predicted outcome.

As shown in FIG. 23, odorant classification from complex activity patterns in Experiment 3. Top panel: a binned raster plot showing 15 seconds of activity over 50 neurons. Middle panel: Chemical stimulation of “A” marked as “B” and “B” marked as “R” from the raster plot above. The first subset receives a current of 15 and 5 when presented molecule A and B respectively, to model the broadly tuned receptor effect. The opposite is true for the second subset. Bottom panel prediction of the classifier after 2000 training epochs, produced from the data presented above. Marked as “B” is the probability that chemical A is present. Marked as “R” is the probability that B is present. The prediction matches the ground truth with good accuracy.

Discussion:

As described herein, it may be demonstrated that for a broadly tuned receptor with a varying affinity for two different odorants, A and B, this difference in ligand-receptor dissociation may directly appear in the downstream action potential readout, therefore making odorant discrimination possible at the single-cell level based on a computer simulation modeling the entire olfactory signaling cascade, as shown in FIG. 15 and FIG. 16, wherein each figures demonstrates a detectable and distinguishable signal output.

In Example 1, it may be demonstrated that in a randomly transfected network of neurons on a clip, the neurons transfected with the ability to detect an odor, A or B, may respond far above the network baseline, making detection events possible as confounding aberrant noise from the rest of the network may be negligible, as shown in FIG. 17-20.

In Example 2, the efficacy of a machine learning classifier, in this case an ANN, may be demonstrated in discriminating between two odorants presented to the network. As shown in FIG. 21, when receptors are narrowly tuned and the neurons responding directly are part of the recording, a modest ANN may be capable of quickly learning to discriminate between two odorants, even though there may be no striking difference in global activity in the raster plot. As shown in FIG. 22, even with broadly tuned receptor preferentially responding 75% for chemical A and 25% for chemical B, and vice-versa, the network can still learn to classify between the two odorants. This is a surprising an unexpected result. The 75-25 ratio may be represented in the model by a current injected upon stimulation with either odorant and not the firing rate.

Further, as shown in FIG. 23, even if the receptors are broadly tuned and even if lacking in the ability to directly record from the neurons that are responding directly to odorant stimulations, (for instance if the electrodes are not directly contacting the modified neurons), the activity that propagates in the rest of the network may be sufficient to discriminate between the two odorants. This is also a surprising and unexpected result. In some cases, learning of the ANN may take longer (because the features to be extracted may be less obvious, as intuitively understood from the raster plot).

Advantages of arrays, devices, and systems as described herein may include (a) the ability to discriminate the presence of two or more compounds using electrical signals from secondary cells that are in communication with primary cells having the odorant receptors that can bind to the two or more compounds, (b) the ability of a device having two or more broadly tuned receptors to individually detect a presence or an absence of two or more compounds, or (c) a combination thereof.

### Example 3

An array will be configured with 3 different cells, each cell of which will express a unique odorant receptor. The array will be capable of detecting greater than 3 different compounds, each at a confidence level greater than about 85%.

### Example 4

An environment will be diagnosed for the presence or absence of a panel of volatile organic compounds. An array will be placed into the environment. The array will detect the presence or the absence of each of the volatile organic compounds at an accuracy of at least about 85% and with a detection threshold of 10 pM for each of the volatile organic compounds.

### Example 5

An array will be configured with nine chambers. The first three chambers (a00, a01, a02) will each comprises a cell having a broadly tuned receptor for DNT. The second three chambers (a10, a11, a12) will each comprise a cell having a narrowly tuned receptor for DNT.

The last three chambers (a20, a21, a22) will each comprise a cell having a moderately tuned receptor for DNT. Each cell will contact an electrode. The array will be placed into an environment in which DNT will be present. The cells in the second three chambers will produce an electrical signal comprising a full action potential in response to the DNT and given a value of 1.0. The cells in the last three chambers will produce an electrical signal comprising a subthreshold membrane depolarization in response to the DNT and given a value of 0.5. The cells in the first three chambers will produce an electrical signal comprising a subthreshold membrane depolarization in response to the DNT and given a value of 0.1. A matrix of electrical signals will be formulated as follows:

$$\begin{matrix}a 00 , a 01 , a 02 \\ a 10 , a 11 , a 12 \\ a 20 , a 21 , a 22\end{matrix} = \begin{matrix}0.1 , 0.1 , 0.1 \\ 1.0 , 1.0 , 1.0 \\ 0.5 , 0.5 , 0.5\end{matrix}$$

The matrix of signals will be a uniquely identifying fingerprint for that panel of receptors detecting that sample comprising DNT

### Example 6

An array will be configured with nine chambers. The first three chambers will each comprise a cell having a broadly tuned receptor for DNT. The second three chambers will each comprise a cell having a narrowly tuned receptor for DNT. The last three chambers will each comprise a cell having a moderately tuned receptor for DNT. Each cell will contact an electrode. The array will be placed into an environment in which DNT will be present. A second array having nine chambers will also be placed into the environment in which DNT will be present. All nine chambers of the second array will comprise a cell having the broadly tuned receptor for DNT. An electrical pattern of signaling will be measured in each chamber of both arrays. The sensitivity of detection of the DNT in the array having the broadly, moderately, and narrowly tuned receptors will be 20% greater than the sensitivity of detection of the DNT in the array having only broadly tuned receptors.

### Example 7

An array will be configured with 30 chambers. The first 10 chambers will each comprise a cell having a broadly tuned receptor for vanillic acid. The second 10 chambers will each comprise a cell having a narrowly tuned receptor for vanillic acid (such as mOR9-1). The last 10 chambers will each comprise a cell having a moderately tuned receptor for vanillic acid. Each cell will contact an electrode. A sample comprising 10 pM of vanillic acid will be placed into each chamber of the array. A second array having 30 chambers will also receive the sample comprising vanillic acid in each chamber of the second array. All 30 chambers of the second array will comprise a cell having the broadly tuned receptor for vanillic acid. An electrical pattern of signaling will be measured in each chamber of both arrays. The array having the broadly, moderately, and narrowly tuned receptors will be able to detect the presence of the 10 pM vanillic acid sample as compared to the array having only the broadly tuned receptors which will not be able to detect the 10 pM of vanillic acid.

### Example 8

An array will be configured with 6 chambers. The first 3 chambers will comprise a cell having a receptor broadly tuned to cocaine (such as mOR18-1) and narrowly tuned to heroine. The second 3 chambers will comprise a cell having a receptor broadly tuned to heroine and narrowly tuned to cocaine. When sample A having heroine is contacted to the chambers of the array a first unique pattern of electrical signals will be produced. When sample B having cocaine is contacted to the chambers of the array a second unique pattern of electrical signals will be produced. When sample C having both cocaine and heroine is contacted to the chambers of the array a third unique pattern of electrical signals will be produced. Each of the 1^{st}, 2^{nd}, ad 3^{rd} unique patterns will be distinguishable from one another.

### Example 9

### The Micro-Electrode Array (MEA) for Detecting a Range of Odorants, Representing a State, Such as a Ripeness State of a Single Piece of Fruit or a Batch of Fruit.

Table 2a comprises a list of odorant compounds that are produced by fruit. Table 2b comprises a list of insect odorant receptors that may bind one or more of the compounds in Table 2a.

In some embodiments, each neuronal cell may express multiple copies of a single odorant receptor. For redundancy, different cells may express multiple copies of a given compound receptor and other cells may express other compound receptors. A detection array may consist of cells where each odorant receptor may recognize one or more of the compounds in Table 2a, and thus, can detect a single odorant compound or a mixture of the odorant compounds.

With a sampling device possibly coupled to a non-specific resin-based air concentrator, which can collect an air sample containing some odorant compounds, from near the fruit or seedling, one can transport the air sample so that the detection device may be exposed directly or through a membrane containing liquid media. The odorants may then pass through the membrane or liquid covering to the neurons on the detection device. Upon binding to an odorant receptor, the G-protein pathway may signal inside the cell, may amplify the signal and an action potential may be triggered. Because each cell may have an associated electrode, an electrical impulse may be sent to a signal detector. Each electrode may be wired such that the binding of an odorant to a particular cell may result in a unique signal (based on location in the array) such that the controller may identify which cell has bound odorant.

This system may permit mapping back to the odorant receptor since each cell may uniquely express a single odorant receptor. Through the decoding of odorant receptors, one can obtain a pattern of receptors that have been activated. Thus, the particular set of odorants may yield a particular pattern. This pattern may likely be compound concentration-dependent to some extent.

Furthermore, because the electrodes permit sub-threshold signals, one can derive quantitative information from each cell, yielding some information about odorant concentration. By running standard control samples across the array, one may create a database of how well different compounds bind across the array. Furthermore, for each of these controls, one can perform detection based on a serial dilution curve, which may allow one to map back a concentration from an unknown sample.

That is, the pattern of binding across the array may be more than just a binary output, such as on/off, but may also capture some information about odorant concentration levels. The pattern of action potentials generated by sufficiently excited neurons may also contain information regarding concentration. Thus, one can map back from the results of a test sample and may be able to estimate the concentration of the odorant in the test sample.

In the case of multiple types of odorants binding to multiple cells, one may receive a more complex pattern or fingerprint for the particular mixture since it may also encode concentration information and relative concentration with overlapping effects.

Table 2a as shown below, provides a set of odorant compounds produced by fruit or plants.

TABLE 2a

| Compound Name | CAS # |
|---|---|
| alpha-ionone | 127-41-3 |
| alpha-phellandrene | 99-83-2 |
| alpha-pinene | 7785-70-8 |
| benzaldehyde | 100-52-7 |
| beta-ionone | 14901-07-6 |
| beta-pinene | 18172-67-3 |
| butyric acid | 107-92-6 |
| caryophyllen | 87-44-5 |
| damascenone | 23726-93-4 |
| delta-decalactone | 705-86-2 |
| e-2-hexenal | 6728-26-3 |
| ethyl butyrate | 105-54-4 |
| gamma-decalactone | 706-14-9 |
| geranial | 5392-40-5 |
| geraniol | 106-24-1 |
| hexanoic acid | 142-62-1 |
| hexyl acetate | 142-92-7 |
| limonene | 138-86-3 |
| linalool | 78-70-6 |
| mesifuran | 4077-47-8 |
| methyl anthranilate | 134-20-3 |
| methyl butyrate | 623-42-7 |
| neral | 5392-40-5 |
| nerolidol | 7212-44-4 |
| raspberry ketone | 5471-51-2 |

Table 2b, as shown below, provides a set of odorant receptors for fruit-specific volatile compounds.

TABLE 2b

| | | | Literature | | | |
|---|---|---|---|---|---|---|
| Odorant | CAS# | organism | code | GenBank ID | literture indication | Reference |
| [symbol] | 138-86 [symbol] | [symbol] | [symbol] | [symbol] | [symbol] | [symbol] A Female-Biased |
| | | | | | | Odorant Receptor from [symbol] |
| | | | | | | Tuned to Some Plant Odors [symbol] |
| | | | | | | [symbol] |
| | | | | | | [symbol] |
| [symbol] | 138-86 [symbol] | [symbol] | [symbol] | [symbol] | [symbol] | [symbol] |
| | | | | | | [symbol] |
| | | | | | | [symbol] Crystal Structures and Binding Dynamics of |
| | | | | | | Odorant-Binding Protein 3 from two [symbol] |
| | | | | | | [symbol] |
| | | | | | | [symbol] |
| | | | | | | [symbol] |
| [symbol] | 138-86 [symbol] | [symbol] | [symbol] | [symbol] | [symbol] | [symbol] |
| | | | | | | [symbol] Identification of Host-Plant Volatiles and |
| | | | | | | Characterization of Two Novel General Odorant-Binding |
| | | | | | | Proteins from the [symbol] |
| | | | | | | [symbol] |
| | | | | | | [symbol] |
| | | | | | | [symbol] |
| | | | | | | [symbol] |
| [symbol] | 138-86 [symbol] | [symbol] | [symbol] | [symbol] | [symbol] | [symbol] |
| | | | | | | [symbol] |
| | | | | | | for egg laying on citrus substrates in [symbol] |
| | | | | | | [symbol] |
| | | | | | | [symbol] |
| | | | | | | [symbol] |
| [symbol] | 78-70 [symbol] | [symbol] | [symbol] | [symbol] | [symbol] | [symbol] |
| | | | | | | [symbol] |
| | | | | | | [symbol] |
| | | | | | | [symbol] |
| | | | | | | [symbol] |
| | | | | | | [symbol] |

[symbol]

indicates data missing or illegible when filed

Table 3, as shown below, provides a set of odorant receptors.

TABLE 3

| Gene Name | Accession Number |
|---|---|
| odorant receptor family 7 subfamily D member 4 P79L variant [Homo sapiens] | ABV66285.1 |
| odorant receptor family 7 subfamily D member 4 S84N variant [Homo sapiens] | ABV66284.1 |
| odorant receptor family 7 subfamily D member 4 WM variant [Homo sapiens] | ABV66283.1 |
| odorant receptor family 7 subfamily D member 4 RT variant [Homo sapiens] | ABV66282.1 |
| odorant receptor HOR3′beta5 [Homo sapiens] | AAG42368.1 |
| odorant receptor HOR3′beta4 [Homo sapiens] | AAG42367.1 |
| odorant receptor HOR3′beta3 [Homo sapiens] | AAG42366.1 |
| odorant receptor HOR3′beta2 [Homo sapiens] | AAG42365.1 |
| odorant receptor HOR3′beta1 [Homo sapiens] | AAG42364.1 |
| olfactory receptor 7D4 [Homo sapiens] | NP_001005191.1 |
| HOR 5′Beta1 [Homo sapiens] | AAD29426.2 |
| HOR 5′Beta3 [Homo sapiens] | AAD29425.2 |
| F20722_2 [Homo sapiens] | AAC14389.1 |
| olfactory receptor 2J3 [Homo sapiens] | NP_001005216.2 |
| olfactory receptor 2H1 [Homo sapiens] | NP_001304951.1 |
| olfactory receptor 2H1 [Homo sapiens] | NP_001304943.1 |
| olfactory receptor 2H1 [Homo sapiens] | NP_112145.1 |
| olfactory receptor 11A1 [Homo sapiens] | NP_039225.1 |
| olfactory receptor 51B4 [Homo sapiens] | NP_149419.2 |
| olfactory receptor 51B2 [Homo sapiens] | NP_149420.4 |
| olfactory receptor 2J2 [Homo sapiens] | NP_112167.2 |
| olfactory receptor 2H2 [Homo sapiens] | NP_009091.3 |
| olfactory receptor 10G4 [Homo sapiens] | NP_001004462.1 |
| olfactory receptor 12D2 [Homo sapiens] | NP_039224.2 |
| olfactory receptor 2F1 [Homo sapiens] | NP_036501.2 |
| olfactory receptor 51M1 [Homo sapiens] | NP_001004756.2 |
| olfactory receptor 51I1 [Homo sapiens] | NP_001005288.1 |
| olfactory receptor 52D1 [Homo sapiens] | NP_001005163.1 |
| olfactory receptor 51I2 [Homo sapiens] | NP_001004754.1 |
| olfactory receptor 51B5 [Homo sapiens] | NP_001005567.2 |
| olfactory receptor 3A1 [Homo sapiens] | NP_002541.2 |
| olfactory receptor 51B6 [Homo sapiens] | NP_001004750.1 |
| olfactory receptor 5V1 [Homo sapiens] | NP_110503.3 |
| olfactory receptor 12D3 [Homo sapiens] | NP_112221.1 |
| olfactory receptor 10C1 [Homo sapiens] | NP_039229.3 |
| putative olfactory receptor 2B3 [Homo sapiens] | NP_001005226.1 |
| OR1F12, partial [Homo sapiens] | ADA83722.1 |
| OR12D3, partial [Homo sapiens] | ADA83721.1 |
| OR1F12, partial [Homo sapiens] | ADA83720.1 |
| F20722_1 [Homo sapiens] | AAC14388.1 |
| olfactory receptor [Homo sapiens] | CAD31042.1 |
| olfactory receptor [Homo sapiens] | CAD31041.1 |
| olfactory receptor [Homo sapiens] | CAD31040.1 |
| olfactory receptor [Homo sapiens] | CAD31039.1 |
| olfactory receptor [Homo sapiens] | CAD31038.1 |
| olfactory receptor [Homo sapiens] | CAD31037.1 |
| Olfactory receptor 51B4; Odorant receptor HOR5′beta1 | Q9Y5P0.3 |
| Olfactory receptor 51B2; Odorant receptor HOR5′beta3; Olfactory receptor | Q9Y5P1.4 |
| 51B1 | |
| Olfactory receptor 7D4; OR19-B; Odorant receptor family subfamily D | Q8NG98.1 |
| member 4RT; Olfactory receptor OR19-7 | |
| Olfactory receptor 1D2; Olfactory receptor 17-4; OR17-4; Olfactory receptor | P34982.2 |
| OR17-6; Olfactory receptor-like protein HGMP07E | |
| Olfactory receptor 12D3; Hs6M1-27; Olfactory receptor OR6-27 | Q9UGF7.1 |
| Olfactory receptor 5V1; Hs6M1-21; Olfactory receptor OR6-26 | Q9UGF6.1 |
| Olfactory receptor 11A1; Hs6M1-18; Olfactory receptor 11A2; Olfactory | Q9GZK7.1 |
| receptor OR6-30 | |
| Olfactory receptor 2H1; Hs6M1-16; OLFR42A-9004.14/9026.2; Olfactory | Q9GZK4.1 |
| receptor 2H6; Olfactory receptor 2H8; Olfactory receptor 6-2; OR6-2; | |
| Olfactory receptor OR6-32 | |
| Olfactory receptor 2J3; Hs6M1-3; Olfactory receptor OR6-16; OR6-6; | O76001.1 |
| Olfactory receptor 6-6 | |
| Receptor expression-enhancing protein 1 | Q9H902.1 |
| Receptor expression-enhancing protein 2 | Q9BRK0.2 |
| Olfactory receptor 5H8; Olfactory receptor 5H8 pseudogene; Olfactory | P0DN80.1 |
| receptor OR3-7 | |
| Olfactory receptor 13C7 | P0DN81.1 |
| Olfactory receptor 12D1; Olfactory receptor 12D1 pseudogene | P0DN82.1 |
| Putative olfactory receptor 8G3 pseudogene; Olfactory receptor OR11-297 | P0DMU2.1 |
| Putative olfactory receptor 13C6; Olfactory receptor, family 13, subfamily C, | Q8NH95.2 |
| member 6 pseudogene; Olfactory receptor, family 13, subfamily C, member 7 | |
| pseudogene; Putative olfactory receptor 13C7 | |
| Olfactory receptor 8G5; Olfactory receptor 8G6; Olfactory receptor OR11-298 | Q8NG78.2 |
| Olfactory receptor 51M1; Odorant receptor HOR5′beta7; Olfactory receptor | Q9H341.4 |
| OR11-40 | |
| Olfactory receptor 52E5 327 aa protein | Q8NH55.2 |
| Olfactory receptor 4A5; Olfactory receptor OR11-111 | Q8NH83.4 |
| Olfactory receptor 5K1; HTPCRX10; Olfactory receptor OR3-8 | Q8NHB7.2 |
| Olfactory receptor 2C1; OLFmf3; Olfactory receptor 2C2; Olfactory receptor | O95371.3 |
| OR16-1; Olfactory receptor OR16-2 | |
| Olfactory receptor 8B3; Olfactory receptor OR11-311 | Q8NGG8.3 |
| Olfactory receptor 4M2; Olfactory receptor OR15-3 | Q8NGB6.2 |
| Olfactory receptor 2H2; Hs6M1-12; Olfactory receptor 2H3; Olfactory | O95918.2 |
| receptor-like protein FAT11 | |
| Olfactory receptor 52L1; Olfactory receptor OR11-50 | Q8NGH7.4 |
| Olfactory receptor 2A14; OST182; Olfactory receptor 2A6; Olfactory receptor | Q96R47.4 |
| OR7-12 | |
| Olfactory receptor 10C1; Hs6M1-17; Olfactory receptor 10C2 | Q96KK4.3 |
| Olfactory receptor 8S1 | Q8NH09.2 |
| Olfactory receptor 8J1; Olfactory receptor OR11-183 | Q8NGP2.2 |
| Olfactory receptor 6Q1; Olfactory receptor OR11-226 317 aa protein | Q8NGQ2.2 |
| Olfactory receptor 4S2; Olfactory receptor OR11-137 | Q8NH73.2 |
| Olfactory receptor 52N4; Olfactory receptor OR11-64 | Q8N |
| Olfactory receptor 52K1; Olfactory receptor OR11-8 | Q8NGK4.2 |
| Olfactory receptor 52J3; Olfactory receptor OR11-32 | Q8NH60.2 |
| Olfactory receptor 52E2 | Q8NGJ4.2 |
| Olfactory receptor 52A1; HPFH1OR; Odorant receptor HOR3′beta4; Olfactory | Q9UKL2.2 |
| receptor OR11-319 | |
| Olfactory receptor 51V1; Odorant receptor HOR3′beta1; Olfactory receptor | Q9H2C8.2 |
| 51A12; Olfactory receptor OR11-36 | |
| Olfactory receptor 51B5; Odorant receptor HOR5′beta5; Olfactory receptor | Q9H339.2 |
| OR11-37 | |
| Olfactory receptor 10A4; HP2; Olfactory receptor-like protein JCG5 | Q9H209.2 |
| Olfactory receptor 10J1; Olfactory receptor OR1-26; Olfactory receptor-like | P30954.2 |
| protein HGMP07J | |
| Olfactory receptor 4D1; Olfactory receptor 4D3; Olfactory receptor TPCR16 | Q15615.3 |
| Olfactory receptor 12D2; Hs6M1-20; Olfactory receptor OR6-28 | P58182.2 |
| Olfactory receptor 10AC1; Olfactory receptor OR7-5 | Q8NH08.2 |
| Putative olfactory receptor 3A4; Olfactory receptor 17-24; OR17-24; Olfactory | P47883.4 |
| receptor 3A5 | |
| Olfactory receptor 56A4; Olfactory receptor OR11-49 | Q8NGH8.2 |
| Olfactory receptor 52E8; Olfactory receptor OR11-54 | Q6IFG1.3 |
| Olfactory receptor 2A25; Olfactory receptor 2A27 | A4D2G3.2 |
| Olfactory receptor 4K17; Olfactory receptor OR14-29 | Q8NGC6.3 |
| Olfactory receptor 1L1; Olfactory receptor 1L2; Olfactory receptor 9-C; OR9- | Q8NH94.3 |
| C; Olfactory receptor OR9-27 | |
| Olfactory receptor 4A15; Olfactory receptor OR11-118 | Q8NGL6.3 |
| Olfactory receptor 13D1; Olfactory receptor OR9-15 | Q8NGV5.3 |
| Olfactory receptor 8B2; Olfactory receptor OR11-309 | Q96RD0.3 |
| Olfactory receptor 2T1; Olfactory receptor 1-25; OR1-25; Olfactory receptor | O43869.3 |
| OR1-61 | |
| Olfactory receptor 6K3; Olfactory receptor OR1-18 | Q8NGY3.2 |
| Olfactory receptor 4K15; Olfactory receptor OR14-20 | Q8NH41.2 |
| Olfactory receptor 2T4; Olfactory receptor OR1-60 | Q8NH00.2 |
| Olfactory roocptor 1L6; Olfactory receptor 1L7; Olfactory receptor OR9-30 | Q8NGR2.2 |
| Olfactory receptor 13A1; Olfactory receptor OR10-3 | Q8NGR1.2 |
| Olfactory receptor 56B1; Olfactory receptor OR11-65 | Q8N |
| Olfactory receptor 2AK2; Olfactory receptor 2AK1; Olfactory receptor OR1- | Q8NG84.2 |
| 47 335 aa protein | |
| Olfactory receptor 3A3; Olfactory receptor 17-201; OR17-201; Olfactory | P47888.3 |
| receptor 3A6; Olfactory receptor 3A7; Olfactory receptor 3A8; Olfactory | |
| receptor OR17-22 | |
| Olfactory receptor 3A2; Olfactory receptor 17-228; OR17-228; Olfactory | P47893.3 |
| recentor OR17-14 | |
| Olfactory receptor 10R2; Olfactory receptor OR1-8 | Q8NGX6.3 |
| Olfactory receptor 52H1; Olfactory receptor OR11-45 | Q8NGJ2.3 |
| Olfactory receptor 5T2; Olfactory receptor OR11-177 | Q8NGG2.3 |
| Olfactory receptor 6S1; Olfactory receptor OR14-37 | Q8NH40.2 |
| Olfactory receptor 6K6; Olfactory receptor OR1-21 | Q8NGW6.2 |
| Olfactory receptor 5H6; Olfactory receptor OR3-11 | Q8NGV6.2 |
| Olfactory receptor 2D3; Olfactory receptor OR11-89 | Q8NGH3.2 |
| Olfactory receptor 1S2; Olfactory receptor OR11-231 | Q8NGQ3.2 |
| Olfactory receptor 52R1; Olfactory receptor OR11-22 315 aa protein | Q8NGF1.2 |
| Olfactory receptor 51F2; Olfactory receptor OR11-23 | Q8NH61.2 |
| Olfactory receptor 10S1; Olfactory receptor OR11-279 | Q8NGN2.2 |
| Olfactory receptor 52B2; Olfactory receptor OR11-70 | Q96RD2.3 |
| Olfactory receptor 52I2; Olfactory receptor OR11-12 | Q8NH67.3 |
| Olfactory receptor 52B6; Olfactory receptor OR11-47 | Q8NGF0.3 |
| Putative olfactory receptor 52L2; Olfactory receptor OR11-74 | Q8NGH6.3 |
| Olfactory receptor 2C3; Olfactory receptor 2C4; Olfactory receptor 2C5; | Q8N628.3 |
| Olfactory receptor OR1-30 | |
| Olfactory receptor 5T3; Olfactory receptor OR11-178 | Q8NGG3.3 |
| Olfactory receptor 9K2; Olfactory receptor OR12-2 | Q8NGE7.2 |
| Olfactory receptor 7G1; Olfactory receptor 19-15; OR19-15; Olfactory | Q8NGA0.2 |
| receptor OR19-8 | |
| Olfactory receptor 4N4; Olfactory receptor OR15-1; Olfactory receptor | Q8N0Y3.2 |
| OR15-5 | |
| Olfactory receptor 2K2; HTPCRH06; Olfactory receptor OR9-17 | Q8NGT1.2 |
| Olfactory receptor 1S1; Olfactory receptor OR11-232 | Q8NH92.2 |
| Olfactory receptor 1N2; Olfactory receptor OR9-23 | Q8NGR9.2 |
| Olfactory receptor 52K2; Olfactory receptor OR11-7 | Q8NGK3.2 |
| Olfactory receptor 13C3; Olfactory receptor OR9-8 | Q8NGS6.2 |
| Olfactory receptor 4A47; Olfactory receptor OR11-113 309 aa protein | Q6IF82.2 |
| Olfactory receptor 11H1; Olfactory receptor OR22-1 | Q8NG94.3 |
| Olfactory receptor 5H2; Olfactory receptor OR3-10 | Q8NGV7.3 |
| Olfactory receptor 9G4; Olfactory receptor OR11-216 | Q8NGQ1.2 |
| Olfactory receptor 8A1; OST025; Olfactory receptor OR11-318 | Q8NGG7.2 |
| Olfactory receptor 4C13; Olfactory receptor OR11-260 | Q8NGP0.2 |
| Olfactory receptor 1A1; Olfactory receptor 17-7; OR17-7; Olfactory receptor | Q9P1Q5.2 |
| OR17-11 | |
| Olfactory receptor 5AU1; Olfactory receptor OR14-38 | Q8NGC0.2 |
| Olfactory receptor 52N5; Olfactory receptor OR11-62 | Q8NH56.2 |
| Olfactory receptor 11G2; Olfactory receptor OR14-34 | Q8NGC1.2 |
| Olfactory receptor 2D2; HB2; Olfactory receptor 11-610; OR11-610; Olfactory | Q9H210.4 |
| receptor 2D1; Olfactory receptor OR11-88 | |
| Olfactory receptor 51B6; Odorant receptor HOR5′beta6 | Q9H340.2 |
| Olfactory receptor 14K1; Olfactory receptor 5AY1; Olfactory receptor OR1-39 | Q8NGZ2.2 |
| Putative olfactory receptor 9A1; HSHTPRX06 | Q8NGU1.2 |
| Olfactory receptor 14A2; Olfactory receptor 5AX1; Olfactory receptor OR1-31 | Q96R54.2 |
| Olfactory receptor 56A5 | P0C7T3.1 |
| Olfactory receptor 2T7; OST723; olfactory receptor OR1-44 | P0C7T2.1 |
| Putative olfactory receptor 2W5 320 aa protein | A6NFU9.1 |
| Olfactory receptor 52W1; Olfactory receptor OR11-71 | Q6IF63.2 |
| Olfactory receptor 11H12 | B2RN74.1 |
| Olfactory receptor 51JI; Odorant receptor HOR5′beta8; Olfactory receptor | Q9H342.2 |
| 51J2 | |
| Olfactory receptor 9G9 | P0C7N8.1 |
| Olfactory receptor 8U9 | P0C7N5.1 |
| Olfactory receptor 8U8 | P0C7N1.1 |
| Olfactory receptor 11H7; Olfactory receptor OR14-32 | Q8NGC8.2 |
| Olfactory receptor 1P1; Olfactory receptor 17-208; OR17-208; Olfactory | Q8NH06.2 |
| receptor OR17-9 | |
| Olfactory receptor 1E3; Olfactory receptor 17-210; OR17-210; Olfactory | Q8WZA6.2 |
| receptor OR17-7 | |
| Olfactory receptor 8J2 | Q8NGG1.2 |
| Olfactory receptor 5G3; Olfactory receptor 5G6; Olfactory receptor OR11-213 | P0C626.1 |
| Olfactory receptor 4Q2; olfactory receptor OR14-21 | P0C623.1 |
| Olfactory receptor 4E1; Olfactory receptor OR14-43 | P0C645.1 |
| Olfactory receptor 4A8; Olfactory receptor OR11-110 | P0C604.1 |
| Olfactory receptor 5AL1; Olfactory receptor OR11-184 | P0C617.1 |
| Olfactory receptor 5AC1; Olfactory receptor OR3-2 307 aa protein | P0C628.1 |
| Olfactory receptor 52Z1 | P0C646.1 |
| Olfactory receptor 10J4 | P0C629.1 |
| Olfactory receptor 4K3; Olfactory receptor OR14-14 | Q96R72.3 |
| Olfactory receptor 2T6; OST703; Olfactory receptor 2T9 | Q8NHC8.2 |
| Olfactory receptor 1B1; Olfactory receptor 9-B; OR9-B; Olfactory receptor | Q8NGR6.2 |
| OR9-26 | |
| Olfactory receptor 10X1; Olfactory receptor OR1-14 | Q8NGY0.2 |
| Olfactory receptor 51F1 319 aa protein | A6NGY5.1 |
| Olfactory receptor 2V1 | Q8NHB1.2 |
| Olfactory receptor 4C45 | A6NMZ5.1 |
| Olfactory receptor 52A4 | A6NMU1.1 |
| Olfactory receptor 5K4 | A6NMS3.1 |
| Olfactory receptor 2AG2 | A6NM03.1 |
| Olfactory receptor 5H14 | A6NHG9.1 |
| Olfactory receptor 2T8 | A6NH00.1 |
| Olfactory receptor 6C68 | A6NDL8.2 |
| Olfactory receptor 6C6 | A6NF89.1 |
| Olfactory receptor 5K3 | A6NET4.1 |
| Olfactory receptor 5H1; HTPCRX14 | A6NKK0.1 |
| Olfactory receptor 5B21 | A6NL26.1 |
| Olfactory receptor 6C76 | A6NM76.1 |
| Olfactory receptor 6C75 | A6NL08.1 |
| Olfactory receptor 6C74 | A6NCV1.1 |
| Olfactory receptor 6C70 | A6NIJ9.1 |
| Olfactory receptor 6C65 | A6NJZ3.1 |
| Olfactory receptor 5H15 | A6NDH6.1 |
| Olfactory receptor 14I1; Olfactory receptor 5BU1 | A6ND48.1 |
| Olfactory receptor 4C46 | A6NHA9.1 |
| Olfactory receptor 2AT4; Olfactory receptor OR11-265 | A6NND4.1 |
| Olfactory receptor 4F21 | O95013.2 |
| Olfactory receptor 2M5 | A3KFT3.1 |
| Olfactory receptor 2A7; Olfactory receptor OR7-18 | Q96R45.3 |
| Olfactory receptor 3A1; Olfactory receptor 17-40; OR17-40; Olfactory | P47881.2 |
| receptor OR17-15 | |
| Olfactory receptor 2J1; Hs6M1-4; Olfactory receptor 6-5; OR6-5 | Q9GZK6.2 |
| Olfactory receptor 5K2; Olfactory receptor OR3-9 | Q8NHB8.3 |
| Olfactory receptor 4D9; Olfactory receptor OR11-253 | Q8NGE8.3 |
| Olfactory receptor 10A2; HP4; Olfactory receptor OR11-86 | Q9H208.2 |
| Olfactory receptor 7C2; Olfactory receptor 19-18; OR19-18; Olfactory receptor | O60412.4 |
| 7C3; Olfactory receptor OR19-22 | |
| Olfactory receptor 5M3; Olfactory receptor OR11-191 | Q8NGP4.2 |
| Olfactory receptor 10V1; Olfactory receptor OR11-256 | Q8N |
| Olfactory receptor 2A5; Olfactory receptor 2A26; Olfactory receptor 2A8; | Q96R48.2 |
| Olfactory receptor 7-138/7-141; OR7-138; OR7-141 | |
| Olfactory receptor 1Q1; OST226; Olfactory receptor 1Q2; Olfactory receptor | Q15612.3 |
| 1Q3; Olfactory receptor 9-A; 0R9-A; Olfactory receptor OR9-25; Olfactory | |
| receptor TPCR106 | |
| Olfactory receptor 6C3; HSA8 | Q9NZP0.2 |
| Olfactory receptor 6C2; HSA3 | Q9NZP2.2 |
| Olfactory receptor 6C1; OST267 | Q96RD1.2 |
| Olfactory receptor 2T3 | Q8NH03.2 |
| Olfactory receptor 2M2; OST423 | Q96R28.2 |
| Olfactory receptor 5AC2; HSA1 | Q9NZP5.2 |
| Olfactory receptor 6B2; Olfactory receptor OR2-1 | Q6IFH4.2 |
| Olfactory receptor 2A2; Olfactory receptor 2A17; Olfactory receptor OR7-11 | Q6IF42.2 |
| Olfactory receptor 4C16; Olfactory receptor OR11-135 | Q8NGL9.2 |
| Olfactory receptor 2W3; Olfactory receptor 2W8; Olfactory receptor OR1-49 | Q7Z3T1.2 |
| Olfactory receptor 8G1; Olfactory receptor OR11-281; Olfactory receptor | Q15617.2 |
| TPCR25 | |
| Olfactory receptor 52A5; Odorant receptor HOR3′beta5; Olfactory receptor | Q9H2C5.1 |
| OR11-33 | |
| Olfactory receptor 5W2; Olfactory receptor 5W3; Olfactory receptor OR11- | Q8NH69.1 |
| 155 | |
| Olfactory receptor 8U1 | Q8NH10.1 |
| Olfactory receptor 2T10; Olfactory receptor OR1-64 | Q8NGZ9.1 |
| Olfactory receptor 2AJ1 | Q8NGZ0.1 |
| Olfactory receptor 52M1; Olfactory receptor OR11-11 | Q8NGK5.1 |
| Olfactory receptor 9Q2 | Q8NGE9.1 |
| Olfactory receptor 2L3 | Q8NG85.1 |
| Olfactory receptor 10K2; Olfactory receptor OR1-4 | Q6IF99.1 |
| Olfactory receptor 2T2; Olfactory receptor OR1-43 | Q6IF00.1 |
| Olfactory receptor 2T5; Olfactory receptor OR1-62 | Q6IEZ7.1 |
| Olfactory receptor 4F3/4F16/4F29; Olfactory receptor OR1-1 | Q6IEY1.1 |
| Olfactory receptor 4C11; Olfactory receptor OR11-136 | Q6IEV9.1 |
| Olfactory receptor 5M10; Olfactory receptor OR11-207 | Q6IEU7.1 |
| Olfactory receptor 2G6 | Q5TZ20.1 |
| Olfactory receptor 10J3 | Q5JRS4.1 |
| Olfactory receptor 2B11 | Q5JQS5.1 |
| Putative olfactory receptor 2W6; Olfactory receptor OR6-3; Putative olfactory | Q8NHA6.1 |
| receptor 2W7 | |
| Olfactory receptor 10G6; Olfactory receptor OR11-280 | Q8NH81.1 |
| Putative olfactory receptor 10D3; HTPCRX09; Olfactory receptor OR11-293 | Q8NH80.1 |
| Olfactory receptor 11H2; Olfactory receptor OR14-1 | Q8NH07.1 |
| Olfactory receptor 2AP1; Olfactory receptor OR12-9 | Q8NGE2.1 |
| Olfactory receptor 4C5; Olfactory receptor OR11-99 | Q8NGB2.1 |
| Olfactory receptor 7E24; Olfactory receptor OR19-14 | Q6IFN5.1 |
| Olfactory receptor 8G2; Olfactory receptor 8G4; Olfactory receptor OR11-292; | Q6IF36.1 |
| Olfactory receptor TPCR120 | |
| Olfactory receptor 2T27; Olfactory receptor OR1-67 | Q8NH04.1 |
| Olfactory receptor 5T1; Olfactory receptor OR11-179 | Q8NG75.1 |
| Olfactory receptor 4D11 | Q8N |
| Olfactory receptor 4D10; Olfactory receptor OR11-251 | Q8N |
| Olfactory receptor 2T12; Olfactory receptor OR1-57 | Q8NG77.1 |
| Olfactory receptor 51D1; Olfactory receptor OR11-14 | Q8NGF3.1 |
| Olfactory receptor 2T33; Olfactory receptor OR1-56 | Q8NG76.1 |
| Olfactory receptor 1C1; Olfactory receptor OR1-42; Olfactory receptor | Q15619.4 |
| TPCR27 | |
| Olfactory receptor 52B4; Olfactory receptor OR11-3 | Q8NGK2.2 |
| Olfactory receptor 5R1; Olfactory receptor OR11-185 | Q8NH85.1 |
| Olfactory receptor 2V2; Olfactory receptor 2V3; Olfactory receptor OR5-3 | Q96R30.3 |
| Olfactory receptor 2M4; HTPCRX18; OST710; Olfactory receptor OR1-55; | Q96R27.2 |
| Olfactory receptor TPCR100 | |
| Olfactory receptor 2T34; Olfactory receptor OR1-63 | Q8NGX1.1 |
| Olfactory receptor 6A2; Olfactory receptor 11-55; OR11-55; Olfactory | O95222.2 |
| receptor 6A1; Olfactory receptor OR11-83; hP2 olfactory receptor | |
| Olfactory receptor 10W1; Olfactory receptor OR11-236 | Q8NGF6.1 |
| Olfactory receptor 10P1; Olfactory receptor 10P2; Olfactory receptor 10P3; | Q8NGE3.1 |
| Olfactory receptor OR12-7 | |
| Olfactory receptor 14C36; Olfactory receptor 5BF1; Olfactory receptor | Q8NHC7.1 |
| OR1-59 | |
| Olfactory receptor 10AG1; Olfactory receptor OR11-160 | Q8NH19.1 |
| Olfactory receptor 2T11; Olfactory receptor OR1-65 | Q8NH01.1 |
| Olfactory receptor 5M11 | Q96RB7.2 |
| Putative olfactory receptor 1F2; OLFmf2 | Q96R84.2 |
| Olfactory receptor 4F4; HS14a-1-A; Olfactory receptor OR19-3 | Q96R69.2 |
| Olfactory receptor 4C12; Olfactory receptor OR11-259 | Q96R67.2 |
| Olfactory receptor 5B2; OST073; Olfactory receptor OR11-240 | Q96R09.3 |
| Olfactory receptor 51E1; D-GPCR; G-protein coupled receptor 164; Olfactory | Q8TCB6.1 |
| receptor 52A3; Prostate-overexpressed G protein-coupled receptor; Prostate- | |
| specific G protein-coupled receptor 2 | |
| Putative olfactory receptor 14L1; Putative olfactory receptor 5AV1 | Q8NHC6.1 |
| Olfactory receptor 14A16; Olfactory receptor 5AT1; Olfactory receptor | Q8NHC5.1 |
| OR1-45 | |
| Olfactory receptor 10J5; Olfactory receptor OR1-28 | Q8NHC4.1 |
| Olfactory receptor IF12; Hs6M1-35P | Q8NHA8.1 |
| Olfactory receptor 2AE1; Olfactory receptor 2AE2 | Q8NHA4.1 |
| Olfactory receptor 1L3; Olfactory receptor 9-D; OR9-D; Olfactory receptor | Q8NH93.1 |
| OR9-28 | |
| Olfactory receptor 5AK2 | Q8NH90.1 |
| Putative olfactory receptor 5AK3 | Q8NH89.1 |
| Olfactory receptor 9G1; Olfactory receptor 9G5; Olfactory receptor OR11-114 | Q8NH87.1 |
| Olfactory receptor 6X1; Olfactory receptor OR11-270 | Q8NH79.1 |
| Olfactory receptor 56B4; Olfactory receptor OR11-67 | Q8NH76.1 |
| Olfactory receptor 10A6; Olfactory receptor OR11-96 | Q8NH74.1 |
| Olfactory receptor 4C6; Olfactory receptor OR11-138 | Q8NH72.1 |
| Olfactory receptor 4A16; Olfactory receptor OR11-117 | Q8NH70.1 |
| Olfactory receptor 51A7; Olfactory receptor OR11-27 | Q8NH64.1 |
| Olfactory receptor 51H1; Olfactory receptor OR11-25 | Q8NH63.1 |
| Putative olfactory receptor 52P1 | Q8NH57.2 |
| Olfactory receptor 56A3; Olfactory receptor 56A6 | Q8NH54.2 |
| Olfactory receptor 52N1; Olfactory receptor OR11-61 | Q8NH53.1 |
| Olfactory receptor 8K3; Olfactory receptor OR11-181 | Q8NH51.1 |
| Olfactory receptor 8K5; Olfactory receptor OR11-174 | Q8NH50.1 |
| Olfactory receptor 4X1; Olfactory receptor OR11-104 | Q8NH49.1 |
| Olfactory receptor 5B3; Olfactory receptor 5B13; Olfactory receptor OR11- | Q8NH48.1 |
| 239 | |
| Olfactory receptor 4L1; Olfactory receptor 4L2; Olfactory receptor OR14-28 | Q8NH43.1 |
| Olfactory receptor 4K13; Olfactory receptor OR14-27 | Q8NH42.1 |
| Olfactory receptor 4C3; Olfactory receptor OR11-98 | Q8NH37.2 |
| Olfactory receptor 4F5 | Q8NH21.1 |
| Olfactory receptor 4Q3; Olfactory receptor 4Q4; Olfactory receptor OR14-3 | Q8NH05.1 |
| Olfactory receptor 2T29 315 aa protein | Q8NH02.2 |
| Olfactory receptor 6F1; Olfactory receptor OR1-38 | Q8NGZ6.1 |
| Olfactory receptor 2G2; Olfactory receptor OR1-32 | Q8NGZ5.1 |
| Olfactory receptor 2G3; Olfactory receptor OR1-33 309 aa protein | Q8NGZ4.1 |
| Olfactory receptor 13G1; Olfactory receptor OR1-37 | Q8NGZ3.1 |
| Olfactory receptor 2L8; Olfactory receptor OR1-46 | Q8NGY9.1 |
| Putative olfactory receptor 10J6 | Q8NGY7.1 |
| Olfactory receptor 6N2; Olfactory receptor OR1-23 | Q8NGY6.1 |
| Olfactory receptor 6N1 | Q8NGY5.1 |
| Olfactory receptor 6K2; Olfactory receptor OR1-17 | Q8NGY2.1 |
| Olfactory receptor 10Z1; Olfactory receptor OR.1-15 | Q8NGY1.1 |
| Olfactory receptor 6P1; Olfactory receptor OR1-12 | Q8NGX9.1 |
| Olfactory receptor 6Y1; Olfactory receptor 6Y2; Olfactory receptor OR1-11 | Q8NGX8.1 |
| Olfactory receptor 10K1; Olfactory receptor OR1-6 | Q8NGX5.1 |
| Olfactory receptor 10T2; Olfactory receptor OR1-3 | Q8NGX3.1 |
| Olfactory receptor 11L1 | Q8NGX0.1 |
| Olfactory receptor 2Y1; Olfactory receptor OR5-2 | Q8NGV0.1 |
| Putative olfactory receptor 2I1; Putative olfactory receptor 2I2; Putative | Q8NGU4.1 |
| olfactory receptor 2I3; Putative olfactory receptor 2I4 | |
| Olfactory receptor 9A4; Olfactory receptor OR7-1 | Q8NGU2.1 |
| Olfactory receptor 2A1/2A42; Olfactory receptor OR7-16; Olfactory receptor | Q8NGT9.2 |
| OR7-19 | |
| Olfactory receptor 9A2; Olfactory receptor OR7-2 | Q8NGT5.1 |
| Olfactory receptor 13J1; Olfactory receptor OR9-2 | Q8NGT2.1 |
| Olfactory receptor 13C9; Olfactory receptor OR9-13 | Q8NGT0.1 |
| Olfactory receptor 13C2; Olfactory receptor OR9-12 | Q8NGS9.1 |
| Olfactory receptor 13C5; Olfactory receptor OR9-11 | Q8NGS8.1 |
| Olfactory receptor 13C8 | Q8NGS7.1 |
| Olfactory receptor 13F1; Olfactory receptor OR9-6 | Q8NGS4.1 |
| Olfactory receptor 1J1; Olfactory receptor OR9-18 | Q8NGS3.1 |
| Olfactory receptor 1J2; HSA5; HTPCRX15; OST044; Olfactory receptor 1J3; | Q8NGS2.1 |
| Olfactory receptor 1J5; Olfactory receptor OR9-19 | |
| Olfactory receptor 1J4; HTPCRX01; Olfactory receptor OR9-21 | Q8NGS1.1 |
| Olfactory receptor 1N1; Olfactory receptor 1-26; OR1-26; Olfactory receptor | Q8NGS0.1 |
| 1N3; Olfactory receptor OR9-22 | |
| Olfactory receptor 1L8; Olfactory receptor OR9-24 | Q8NGR8.1 |
| Olfactory receptor 1L4; OST046; Olfactory receptor 1L5; Olfactory receptor 9- | Q8NGR5.1 |
| E; OR9-E; Olfactory receptor OR9-29 | |
| Olfactory receptor 5C1; Olfactory receptor 5C2; Olfactory receptor 9-F; OR9-F | Q8NGR4.1 |
| Olfactory receptor 1K1 | Q8NGR3.1 |
| Olfactory receptor 9I1; Olfactory receptor OR11-228 | Q8NGQ6.1 |
| Olfactory receptor 9Q1 | Q8NGQ5.1 |
| Olfactory receptor 10Q1; Olfactory receptor OR11-233 | Q8NGQ4.1 |
| Olfactory receptor 5M1; OST050; Olfactory receptor OR11-208 | Q8NGP8.1 |
| Olfactory receptor 5M8; Olfactory receptor OR11-194 | Q8NGP6.1 |
| Olfactory receptor 5M9; Olfactory receptor OR11-190 | Q8NGP3.1 |
| Putative olfactory receptor 4A4; Olfactory receptor OR11-107 | Q8NGN8.1 |
| Putative olfactory receptor 10D4 | Q8NGN7.1 |
| Olfactory receptor 10G7; Olfactory receptor OR11-283 | Q8NGN6.1 |
| Olfactory receptor 10G8; Olfactory receptor OR11-282 | Q8NGN5.1 |
| Olfactory receptor 10G9; Olfactory receptor 10G10 | Q8NGN4.1 |
| Olfactory receptor 10G4; Olfactory receptor OR11-278 | Q8NGN3.1 |
| Olfactory receptor 6T1; Olfactory receptor OR11-277 | Q8NGN1.1 |
| Olfactory receptor 4D5; Olfactory receptor OR11-276 | Q8NGN0.1 |
| Olfactory receptor 8D4; Olfactory receptor OR11-275 | Q8NGM9.1 |
| Olfactory receptor 6M1; Olfactory receptor OR11-271 | Q8NGM8.1 |
| Olfactory receptor 4C15; Olfactory receptor OR11-127; Olfactory receptor | Q8NGM1.1 |
| OR11-134 | |
| Olfactory receptor 4P4; Olfactory receptor 4P3 | Q8NGL7.1 |
| Olfactory receptor 5D13; Olfactory receptor OR11-142; Olfactory receptor | Q8NGL4.2 |
| OR11-148 | |
| Olfactory receptor 5D14; Olfactory receptor OR11-141; Olfactory receptor | Q8NGL3.1 |
| OR11-150 | |
| Olfactory receptor 5L1; OST262; Olfactory receptor OR11-151 | Q8NGL2.1 |
| Olfactory receptor 5D18; Olfactory receptor OR11-143; Olfactory receptor | Q8NGL1.1 |
| OR11-152 | |
| Olfactory receptor 5L2; HTPCRX16; Olfactory receptor OR11-153 | Q8NGL0.1 |
| Olfactory receptor 5D16; Olfactory receptor OR11-154 | Q8NGK9.1 |
| Olfactory receptor 5211; Olfactory receptor OR11-13 | Q8NGK6.2 |
| Olfactory receptor 51G1; Olfactory receptor 51G3; Olfactory receptor OR11- | Q8NGK1.1 |
| 29 | |
| Olfactory receptor 51G2; Olfactory receptor OR11-28 | Q8NGK0.1 |
| Olfactory receptor 51T1; Olfactory receptor OR11-26 | Q8NGJ9.1 |
| Olfactory receptor 51S1; Olfactory receptor OR11-24 | Q8NGJ8.1 |
| Olfactory receptor 51A2 | Q8NGJ7.1 |
| Olfactory receptor 51A4 | Q8NGJ6.1 |
| Olfactory receptor 51LI; Olfactory receptor OR11-31 | Q8NGJ5.1 |
| Olfactory receptor 52E1 | Q8NGJ3.1 |
| Olfactory receptor 4D6; Olfactory receptor OR11-250 | Q8NGJ1.1 |
| Olfactory receptor 5A1; OST181; Olfactory receptor OR11-249 | Q8NGJ0.1 |
| Olfactory receptor 5AN1; Olfactory receptor OR11-244 | Q8N |
| Putative olfactory receptor 56B2 | Q8N |
| Olfactory receptor 52N2; Olfactory receptor OR11-57 | Q8N |
| Olfactory receptor 52E4; Olfactory receptor OR11-55 | Q8NGH9.1 |
| Olfactory receptor 8B12; Olfactory receptor OR11-317 | Q8NGG6.1 |
| Olfactory receptor 8K1; Olfactory receptor OR11-182 | Q8NGG5.1 |
| Olfactory receptor 8H1; Olfactory receptor OR11-180 | Q8NGG4.1 |
| Olfactory receptor 8J3; Olfactory receptor OR11-173 | Q8NGG0.1 |
| Olfactory receptor 4X2; Olfactory receptor OR11-105 | Q8NGF9.1 |
| Olfactory receptor 4B1; OST208; Olfactory receptor OR11-106 | Q8NGF8.1 |
| Olfactory receptor 5B17; Olfactory receptor 5B20; Olfactory receptor OR11- | Q8NGF7.1 |
| 237 | |
| Olfactory receptor 10A7; Olfactory receptor OR12-6 | Q8NGE5.1 |
| Olfactory receptor 4K14; Olfactory receptor OR14-22 | Q8NGD5.1 |
| Olfactory receptor 4K1; Olfactory receptor OR14-19 | Q8NGD4.1 |
| Olfactory receptor 4K5; Olfactory receptor OR14-16 | Q8NGD3.1 |
| Olfactory receptor 4K2; Olfactory receptor OR14-15 | Q8NGD2.1 |
| Olfactory receptor 4N2; Olfactory receptor OR14-13; Olfactory receptor | Q8NGD1.1 |
| OR14-8 | |
| Olfactory receptor 4M1; Olfactory receptor OR14-7 | Q8NGD0.1 |
| Olfactory receptor 11H4; Olfactory receptor OR14-36 | Q8NGC9.1 |
| Olfactory receptor 11H6; Olfactory receptor OR14-35 | Q8NGC7.1 |
| Olfactory receptor 6J1; Olfactory receptor 6J2 | Q8NGC5.1 |
| Olfactory receptor 10G3; Olfactory receptor OR14-40 | Q8NGC4.1 |
| Olfactory receptor 10G2 | Q8NGC3.1 |
| Olfactory receptor 4E2; Olfactory receptor OR14-42 | Q8NGC2.1 |
| Olfactory receptor 4F6; Olfactory receptor 4F12; Olfactory receptor OR15-15 | Q8NGB9.1 |
| Olfactory receptor 4F15; Olfactory receptor OR15-14 | Q8NGB8.1 |
| Olfactory receptor 4S1; Olfactory receptor OR11-100 | Q8NGB4.1 |
| Olfactory receptor 4F17; Olfactory receptor 4F11; Olfactory receptor 4F18; | Q8NGA8.1 |
| Olfactory receptor 4F19 | |
| Olfactory receptor 10H5; Olfactory receptor OR19-25; Olfactory receptor | Q8NGA6.1 |
| OR19-26 | |
| Olfactory receptor 10H4; Olfactory receptor OR19-28 | Q8NGA5.1 |
| Putative olfactory receptor 7A2; Putative olfactory receptor 7A7 | Q8NGA2.1 |
| Olfactory receptor 1M1; Olfactory receptor 19-6; OR19-6; Olfactory receptor | Q8NGA1.1 |
| OR19-5 | |
| Olfactory receptor 7G2; OST260; Olfactory receptor 19-13; OR19-13; | Q8NG99.1 |
| Olfactory receptor OR19-6 | |
| Olfactory receptor 2Z1; Olfactory receptor 2Z2; Olfactory receptor OR19-4 | Q8NG97.1 |
| Olfactory receptor 7G3; OST085; Olfactory receptor OR19-9 | Q8NG95.1 |
| Olfactory receptor 13H1; Olfactory receptor ORX-1 | Q8NG92.1 |
| Olfactory receptor 8H2; Olfactory receptor OR11-171 | Q8N162.1 |
| Olfactory receptor 6V1; Olfactory receptor OR7-3 | Q8N148.1 |
| Olfactory receptor 8H3; Olfactory receptor OR11-172 | Q8N146.1 |
| Olfactory receptor 5AS1; Olfactory receptor OR11-168 | Q8N127.1 |
| Olfactory receptor 8I2; Olfactory receptor OR11-170 | Q8N0Y5.1 |
| Putative olfactory receptor 2B8; Hs6M1-29P | P59922.1 |
| Olfactory receptor 5J2; Olfactory receptor OR11-266 | Q8NH18.1 |
| Olfactory receptor 2A12; Olfactory receptor OR7-10 | Q8NGT7.1 |
| Olfactory receptor 2M7; Olfactory receptor OR1-58 | Q8NG81.1 |
| Olfactory receptor 2L5; Olfactory receptor 2L11; Olfactory receptor OR1-53 | Q8NG80.1 |
| Olfactory receptor 2L13; Olfactory receptor 2L14 | Q8N349.1 |
| Olfactory receptor 51Q1 | Q8NH59.2 |
| Olfactory receptor 2L2; HTPCRH07; Olfactory receptor 2L12; Olfactory | Q8NH16.1 |
| receptor 2L4 | |
| Olfactory receptor 2T35; Olfactory receptor OR1-66 | Q8NGX2.1 |
| Olfactory receptor 6B3; Olfactory receptor OR2-2 | Q8NGW1.1 |
| Olfactory receptor 6C4; Olfactory receptor OR12-10 | Q8NGE1.1 |
| Olfactory receptor 10AD1; Olfactory receptor OR12-1 | Q8NGE0.1 |
| Olfactory receptor 2M3; Olfactory receptor 2M6; Olfactory receptor OR1-54 | Q8NG83.1 |
| Olfactory receptor 1D4; Olfactory receptor 17-30; OR17-30 | P47884.3 |
| Olfactory receptor 7D2; HTPCRH03; Olfactory receptor 19-4; OR19-4; | Q96RA2.2 |
| Olfactory receptor OR19-10 | |
| Olfactory receptor 13C4; Olfactory receptor OR9-7 | Q8NGS5.1 |
| Olfactory receptor 5AR1; Olfactory receptor OR11-209 | Q8NGP9.1 |
| Olfactory receptor 5A2; Olfactory receptor OR11-248 | Q8N |
| Olfactory receptor 5AP2 | Q8NGF4.1 |
| Olfactory receptor 4N5; Olfactory receptor OR14-33 | Q8IXE1.1 |
| Olfactory receptor 52E6; Olfactory receptor OR11-58 | Q96RD3.2 |
| Olfactory receptor 8B4; Olfactory receptor OR11-315 | Q96RC9.2 |
| Olfactory receptor 5B12; Olfactory receptor 5B16; Olfactory receptor OR11- | Q96R08.2 |
| 241 | |
| Olfactory receptor 5P3; Olfactory receptor OR11-94; Olfactory receptor-like | Q8WZ94.1 |
| protein JCG1 | |
| Olfactory receptor 5P2; Olfactory receptor-like protein JCG3 | Q8WZ92.1 |
| Olfactory receptor 8D1; OST004; Olfactory receptor 8D3; Olfactory receptor | Q8WZ84.1 |
| OR11-301; Olfactory receptor-like protein JCG9 | |
| Olfactory receptor 52D1; Odorant receptor HOR5′beta14; Olfactory receptor | Q9H346.1 |
| OR11-43 | |
| Olfactory receptor 51I2; Odorant receptor HOR5′beta12; Olfactory receptor | Q9H344.1 |
| OR11-38 | |
| Olfactory receptor 51I1; Odorant receptor HOR5′beta11; Olfactory receptor | Q9H343.1 |
| OR11-39 | |
| Olfactory receptor 10H1; Olfactory receptor OR19-27 | Q9Y4A9.1 |
| Olfactory receptor 2W1; Hs6M1-15; Olfactory receptor OR6-13 | Q9Y3N9.1 |
| Olfactory receptor 14J1; Hs6M1-28; Olfactory receptor 5U1; Olfactory | Q9UGF5.1 |
| receptor OR6-25 | |
| Olfactory receptor 2S2; Olfactory receptor OR9-3 | Q9NQN1.2 |
| Olfactory receptor 10A5; HP3; Olfactory receptor 10A1; Olfactory receptor | Q9H207.1 |
| 11-403; OR11-403; Olfactory receptor-like protein JCG6 | |
| Olfactory receptor 2AG1; HT3; Olfactory receptor 2AG3; Olfactory receptor | Q9H205.2 |
| OR11-79 | |
| Olfactory receptor 8D2; Olfactory receptor OR11-303; Olfactory receptor-like | Q9GZM6.1 |
| protein JCG2 | |
| Olfactory receptor 2B2; Hs6M1-10; Olfactory receptor 2B9; Olfactory receptor | Q9GZK3.1 |
| 6-1; OR6-1 | |
| Olfactory receptor 7A5; Olfactory receptor OR19-17; Olfactory receptor | Q15622.2 |
| TPCR92 | |
| Olfactory receptor 8B8; Olfactory receptor TPCR85; Olfactory-like receptor | Q15620.2 |
| JCG8 | |
| Olfactory receptor 10A3; HTPCRX12; Olfactory receptor OR11-97 | P58181.1 |
| Olfactory receptor 4D2; B-lymphocyte membrane protein BC2009; Olfactory | P58180.1 |
| receptor OR17-24 | |
| Olfactory receptor 2B6; Hs6M1-32; Olfactory receptor 2B1; Olfactory receptor | P58173.1 |
| 2B5; Olfactory receptor 5-40; OR5-40; Olfactory receptor 6-31; OR6-31; | |
| Olfactory receptor OR6-4 | |
| Olfactory receptor 1D5; Olfactory receptor 17-31; OR17-31 | P58170.1 |
| Olfactory receptor 5F1; Olfactory receptor 11-10; OR11-10; Olfactory receptor | O95221.2 |
| OR11-167 | |
| Olfactory receptor 2A4; Olfactory receptor 2A10; Olfactory receptor OR6-37 | O95047.1 |
| Olfactory receptor 6B1; Olfactory receptor 7-3; OR7-3; Olfactory receptor | O95007.1 |
| OR7-9 | |
| Olfactory receptor 2F2; Olfactory receptor 7-1; OR7-1; Olfactory receptor | O95006.1 |
| OR7-6 | |
| Olfactory receptor 7A10; OST027; Olfactory receptor OR19-18 | O76100.1 |
| Olfactory receptor 2J2; Hs6M1-6; Olfactory receptor 6-8; OR6-8; Olfactory | O76002.1 |
| receptor OR6-19 | |
| Putative olfactory receptor 2B3; Hs6M1-1; Olfactory receptor OR6-14; OR6-4; | O76000.1 |
| Olfactory receptor 6-4 | |
| Olfactory receptor 1I1; Olfactory receptor 19-20; OR19-20 | O60431.1 |
| Olfactory receptor 10H3; Olfactory receptor OR19-24 | O60404.1 |
| Olfactory receptor 10H2; Olfactory receptor OR19-23 | O60403.1 |
| Olfactory receptor 7A17 | O14581.1 |
| Olfactory receptor 2F1; Olfactory receptor 2F3; Olfactory receptor 2F4; | Q13607.2 |
| Olfactory receptor 2F5; Olfactory receptor-like protein OLF3 | |
| Olfactory receptor 1G1; Olfactory receptor 17-209; OR17-209; Olfactory | P47890.2 |
| receptor 1G2; Olfactory receptor OR17-8 | |
| Olfactory receptor 1E2; Olfactory receptor 17-93/17-135/17-136; OR17-135; | P47887.2 |
| OR17-136; OR17-93; Olfactory receptor 1E4 | |
| Olfactory receptor 1A2; Olfactory receptor 17-6; OR17-6; Olfactory receptor | Q9Y585.1 |
| OR17-10 | |
| Olfactory receptor 7C1; Olfactory receptor 7C4; Olfactory receptor OR19-16; | O76099.1 |
| Olfactory receptor TPCR86 | |
| Olfactory receptor 1F1; Olfactory receptor 16-35; OR16-35; Olfactory receptor | O43749.1 |
| 1F10; Olfactory receptor 1F4; Olfactory receptor 1F5; Olfactory receptor 1F6; | |
| Olfactory receptor 1F7; Olfactory receptor 1F8; Olfactory receptor 1F9; | |
| Olfactory receptor OR16-4 | |
| Olfactory receptor 5I1; Olfactory receptor OR11-159; Olfactory receptor-like | Q13606.1 |
| protein OLF1 | |
| Olfactory receptor 1E1; Olfactory receptor 13-66; OR13-66; Olfactory receptor | P30953.1 |
| 17-2/17-32; OR17-2; OR17-32; Olfactory receptor 1E5; Olfactory receptor | |
| 1E6; Olfactory receptor 5-85; OR5-85; Olfactory receptor OR17-18; Olfactory | |
| receptor-like protein HGMP07I | |
| Olfactory receptor 56A1; Olfactory receptor OR11-75 | Q8NGH5.3 |
| putative odorant receptor 71a [Talaromyces mameffei PM1] | KFX53697.1 |
| hypothetical protein XK86_18365 [Hafnia alvei] | KKI42162.1 |
| hypothetical protein PAST3_12155 [Propionibacterium acnes HL201PA1] | KFC15621.1 |
| hypothetical protein Odosp 2381 [Odoribacter splanchnicus DSM 20712] | ADY33373.1 |
| hypothetical protein LLB_1684 [Le Glonella longbeachae D-4968] | EEZ96489.1 |
| hypothetical protein cypCar_00040615 [Cyprinus carpio] | KTG44310.1 |
| hypothetical protein cypCar_00022850 [Cyprinus carpio] | KTF94953.1 |
| hypothetical protein cypCar_00047049 [Cyprinus carpio] | KTF88600.1 |
| hypothetical protein cypCar_00047378 [Cyprinus carpio] | KTF77827.1 |
| hypothetical protein cypCar_00040594 [Cyprinus carpio] | KTF73152.1 |

Table 4, as shown below, provides a set of odorant receptors.

TABLE 4

| Gene Name | Accession Number |
|---|---|
| odorant receptor [Ostrinia nubilalis] 333 aa protein | BAJ61939.1 GI: 319918821 |
| odorant receptor, partial [Ostrinia nubilalis] 419 aa protein | BAJ61937.1 GI: 319918818 |
| odorant receptor, partial [Ostrinia nubilalis] 314 aa protein | BAJ61935.1 GI: 319918814 |
| odorant receptor [Ostrinia nubilalis] 422 aa protein | BAJ61934.1 GI: 319918812 |
| odorant receptor [Ostrinia nubilalis] 408 aa protein | BAJ61933.1 GI: 319918810 |
| odorant receptor [Ostrinia nubilalis] 424 aa protein | BAJ61932.1 GI: 319918808 |
| odorant receptor [Ostrinia nubilalis] 424 aa protein | BAJ61929.1 GI: 319918797 |
| odorant receptor [Ostrinia nubilalis] 425 aa protein | BAJ61928.1 GI: 319918796 |
| odorant receptor, partial [Ostrinia nubilalis] 89 aa protein | BAJ61938.1 GI: 319918819 |
| odorant receptor, partial [Ostrinia nubilalis] 136 aa protein | BAJ61936.1 GI: 319918816 |
| odorant receptor, partial [Ostrinia nubilalis × Ostrinia scapulalis] | BAJ61931.1 GI: 319918803 |
| 200 aa protein | |
| odorant receptor, partial [Ostrinia nubilalis × Ostrinia scapulalis] | BAJ61930.1 GI: 319918801 |
| 200 aa protein | |
| odorant receptor, partial [Ostrinia palustralis] 383 aa protein | BAI66637.3 GI: 310688057 |
| odorant receptor, partial [Ostrinia nubilalis] 380 aa protein | BAI66625.3 GI: 310688051 |
| odorant receptor, partial [Ostrinia zaguliaevi] 412 aa protein | BAJ22892.1 GI: 308522556 |
| odorant receptor, partial [Ostrinia fumacalis] 406 aa protein | BAJ22891.1 GI: 308522554 |
| odorant receptor, partial [Ostrinia scapulalis] 396 aa protein | BAJ22890.1 GI: 308522552 |
| odorant receptor, partial [Ostrinia scapulalis] 406 aa protein | BAJ22889.1 GI: 308522550 |
| odorant receptor, partial [Ostrinia zealis] 408 aa protein | BAI66649.1 GI: 284010028 |
| odorant receptor, partial [Ostrinia zealis] 397 aa protein | BAI66648.1 GI: 284010026 |
| odorant receptor, partial [Ostrinia zealis] 409 aa protein | BAI66647.1 GI: 284010024 |
| odorant receptor, partial [Ostrinia zealis] 407 aa protein | BAI66646.1 GI: 284010022 |
| odorant receptor, partial [Ostrinia zealis] 409 aa protein | BAI66645.1 GI: 284010020 |
| odorant receptor, partial [Ostrinia zealis] 409 aa protein | BAI66644.1 GI: 284010018 |
| odorant receptor, partial [Ostrinia zaguliaevi] 397 aa protein | BAI66642.1 GI: 284010014 |
| odorant receptor, partial [Ostrinia zaguliaevi] 409 aa protein | BAI66640.1 GI: 284010010 |
| odorant receptor, partial [Ostrinia zaguliaevi] 406 aa protein | BAI66639.1 GI: 284010008 |
| odorant receptor, partial [Ostrinia zaguliaevi] 409 aa protein | BAI66638.1 GI: 284010006 |
| odorant receptor, partial [Ostrinia palustralis] 397 aa protein | BAI66636.1 GI: 284010002 |
| odorant receptor, partial [Ostrinia palustralis] 407 aa protein | BAI66635.1 GI: 284010000 |
| odorant receptor, partial [Ostrinia palustralis] 409 aa protein | BAI66634.1 GI: 284009998 |
| odorant receptor, partial [Ostrinia ovalipennis] 408 aa protein | BAI66633.1 GI: 284009996 |
| odorant receptor, partial [Ostrinia ovalipennis] 323 aa protein | BAI66632.1 GI: 284009994 |
| odorant receptor, partial [Ostrinia ovalipennis] 366 aa protein | BAI66631.1 GI: 284009992 |
| odorant receptor, partial [Ostrinia ovalipennis] 406 aa protein | BAI66630.1 GI: 284009990 |
| odorant receptor, partial [Ostrinia nubilalis] 397 aa protein | BAI66627.1 GI: 284009984 |
| odorant receptor, partial [Ostrinia nubilalis] 416 aa protein | BAI66626.1 GI: 284009982 |
| odorant receptor, partial [Ostrinia nubilalis] 407 aa protein | BAI66624.1 GI: 284009978 |
| odorant receptor, partial [Ostrinia nubilalis] 409 aa protein | BAI66623.1 GI: 284009976 |
| odorant receptor, partial [Ostrinia latipennis] 323 aa protein | BAI66621.1 GI: 284009972 |
| odorant receptor, partial [Ostrinia latipennis] 350 aa protein | BAI66620.1 GI: 284009970 |
| odorant receptor, partial [Ostrinia latipennis] 366 aa protein | BAI66619.1 GI: 284009968 |
| odorant receptor, partial [Ostrinia latipennis] 407 aa protein | BAI66618.1 GI: 284009966 |
| odorant receptor, partial [Ostrinia fumacalis] 408 aa protein | BAI66616.1 GI: 284009962 |
| odorant receptor, partial [Ostrinia fumacalis] 396 aa protein | BAI66615.1 GI: 284009960 |
| odorant receptor, partial [Ostrinia fumacalis] 408 aa protein | BAI66614.1 GI: 284009958 |
| odorant receptor, partial [Ostrinia fumacalis] 408 aa protein | BAI66613.1 GI: 284009956 |
| odorant receptor, partial [Ostrinia fumacalis] 407 aa protein | BAI66612.1 GI: 284009954 |
| odorant receptor, partial [Ostrinia fumacalis] 409 aa protein | BAI66611.1 GI: 284009952 |
| odorant receptor [Ostrinia scapulalis] 422 aa protein | BAI66610.1 GI: 284009950 |
| odorant receptor [Ostrinia scapulalis] 408 aa protein | BAI66609.1 GI: 284009948 |
| odorant receptor [Ostrinia scapulalis] 424 aa protein | BAI66608.1 GI: 284009946 |
| odorant receptor [Ostrinia scapulalis] 433 aa protein | BAI66607.1 GI: 284009944 |
| odorant receptor [Ostrinia scapulalis] 422 aa protein | BAI66605.1 GI: 284009940 |
| odorant receptor [Ostrinia scapulalis] 425 aa protein | BAI66604.1 GI: 284009938 |
| odorant receptor, partial [Ostrinia ovalipennis] 304 aa protein | BAI66629.3 GI: 310688055 |
| odorant receptor, partial [Ostrinia nubilalis] 275 aa protein | BAI66628.2 GI: 310688053 |
| odorant receptor, partial [Ostrinia zaguliaevi] 291 aa protein | BAI66643.1 GI: 284010016 |
| odorant receptor, partial [Ostrinia latipennis] 291 aa protein | BAI66622.1 GI: 284009974 |
| odorant receptor, partial [Ostrinia latipennis] 318 aa protein | BAI66617.1 GI: 284009964 |
| Odorant receptor coreceptor; AgOr7; Gustatory and odorant | Q7QCC7.3 GI: 158563992 |
| receptor 7 478 aa protein | |
| Odorant receptor coreceptor; Gustatory and odorant receptor 7 478 | Q178U6.1 GI: 122117922 |
| aa protein | |
| Putative odorant receptor 19b 387 aa protein | Q8IRZ5.1 GI: 55584079 |
| Putative odorant receptor 69a, isoform A 393 aa protein | Q9VU27.2 GI: 41393542 |
| Putative odorant receptor 69a, isoform B 393 aa protein | P82985.1 GI: 14285634 |
| Putative odorant receptor 65c 410 aa protein | P82984.2 GI: 108935862 |
| Putative odorant receptor 65b 406 aa protein | P82983.2 GI: 108935861 |
| Putative odorant receptor 98b 384 aa protein | Q9VAW0.3 GI: 92090622 |
| Putative odorant receptor 85e 467 aa protein | P81924.3 GI: 54041947 |
| Putative odorant receptor 71a 378 aa protein | Q9VUK5.4 GI: 50403809 |
| Putative odorant receptor 92a 408 aa protein | Q9VDM1.3 GI: 33860192 |
| Putative odorant receptor 83c 397 aa protein | Q9VNK9.2 GI: 14285641 |
| Putative odorant receptor 59c 411 aa protein | Q9W1P7.1 GI: 11387002 |
| Putative odorant receptor 85d 412 aa protein | Q9VHQ2.1 GI: 11386992 |
| Putative odorant receptor 13a [Cerapachys biroi] 194 aa protein | EZA49383.1 GI: 607354771 |
| odorant receptor 300 [Nasonia vitripennis] 395 aa protein | NP_001177714.1 GI: 299782530 |
| odorant receptor 299 [Nasonia vitripennis] 417 aa protein | NP_001177713.1 GI: 299782528 |
| odorant receptor 298 [Nasonia vitripennis] 403 aa protein | NP_001177712.1 GI: 299782526 |
| odorant receptor 289 [Nasonia vitripennis] 398 aa protein | NP_001177710.1 GI: 299782524 |
| odorant receptor 258 [Nasonia vitripennis] 383 aa protein | NP_001177709.1 GI: 299782520 |
| odorant receptor 241 [Nasonia vitripennis] 384 aa protein | NP_001177708.1 GI: 299782517 |
| odorant receptor 216 [Nasonia vitripennis] 406 aa protein | NP_001177707.1 GI: 299782515 |
| odorant receptor 198 [Nasonia vitripennis] 393 aa protein | NP_001177706.1 GI: 299782513 |
| odorant receptor 190 [Nasonia vitripennis] 396 aa protein | NP_001177705.1 GI: 299782511 |
| odorant receptor 160 [Nasonia vitripennis] 402 aa protein | NP_001177703.1 GI: 299782507 |
| odorant receptor 156 [Nasonia vitripennis] 400 aa protein | NP_001177702.1 GI: 299782503 |
| odorant receptor 149 [Nasonia vitripennis] 389 aa protein | NP_001177700.1 GI: 299782500 |
| odorant receptor 96 [Nasonia vitripennis] 385 aa protein | NP_001177699.1 GI: 299782498 |
| odorant receptor 47 [Nasonia vitripennis] 380 aa protein | NP_001177494.1 GI: 299523279 |
| odorant receptor 46 [Nasonia vitripennis] 377 aa protein | NP_001177493.1 GI: 299523277 |
| odorant receptor 45 [Nasonia vitripennis] 372 aa protein | NP_001177492.1 GI: 299523275 |
| odorant receptor 44 [Nasonia vitripennis] 373 aa protein | NP_001177491.1 GI: 299523273 |
| odorant receptor 43 [Nasonia vitripennis] 382 aa protein | NP_001177490.1 GI: 299523271 |
| odorant receptor 37 [Nasonia vitripennis] 410 aa protein | NP_001177488.1 GI: 299523269 |
| odorant receptor 28 [Nasonia vitripennis] 402 aa protein | NP_001177483.1 GI: 299523261 |
| odorant receptor 27 [Nasonia vitripennis] 419 aa protein | NP_001177482.1 GI: 299523255 |
| odorant receptor 26 [Nasonia vitripennis] 408 aa protein | NP_001177481.1 GI: 299523251 |
| odorant receptor 25 [Nasonia vitripennis] 405 aa protein | NP_001177480.1 GI: 299523248 |
| odorant receptor 24 [Nasonia vitripennis] 415 aa protein | NP_001177479.1 GI: 299523246 |
| odorant receptor 22 [Nasonia vitripennis] 397 aa protein | NP_001177477.1 GI: 299523244 |
| odorant receptor 23 [Nasonia vitripennis] 420 aa protein | NP_001177478.1 GI: 299523242 |
| odorant receptor 20 [Nasonia vitripennis] 407 aa protein | NP_001177475.1 GI: 299523240 |
| odorant receptor 21 [Nasonia vitripennis] 409 aa protein | NP_001177476.1 GI: 299523238 |
| odorant receptor 19 [Nasonia vitripennis] 405 aa protein | NP_001177474.1 GI: 299523236 |
| odorant receptor 17 [Nasonia vitripennis] 409 aa protein | NP_001177473.1 GI: 299523231 |
| odorant receptor 16 [Nasonia vitripennis] 408 aa protein | NP_001177472.1 GI: 299523229 |
| odorant receptor 15 [Nasonia vitripennis] 401 aa protein | NP_001177471.1 GI: 299523226 |
| odorant receptor 13 [Nasonia vitripennis] 431 aa protein | NP_001177469.1 GI: 299523217 |
| odorant receptor 12 [Nasonia vitripennis] 410 aa protein | NP_001177468.1 GI: 299523215 |
| odorant receptor 10 [Nasonia vitripennis] 406 aa protein | NP_001177467.1 GI: 299523212 |
| odorant receptor 9 [Nasonia vitripennis] 405 aa protein | NP_001177435.1 GI: 299523119 |
| odorant receptor 8 [Nasonia vitripennis] 399 aa protein | NP_001177434.1 GI: 299523116 |
| odorant receptor 7 [Nasonia vitripennis] 408 aa protein | NP_001177433.1 GI: 299523113 |
| odorant receptor 6 [Nasonia vitripennis] 399 aa protein | NP_001177432.1 GI: 299523110 |
| odorant receptor 5 [Nasonia vitripennis] 428 aa protein | NP_001177431.1 GI: 299523107 |
| odorant receptor 3 [Nasonia vitripennis] 435 aa protein | NP_001177430.1 GI: 299523104 |
| odorant receptor 2 [Nasonia vitripennis] 420 aa protein | NP_001177429.1 GI: 299523100 |
| odorant receptor 159 [Nasonia vitripennis] 399 aa protein | NP_001177423.1 GI: 299523072 |
| odorant receptor 292 [Nasonia vitripennis] 403 aa protein | NP_001177621.1 GI: 299522969 |
| odorant receptor 291 [Nasonia vitripennis] 402 aa protein | NP_001177620.1 GI: 299522967 |
| odorant receptor 286 [Nasonia vitripennis] 413 aa protein | NP_001177619.1 GI: 299522965 |
| odorant receptor 285 [Nasonia vitripennis] 411 aa protein | NP_001177618.1 GI: 299522963 |
| odorant receptor 281 [Nasonia vitripennis] 401 aa protein | NP_001177617.1 GI: 299522961 |
| odorant receptor 279 [Nasonia vitripennis] 403 aa protein | NP_001177616.1 GI: 299522959 |
| odorant receptor 278 [Nasonia vitripennis] 403 aa protein | NP_001177615.1 GI: 299522957 |
| odorant receptor 277 [Nasonia vitripennis] 404 aa protein | NP_001177614.1 GI: 299522955 |
| odorant receptor 273 [Nasonia vitripennis] 407 aa protein | NP_001177612.1 GI: 299522950 |
| odorant receptor 272 [Nasonia vitripennis] 400 aa protein | NP_001177611.1 GI: 299522948 |
| odorant receptor 271 [Nasonia vitripennis] 400 aa protein | NP_001177610.1 GI: 299522946 |
| odorant receptor 269 [Nasonia vitripennis] 408 aa protein | NP_001177609.1 GI: 299522944 |
| odorant receptor 268 [Nasonia vitripennis] 407 aa protein | NP_001177608.1 GI: 299522942 |
| odorant receptor 267 [Nasonia vitripennis] 407 aa protein | NP_001177607.1 GI: 299522940 |
| odorant receptor 264 [Nasonia vitripennis] 409 aa protein | NP_001177605.1 GI: 299522936 |
| odorant receptor 260 [Nasonia vitripennis] 384 aa protein | NP_001177603.1 GI: 299522932 |
| odorant receptor 257 [Nasonia vitripennis] 383 aa protein | NP_001177602.1 GI: 299522930 |
| odorant receptor 256 [Nasonia vitripennis] 386 aa protein | NP_001177601.1 GI: 299522928 |
| odorant receptor 255 [Nasonia vitripennis] 385 aa protein | NP_001177600.1 GI: 299522926 |
| odorant receptor 251 [Nasonia vitripennis] 386 aa protein | NP_001177598.1 GI: 299522922 |
| odorant receptor 250 [Nasonia vitripennis] 381 aa protein | NP_001177597.1 GI: 299522920 |
| odorant receptor 248 [Nasonia vitripennis] 384 aa protein | NP_001177596.1 GI: 299522918 |
| odorant receptor 247 [Nasonia vitripennis] 381 aa protein | NP_001177595.1 GI: 299522916 |
| odorant receptor 245 [Nasonia vitripennis] 384 aa protein | NP_001177594.1 GI: 299522914 |
| odorant receptor 236 [Nasonia vitripennis] 419 aa protein | NP_001177592.1 GI: 299522910 |
| odorant receptor 233 [Nasonia vitripennis] 397 aa protein | NP_001177591.1 GI: 299522908 |
| odorant receptor 232 [Nasonia vitripennis] 399 aa protein | NP_001177590.1 GI: 299522906 |
| odorant receptor 230 [Nasonia vitripennis] 394 aa protein | NP_001177589.1 GI: 299522904 |
| odorant receptor 229 [Nasonia vitripennis] 398 aa protein | NP_001177588.1 GI: 299522902 |
| odorant receptor 226 [Nasonia vitripennis] 399 aa protein | NP_001177586.1 GI: 299522900 |
| odorant receptor 227 [Nasonia vitripennis] 400 aa protein | NP_001177587.1 GI: 299522898 |
| odorant receptor 224 [Nasonia vitripennis] 398 aa protein | NP_001177584.1 GI: 299522896 |
| odorant receptor 225 [Nasonia vitripennis] 396 aa protein | NP_001177585.1 GI: 299522894 |
| odorant receptor 222 [Nasonia vitripennis] 396 aa protein | NP_001177583.1 GI: 299522892 |
| odorant receptor 219 [Nasonia vitripennis] 396 aa protein | NP_001177581.1 GI: 299522890 |
| odorant receptor 221 [Nasonia vitripennis] 403 aa protein | NP_001177582.1 GI: 299522888 |
| odorant receptor 218 [Nasonia vitripennis] 399 aa protein | NP_001177580.1 GI: 299522886 |
| odorant receptor 217 [Nasonia vitripennis] 412 aa protein | NP_001177579.1 GI: 299522884 |
| odorant receptor 207 [Nasonia vitripennis] 397 aa protein | NP_001177577.1 GI: 299522882 |
| odorant receptor 204 [Nasonia vitripennis] 390 aa protein | NP_001177576.1 GI: 299522880 |
| odorant receptor 203 [Nasonia vitripennis] 388 aa protein | NP_001177575.1 GI: 299522878 |
| odorant receptor 202 [Nasonia vitripennis] 390 aa protein | NP_001177574.1 GI: 299522876 |
| odorant receptor 201 [Nasonia vitripennis] 390 aa protein | NP_001177573.1 GI: 299522874 |
| odorant receptor 196 [Nasonia vitripennis] 398 aa protein | NP_001177572.1 GI: 299522872 |
| odorant receptor 194 [Nasonia vitripennis] 406 aa protein | NP_001177570.1 GI: 299522868 |
| odorant receptor 193 [Nasonia vitripennis] 398 aa protein | NP_001177569.1 GI: 299522866 |
| odorant receptor 192 [Nasonia vitripennis] 398 aa protein | NP_001177568.1 GI: 299522864 |
| odorant receptor 191 [Nasonia vitripennis] 392 aa protein | NP_001177567.1 GI: 299522860 |
| odorant receptor 187 [Nasonia vitripennis] 417 aa protein | NP_001177564.1 GI: 299522856 |
| odorant receptor 179 [Nasonia vitripennis] 402 aa protein | NP_001177560.1 GI: 299522848 |
| odorant receptor 173 [Nasonia vitripennis] 393 aa protein | NP_001177557.1 GI: 299522840 |
| odorant receptor 170 [Nasonia vitripennis] 414 aa protein | NP_001177556.1 GI: 299522836 |
| odorant receptor 167 [Nasonia vitripennis] 404 aa protein | NP_001177555.1 GI: 299522833 |
| odorant receptor 166 [Nasonia vitripennis] 406 aa protein | NP_001177554.1 GI: 299522831 |
| odorant receptor 162 [Nasonia vitripennis] 409 aa protein | NP_001177553.1 GI: 299522829 |
| odorant receptor 161 [Nasonia vitripennis] 393 aa protein | NP_001177552.1 GI: 299522827 |
| odorant receptor 158 [Nasonia vitripennis] 400 aa protein | NP_001177551.1 GI: 299522825 |
| odorant receptor 157 [Nasonia vitripennis] 395 aa protein | NP_001177550.1 GI: 299522823 |
| odorant receptor 151 [Nasonia vitripennis] 391 aa protein | NP_001177549.1 GI: 299522821 |
| odorant receptor 147 [Nasonia vitripennis] 395 aa protein | NP_001177548.1 GI: 299522819 |
| odorant receptor 146 [Nasonia vitripennis] 402 aa protein | NP_001177547.1 GI: 299522817 |
| odorant receptor 143 [Nasonia vitripennis] 391 aa protein | NP_001177545.1 GI: 299522815 |
| odorant receptor 142 [Nasonia vitripennis] 395 aa protein | NP_001177544.1 GI: 299522813 |
| odorant receptor 139 [Nasonia vitripennis] 388 aa protein | NP_001177542.1 GI: 299522809 |
| odorant receptor 137 [Nasonia vitripennis] 388 aa protein | NP_001177541.1 GI: 299522807 |
| odorant receptor 135 [Nasonia vitripennis] 386 aa protein | NP_001177540.1 GI: 299522805 |
| odorant receptor 133 [Nasonia vitripennis] 397 aa protein | NP_001177539.1 GI: 299522803 |
| odorant receptor 132 [Nasonia vitripennis] 395 aa protein | NP_001177538.1 GI: 299522801 |
| odorant receptor 128 [Nasonia vitripennis] 367 aa protein | NP_001177536.1 GI: 299522797 |
| odorant receptor 125 [Nasonia vitripennis] 370 aa protein | NP_001177534.1 GI: 299522793 |
| odorant receptor 124 [Nasonia vitripennis] 370 aa protein | NP_001177533.1 GI: 299522788 |
| odorant receptor 118 [Nasonia vitripennis] 395 aa protein | NP_001177531.1 GI: 299522781 |
| odorant receptor 117 [Nasonia vitripennis] 385 aa protein | NP_001177530.1 GI: 299522779 |
| odorant receptor 115 [Nasonia vitripennis] 414 aa protein | NP_001177529.1 GI: 299522777 |
| odorant receptor 111 [Nasonia vitripennis] 397 aa protein | NP_001177526.1 GI: 299522773 |
| odorant receptor 110 [Nasonia vitripennis] 397 aa protein | NP_001177525.1 GI: 299522769 |
| odorant receptor 107 [Nasonia vitripennis] 393 aa protein | NP_001177524.1 GI: 299522767 |
| odorant receptor 103 [Nasonia vitripennis] 394 aa protein | NP_001177522.1 GI: 299522763 |
| odorant receptor 102 [Nasonia vitripennis] 397 aa protein | NP_001177521.1 GI: 299522761 |
| odorant receptor 100 [Nasonia vitripennis] 396 aa protein | NP_001177519.1 GI: 299522759 |
| odorant receptor 101 [Nasonia vitripennis] 404 aa protein | NP_001177520.1 GI: 299522757 |
| odorant receptor 99 [Nasonia vitripennis] 397 aa protein | NP_001177518.1 GI: 299522754 |
| odorant receptor 94 [Nasonia vitripennis] 387 aa protein | NP_001177517.1 GI: 299522752 |
| odorant receptor 93 [Nasonia vitripennis] 390 aa protein | NP_001177516.1 GI: 299522748 |
| odorant receptor 89 [Nasonia vitripennis] 396 aa protein | NP_001177515.1 GI: 299522746 |
| odorant receptor 88 [Nasonia vitripennis] 391 aa protein | NP_001177514.1 GI: 299522744 |
| odorant receptor 86 [Nasonia vitripennis] 385 aa protein | NP_001177512.1 GI: 299522742 |
| odorant receptor 87 [Nasonia vitripennis] 387 aa protein | NP_001177513.1 GI: 299522740 |
| odorant receptor 79 [Nasonia vitripennis] 413 aa protein | NP_001177511.1 GI: 299522738 |
| odorant receptor 78 [Nasonia vitripennis] 402 aa protein | NP_001177510.1 GI: 299522736 |
| odorant receptor 69 [Nasonia vitripennis] 382 aa protein | NP_001177509.1 GI: 299522734 |
| odorant receptor 68 [Nasonia vitripennis] 372 aa protein | NP_001177508.1 GI: 299522732 |
| odorant receptor 66 [Nasonia vitripennis] 376 aa protein | NP_001177506.1 GI: 299522730 |
| odorant receptor 67 [Nasonia vitripennis] 379 aa protein | NP_001177507.1 GI: 299522728 |
| odorant receptor 65 [Nasonia vitripennis] 379 aa protein | NP_001177505.1 GI: 299522726 |
| odorant receptor 64 [Nasonia vitripennis] 381 aa protein | NP_001177504.1 GI: 299522724 |
| odorant receptor 62 [Nasonia vitripennis] 381 aa protein | NP_001177503.1 GI: 299522722 |
| odorant receptor 61 [Nasonia vitripennis] 402 aa protein | NP_001177502.1 GI: 299522720 |
| odorant receptor 60 [Nasonia vitripennis] 412 aa protein | NP_001177501.1 GI: 299522718 |
| odorant receptor 59 [Nasonia vitripennis] 389 aa protein | NP_001177500.1 GI: 299522716 |
| odorant receptor 51 [Nasonia vitripennis] 377 aa protein | NP_001177497.1 GI: 299522710 |
| odorant receptor 31 [Nasonia vitripennis] 400 aa protein | NP_001177485.1 GI: 299507620 |
| odorant receptor 283 [Nasonia vitripennis] 408 aa protein | NP_001164423.2 GI: 289666787 |
| odorant receptor 77 [Nasonia vitripennis] 413 aa protein | NP_001164671.1 GI: 283945552 |
| odorant receptor 76 [Nasonia vitripennis] 417 aa protein | NP_001164670.1 GI: 283945550 |
| odorant receptor 263 [Nasonia vitripennis] 384 aa protein | NP_001164420.2 GI: 283945546 |
| odorant receptor 301 [Nasonia vitripennis] 422 aa protein | NP_001164659.1 GI: 283945514 |
| odorant receptor 1 [Nasonia vitripennis] 475 aa protein | NP_001164465.1 GI: 283436213 |
| odorant receptor 253 [Nasonia vitripennis] 383 aa protein | NP_001164463.1 GI: 283436209 |
| odorant receptor 293 [Nasonia vitripennis] 371 aa protein | NP_001164462.1 GI: 283436207 |
| odorant receptor 98 [Nasonia vitripennis] 395 aa protein | NP_001164458.1 GI: 283436197 |
| odorant receptor 265 [Nasonia vitripennis] 408 aa protein | NP_001164457.1 GI: 283436195 |
| odorant receptor 280 [Nasonia vitripennis] 407 aa protein | NP_001164422.1 GI: 283436107 |
| odorant receptor 243 [Nasonia vitripennis] 382 aa protein | NP_001164417.1 GI: 283436101 |
| odorant receptor 249 [Nasonia vitripennis] 388 aa protein | NP_001164419.1 GI: 283436099 |
| odorant receptor 246 [Nasonia vitripennis] 382 aa protein | NP_001164418.1 GI: 283436097 |
| odorant receptor 168 [Nasonia vitripennis] 401 aa protein | NP_001164416.1 GI: 283436095 |
| odorant receptor 163 [Nasonia vitripennis] 409 aa protein | NP_001164411.1 GI: 283135180 |
| odorant receptor 154 [Nasonia vitripennis] 395 aa protein | NP_001164405.1 GI: 283135167 |
| odorant receptor 141 [Nasonia vitripennis] 392 aa protein | NP_001164404.1 GI: 283135164 |
| odorant receptor 105 [Nasonia vitripennis] 396 aa protein | NP_001164401.1 GI: 283135159 |
| odorant receptor 92 [Nasonia vitripennis] 391 aa protein | NP_001164399.1 GI: 283135153 |
| odorant receptor 85 [Nasonia vitripennis] 392 aa protein | NP_001164398.1 GI: 283135151 |
| odorant receptor 80 [Nasonia vitripennis] 386 aa protein | NP_001164396.1 GI: 283135146 |
| odorant receptor 82 [Nasonia vitripennis] 392 aa protein | NP_001164395.1 GI: 283135140 |
| odorant receptor 81 [Nasonia vitripennis] 387 aa protein | NP_001164394.1 GI: 283135138 |
| odorant receptor 2 [Apis mellifera] 477 aa protein | NP_001128415.1 GI: 201023349 |
| odorant receptor 180 [Nasonia vitripennis] 395 aa protein | NP_001177704.1 GI: 299782509 |
| odorant receptor 276 [Nasonia vitripennis] 403 aa protein | NP_001177613.1 GI: 299522952 |
| odorant receptor 182 [Nasonia vitripennis] 411 aa protein | NP_001177562.1 GI: 299522852 |
| odorant receptor 46a, isoform A-like [Diachasma alloeum] 654 aa | XP_015127274.1 GI: 970885192 |
| protein | |
| odorant receptor 130 [Nasonia vitripennis] 395 aa protein | NP_001177640.2 GI: 299782532 |
| odorant receptor 33b-like [Bactrocera oleae] 712 aa protein | XP_014097126.1 GI: 929374155 |
| odorant receptor coreceptor [Ceratitis capitata] 473 aa protein | NP_001266301.1 GI: 525342887 |
| odorant receptor 296 [Nasonia vitripennis] 387 aa protein | NP_001177711.1 GI: 299782522 |
| odorant receptor 153 [Nasonia vitripennis] 389 aa protein | NP_001177701.1 GI: 299782505 |
| odorant receptor 71 [Nasonia vitripennis] 384 aa protein | NP_001177698.1 GI: 299782496 |
| odorant receptor 288 [Nasonia vitripennis] 399 aa protein | NP_001177643.1 GI: 299528645 |
| odorant receptor 58 [Nasonia vitripennis] 393 aa protein | NP_001177639.1 GI: 299528533 |
| odorant receptor 36 [Nasonia vitripennis] 404 aa protein | NP_001177487.1 GI: 299523266 |
| odorant receptor 35 [Nasonia vitripennis] 422 aa protein | NP_001177486.1 GI: 299523264 |
| odorant receptor 14 [Nasonia vitripennis] 405 aa protein | NP_001177470.1 GI: 299523221 |
| odorant receptor 145 [Nasonia vitripennis] 401 aa protein | NP_001177546.1 GI: 299523210 |
| odorant receptor 294 [Nasonia vitripennis] 354 aa protein | NP_001177622.1 GI: 299522971 |
| odorant receptor 266 [Nasonia vitripennis] 407 aa protein | NP_001177606.1 GI: 299522938 |
| odorant receptor 252 [Nasonia vitripennis] 386 aa protein | NP_001177599.1 GI: 299522924 |
| odorant receptor 242 [Nasonia vitripennis] 385 aa protein | NP_001177593.1 GI: 299522912 |
| odorant receptor 195 [Nasonia vitripennis] 400 aa protein | NP_001177571.1 GI: 299522870 |
| odorant receptor 181 [Nasonia vitripennis] 405 aa protein | NP_001177561.1 GI: 299522850 |
| odorant receptor 140 [Nasonia vitripennis] 393 aa protein | NP_001177543.1 GI: 299522811 |
| odorant receptor 129 [Nasonia vitripennis] 395 aa protein | NP_001177537.1 GI: 299522799 |
| odorant receptor 126 [Nasonia vitripennis] 369 aa protein | NP_001177535.1 GI: 299522795 |
| odorant receptor 114 [Nasonia vitripennis] 409 aa protein | NP_001177528.1 GI: 299522775 |
| odorant receptor 113 [Nasonia vitripennis] 393 aa protein | NP_001177527.1 GI: 299522771 |
| odorant receptor 106 [Nasonia vitripennis] 397 aa protein | NP_001177523.1 GI: 299522765 |
| odorant receptor 53 [Nasonia vitripennis] 384 aa protein | NP_001177498.1 GI: 299522712 |
| odorant receptor 262 [Nasonia vitripennis] 385 aa protein | NP_001164460.2 GI: 283945544 |
| odorant receptor 122 [Nasonia vitripennis] 369 aa protein | NP_001164459.1 GI: 283436199 |
| odorant receptor 275 [Nasonia vitripennis] 405 aa protein | NP_001164421.1 GI: 283436105 |
| odorant receptor 41 [Nasonia vitripennis] 397 aa protein | NP_001164391.1 GI: 283135132 |
| odorant receptor 295 [Nasonia vitripennis] 370 aa protein | NP_001177623.1 GI: 299522975 |
| odorant receptor 188 [Nasonia vitripennis] 395 aa protein | NP_001177565.1 GI: 299522858 |
| odorant receptor 175 [Nasonia vitripennis] 393 aa protein | NP_001177558.1 GI: 299522844 |
| odorant receptor 56 [Nasonia vitripennis] 371 aa protein | NP_001177499.1 GI: 299522714 |
| odorant receptor 48 [Nasonia vitripennis] 395 aa protein | NP_001177495.1 GI: 299522708 |
| odorant receptor 38 [Nasonia vitripennis] 414 aa protein | NP_001177489.1 GI: 299528647 |
| odorant receptor 29 [Nasonia vitripennis] 403 aa protein | NP_001177484.1 GI: 299523259 |
| odorant receptor 261 [Nasonia vitripennis] 381 aa protein | NP_001177604.1 GI: 299522934 |
| odorant receptor 189 [Nasonia vitripennis] 395 aa protein | NP_001177566.1 GI: 299522862 |
| odorant receptor 183 [Nasonia vitripennis] 405 aa protein | NP_001177563.1 GI: 299522854 |
| odorant receptor 177 [Nasonia vitripennis] 392 aa protein | NP_001177559.1 GI: 299522846 |
| odorant receptor 119 [Nasonia vitripennis] 391 aa protein | NP_001177532.1 GI: 299522786 |
| odorant receptor 134 [Nasonia vitripennis] 395 aa protein | NP_001177696.1 GI: 299782491 |
| odorant receptor 72 [Nasonia vitripennis] 383 aa protein | NP_001177641.1 GI: 299528641 |
| odorant receptor coreceptor-like [Diuraphis noxia] 167 aa protein | XP_015371514.1 GI: 985412051 |
| odorant receptor 46a, isoform B-like [Diuraphis noxia] 251 aa | XP_015367780.1 GI: 985400241 |
| protein | |
| odorant receptor 46a, isoform B-like [Diuraphis noxia] 129 aa | XP_015367779.1 GI: 985400239 |
| protein | |
| odorant receptor 22c-like [Diuraphis noxia] 176 aa protein | XP_015367764.1 GI: 985400213 |
| odorant receptor 85b-like [Diuraphis noxia] 219 aa protein | XP_015379800.1 GI: 985390295 |
| odorant receptor coreceptor [Diachasma alloeum] 478 aa protein | XP_015126208.1 GI: 970919070 |
| odorant receptor 46a, isoform B-like [Diachasma alloeum] 391 aa | XP_015126084.1 GI: 970918843 |
| protein | |
| putative odorant receptor 65b [Diachasma alloeum] 342 aa protein | XP_015125987.1 GI: 970918672 |
| odorant receptor 13a-like [Diachasma alloeum] 417 aa protein | XP_015125770.1 GI: 970918327 |
| odorant receptor 30a-like [Diachasma alloeum] 160 aa protein | XP_015125618.1 GI: 970918086 |
| odorant receptor 30a-like [Diachasma alloeum] 125 aa protein | XP_015125102.1 GI: 970917269 |
| odorant receptor 30a-like [Diachasma alloeum] 125 aa protein | XP_015125054.1 GI: 970917185 |
| odorant receptor 13a-like [Diachasma alloeum] 395 aa protein | XP_015125011.1 GI: 970917102 |
| odorant receptor 13a-like [Diachasma alloeum] 411 aa protein | XP_015125010.1 GI: 970917100 |
| odorant receptor 13a-like [Diachasma alloeum] 412 aa protein | XP_015125009.1 GI: 970917098 |
| odorant receptor 22c-like [Diachasma alloeum] 417 aa protein | XP_015124997.1 GI: 970917076 |
| odorant receptor 4-like [Diachasma alloeum] 396 aa protein | XP_015124996.1 GI: 970917074 |
| odorant receptor 4-like [Diachasma alloeum] 191 aa protein | XP_015124994.1 GI: 970917070 |
| odorant receptor 22c-like [Diachasma alloeum] 398 aa protein | XP_015124993.1 GI: 970917068 |
| odorant receptor 67c-like isoform X3 [Diachasma alloeum] 350 aa | XP_015123023.1 GI: 970913425 |
| protein | |
| odorant receptor 13a-like isoform X1 [Diachasma alloeum] 386 aa | XP_015123021.1 GI: 970913421 |
| protein | |
| odorant receptor 46a, isoform A-like [Diachasma alloeum] 386 aa | XP_015122891.1 GI: 970913183 |
| protein | |
| odorant receptor Or1-like [Diachasma alloeum] 401 aa protein | XP_015122890.1 GI: 970913181 |
| odorant receptor 13a-like [Diachasma alloeum] 385 aa protein | XP_015122297.1 GI: 970912091 |
| odorant receptor coreceptor-like [Diachasma alloeum] 160 aa | XP_015122295.1 GI: 970912087 |
| protein | |
| odorant receptor 82a-like [Diachasma alloeum] 281 aa protein | XP_015122294.1 GI: 970912085 |
| odorant receptor 13a-like [Diachasma alloeum] 398 aa protein | XP_015122293.1 GI: 970912083 |
| odorant receptor 67c-like [Diachasma alloeum] 225 aa protein | XP_015122289.1 GI: 970912076 |
| odorant receptor 4-like isoform X2 [Diachasma alloeum] 384 aa | XP_015122288.1 GI: 970912074 |
| protein | |
| odorant receptor 4-like isoform X1 [Diachasma alloeum] 390 aa | XP_015122287.1 GI: 970912072 |
| protein | |
| odorant receptor 4-like isoform X1 [Diachasma alloeum] 390 aa | XP_015122286.1 GI: 970912070 |
| protein | |
| odorant receptor 67c-like [Diachasma alloeum] 389 aa protein | XP_015122278.1 GI: 970912056 |
| odorant receptor 4 [Diachasma alloeum] 102 aa protein | XP_015122277.1 GI: 970912054 |
| odorant receptor 82a-like, partial [Diachasma alloeum] 274 aa | XP_015122272.1 GI: 970912046 |
| protein | |
| odorant receptor 4-like [Diachasma alloeum] 378 aa protein | XP_015121583.1 GI: 970910790 |
| odorant receptor 13a-like [Diachasma alloeum] 431 aa protein | XP_015121344.1 GI: 970910338 |
| odorant receptor 13a-like [Diachasma alloeum] 458 aa protein | XP_015121343.1 GI: 970910336 |
| odorant receptor Or2-like [Diachasma alloeum] 413 aa protein | XP_015120978.1 GI: 970909674 |
| odorant receptor 82a-like [Diachasma alloeum] 116 aa protein | XP_015120698.1 GI: 970909157 |
| odorant receptor 13a-like [Diachasma alloeum] 122 aa protein | XP_015120696.1 GI: 970909155 |
| odorant receptor 46a, isoform A-like [Diachasma alloeum] 391 aa | XP_015120217.1 GI: 970908285 |
| protein | |
| odorant receptor Or1-like, partial [Diachasma alloeum] 360 aa | XP_015119834.1 GI: 970907577 |
| protein | |
| odorant receptor Or2-like [Diachasma alloeum] 193 aa protein | XP_015119359.1 GI: 970906702 |
| odorant receptor 67b-like [Diachasma alloeum] 169 aa protein | XP_015118342.1 GI: 970904876 |
| odorant receptor 22c-like [Diachasma alloeum] 431 aa protein | XP_015118336.1 GI: 970904864 |
| putative odorant receptor 71a [Diachasma alloeum] 302 aa protein | XP_015118334.1 GI: 970904862 |
| odorant receptor 2a-like [Diachasma alloeum] 419 aa protein | XP_015117540.1 GI: 970903392 |
| odorant receptor Or2-like [Diachasma alloeum] 202 aa protein | XP_015117539.1 GI: 970903390 |
| odorant receptor 85f-like [Diachasma alloeum] 392 aa protein | XP_015117537.1 GI: 970903386 |
| putative odorant receptor 85d [Diachasma alloeum] 402 aa protein | XP_015117136.1 GI: 970902641 |
| odorant receptor 22c-like [Diachasma alloeum] 402 aa protein | XP_015117110.1 GI: 970902593 |
| odorant receptor 22c-like [Diachasma alloeum] 266 aa protein | XP_015116846.1 GI: 970902114 |
| odorant receptor 49b-like [Diachasma alloeum] 279 aa protein | XP_015116845.1 GI: 970902112 |
| odorant receptor 13a-like isoform X2 [Diachasma alloeum] 321 aa | XP_015115896.1 GI: 970900372 |
| protein | |
| odorant receptor 13a-like isoform X1 [Diachasma alloeum] 325 aa | XP_015115895.1 GI: 970900370 |
| protein | |
| odorant receptor 13a-like [Diachasma alloeum] 386 aa protein | XP_015115894.1 GI: 970900368 |
| odorant receptor 46a, isoform B-like [Diachasma alloeum] 399 aa | XP_015115875.1 GI: 970900334 |
| protein | |
| odorant receptor 33a-like [Diachasma alloeum] 343 aa protein | XP_015115812.1 GI: 970900224 |
| odorant receptor 13a-like [Diachasma alloeum] 380 aa protein | XP_015115810.1 GI: 970900220 |
| odorant receptor Or1-like [Diachasma alloeum] 390 aa protein | XP_015115473.1 GI: 970899618 |
| odorant receptor 49b-like [Diachasma alloeum] 386 aa protein | XP_015114827.1 GI: 970898436 |
| putative odorant receptor 98b [Diachasma alloeum] 367 aa protein | XP_015114784.1 GI: 970898359 |
| odorant receptor 85b [Diachasma alloeum] 393 aa protein | XP_015114365.1 GI: 970897598 |
| odorant receptor 63a-like [Diachasma alloeum] 383 aa protein | XP_015114103.1 GI: 970897122 |
| odorant receptor 67c-like [Diachasma alloeum] 116 aa protein | XP_015114099.1 GI: 970897116 |
| odorant receptor 82a-like [Diachasma alloeum] 388 aa protein | XP_015114079.1 GI: 970897076 |
| odorant receptor Or2-like [Diachasma alloeum] 379 aa protein | XP_015114021.1 GI: 970896976 |
| odorant receptor Or2-like [Diachasma alloeum] 379 aa protein | XP_015114020.1 GI: 970896974 |
| odorant receptor 82a-like [Diachasma alloeum] 325 aa protein | XP_015112771.1 GI: 970894647 |
| putative odorant receptor 92a [Diachasma alloeum] 152 aa protein | XP_015112770.1 GI: 970894645 |
| odorant receptor 13a-like [Diachasma alloeum] 403 aa protein | XP_015112769.1 GI: 970894643 |
| odorant receptor 82a-like [Diachasma alloeum] 403 aa protein | XP_015112590.1 GI: 970894312 |
| odorant receptor 82a-like [Diachasma alloeum] 403 aa protein | XP_015112589.1 GI: 970894310 |
| odorant receptor 4-like [Diachasma alloeum] 409 aa protein | XP_015112587.1 GI: 970894308 |
| odorant receptor 85c-like [Diachasma alloeum] 411 aa protein | XP_015112586.1 GI: 970894306 |
| odorant receptor 82a-like [Diachasma alloeum] 412 aa protein | XP_015112585.1 GI: 970894304 |
| odorant receptor 67c-like [Diachasma alloeum] 411 aa protein | XP_015112584.1 GI: 970894302 |
| odorant receptor Or2-like [Diachasma alloeum] 415 aa protein | XP_015112582.1 GI: 970894300 |
| odorant receptor 13a-like [Diachasma alloeum] 365 aa protein | XP_015111780.1 GI: 970892852 |
| odorant receptor 13a-like [Diachasma alloeum] 407 aa protein | XP_015111777.1 GI: 970892846 |
| odorant receptor 13a-like [Diachasma alloeum] 163 aa protein | XP_015111079.1 GI: 970891543 |
| odorant receptor 42b-like [Diachasma alloeum] 134 aa protein | XP_015111029.1 GI: 970891451 |
| odorant receptor 59b-like [Diachasma alloeum] 420 aa protein | XP_015110864.1 GI: 970891147 |
| odorant receptor 33c-like [Diachasma alloeum] 428 aa protein | XP_015110846.1 GI: 970891111 |
| odorant receptor 47b-like [Diachasma alloeum] 424 aa protein | XP_015110804.1 GI: 970891036 |
| odorant receptor 33b-like [Diachasma alloeum] 415 aa protein | XP_015110803.1 GI: 970891034 |
| odorant receptor coreceptor-like [Diachasma alloeum] 430 aa | XP_015110802.1 GI: 970891032 |
| protein | |
| odorant receptor 33b-like [Diachasma alloeum] 435 aa protein | XP_015110787.1 GI: 970891004 |
| odorant receptor 46a, isoform A-like isoform X2 [Diachasma | XP_015110556.1 GI: 970890577 |
| alloeum] 358 aa protein | |
| putative odorant receptor 85d isoform X1 [Diachasma alloeum] | XP_015110555.1 GI: 970890575 |
| 395 aa protein | |
| putative odorant receptor 92a [Diachasma alloeum] 249 aa protein | XP_015110532.1 GI: 970890535 |
| odorant receptor 13a-like [Diachasma alloeum] 389 aa protein | XP_015110383.1 GI: 970890261 |
| odorant receptor 4-like [Diachasma alloeum] 231 aa protein | XP_015110343.1 GI: 970890187 |
| odorant receptor 10a-like [Diachasma alloeum] 268 aa protein | XP_015109340.1 GI: 970888368 |
| odorant receptor 4-like [Diachasma alloeum] 278 aa protein | XP_015108894.1 GI: 970887560 |
| odorant receptor 10a-like [Diachasma alloeum] 388 aa protein | XP_015108891.1 GI: 970887554 |
| odorant receptor 4-like [Diachasma alloeum] 126 aa protein | XP_015108890.1 GI: 970887552 |
| odorant receptor 67c-like [Diachasma alloeum] 381 aa protein | XP_015108889.1 GI: 970887550 |
| putative odorant receptor 85e [Diachasma alloeum] 422 aa protein | XP_015108502.1 GI: 970886846 |
| odorant receptor Or1-like isoform X3 [Diachasma alloeum] 425 aa | XP_015127537.1 GI: 970885664 |
| protein | |
| odorant receptor Or1-like isoform X2 [Diachasma alloeum] 450 aa | XP_015127536.1 GI: 970885662 |
| protein | |
| odorant receptor Or1-like isoform X1 [Diachasma alloeum] 460 aa | XP_015127535.1 GI: 970885660 |
| protein | |
| odorant receptor Or1-like [Diachasma alloeum] 350 aa protein | XP_015127275.1 GI: 970885194 |
| odorant receptor 46a, isoform A-like [Diachasma alloeum] 391 aa | XP_015127273.1 GI: 970885190 |
| protein | |
| odorant receptor Or2-like [Diachasma alloeum] 416 aa protein | XP_015127033.1 GI: 970884759 |
| odorant receptor 22c-like [Diachasma alloeum] 182 aa protein | XP_015127018.1 GI: 970884735 |
| odorant receptor 49b-like [Diachasma alloeum] 157 aa protein | XP_015127017.1 GI: 970884733 |
| odorant receptor 4-like [Diachasma alloeum] 259 aa protein | XP_015126452.1 GI: 970883704 |
| putative odorant receptor 71a [Diachasma alloeum] 334 aa protein | XP_015126451.1 GI: 970883702 |
| odorant receptor 67a-like [Diachasma alloeum] 316 aa protein | XP_015124669.1 GI: 970883032 |
| odorant receptor 43a-like [Diachasma alloeum] 185 aa protein | XP_015124656.1 GI: 970883030 |
| odorant receptor 4-like [Diachasma alloeum] 388 aa protein | XP_015117493.1 GI: 970881492 |
| odorant receptor 67c-like [Diachasma alloeum] 343 aa protein | XP_015117460.1 GI: 970881486 |
| odorant receptor 13a-like isoform X2 [Diachasma alloeum] 400 aa | XP_015117447.1 GI: 970881484 |
| protein | |
| odorant receptor 13a-like [Diachasma alloeum] 401 aa protein | XP_015117410.1 GI: 970881478 |
| odorant receptor coreceptor-like [Diachasma alloeum] 263 aa | XP_015116354.1 GI: 970881285 |
| protein | |
| odorant receptor 47a-like [Diachasma alloeum] 204 aa protein | XP_015116327.1 GI: 970881281 |
| putative odorant receptor 98b [Diachasma alloeum] 390 aa protein | XP_015116314.1 GI: 970881279 |
| odorant receptor 4-like [Diachasma alloeum] 338 aa protein | XP_015115419.1 GI: 970881109 |
| odorant receptor Or2-like [Diachasma alloeum] 454 aa protein | XP_015112971.1 GI: 970880651 |
| odorant receptor Or2-like [Diachasma alloeum] 385 aa protein | XP_015112944.1 GI: 970880647 |
| odorant receptor 49b-like [Diachasma alloeum] 427 aa protein | XP_015112917.1 GI: 970880643 |
| odorant receptor 49b-like [Diachasma alloeum] 381 aa protein | XP_015112905.1 GI: 970880641 |
| odorant receptor 13a-like [Plutella xylostella] 424 aa protein | NP_001292415.1 GI: 770075498 |
| odorant receptor 24a-like, partial [Halyomorpha halys] 99 aa | XP_014293293.1 GI: 939698236 |
| protein | |
| odorant receptor 59b-like [Halyomorpha halys] 417 aa protein | XP_014293234.1 GI: 939698126 |
| odorant receptor 85b-like [Halyomorpha halys] 399 aa protein | XP_014292083.1 GI: 939695930 |
| odorant receptor 22c-like [Halyomorpha halys] 411 aa protein | XP_014292012.1 GI: 939695795 |
| putative odorant receptor 71a, partial [Halyomorpha halys] 240 aa | XP_014291481.1 GI: 939694804 |
| protein | |
| odorant receptor 24a-like [Halyomorpha halys] 127 aa protein | XP_014290811.1 GI: 939693500 |
| odorant receptor 85b-like [Halyomorpha halys] 83 aa protein | XP_014290807.1 GI: 939693494 |
| odorant receptor 85b-like [Halyomorpha halys] 204 aa protein | XP_014290806.1 GI: 939693492 |
| odorant receptor 22c-like [Halyomorpha halys] 420 aa protein | XP_014290805.1 GI: 939693490 |
| odorant receptor 4-like [Halyomorpha halys] 420 aa protein | XP_014290804.1 GI: 939693488 |
| odorant receptor 4-like [Halyomorpha halys] 208 aa protein | XP_014290613.1 GI: 939693125 |
| odorant receptor 43a-like [Halyomorpha halys] 152 aa protein | XP_014290611.1 GI: 939693123 |
| odorant receptor 4-like [Halyomorpha halys] 366 aa protein | XP_014290317.1 GI: 939692579 |
| odorant receptor 49a-like [Halyomorpha halys] 387 aa protein | XP_014289672.1 GI: 939691321 |
| odorant receptor 24a-like [Halyomorpha halys] 419 aa protein | XP_014289234.1 GI: 939690515 |
| odorant receptor 82a-like [Halyomorpha halys] 391 aa protein | XP_014289202.1 GI: 939690459 |
| odorant receptor 82a-like [Halyomorpha halys] 391 aa protein | XP_014289201.1 GI: 939690457 |
| odorant receptor 82a-like [Halyomorpha halys] 391 aa protein | XP_014289200.1 GI: 939690455 |
| odorant receptor 43a-like [Halyomorpha halys] 319 aa protein | XP_014289020.1 GI: 939690118 |
| odorant receptor 67c-like isoform X3 [Halyomorpha halys] 156 aa | XP_014288707.1 GI: 939689526 |
| protein | |
| odorant receptor 67c-like isoform X2 [Halyomorpha halys] 319 aa | XP_014288705.1 GI: 939689524 |
| protein | |
| odorant receptor 67c-like isoform X1 [Halyomorpha halys] 349 aa | XP_014288704.1 GI: 939689522 |
| protein | |
| odorant receptor 24a [Halyomorpha halys] 414 aa protein | XP_014288393.1 GI: 939688944 |
| odorant receptor 24a [Halyomorpha halys] 414 aa protein | XP_014288391.1 GI: 939688942 |
| odorant receptor 4-like [Halyomorpha halys] 378 aa protein | XP_014287492.1 GI: 939686767 |
| putative odorant receptor 71a [Halyomorpha halys] 140 aa protein | XP_014287487.1 GI: 939686757 |
| odorant receptor 85b-like [Halyomorpha halys] 389 aa protein | XP_014287367.1 GI: 939686386 |
| odorant receptor 43b-like [Halyomorpha halys] 389 aa protein | XP_014287042.1 GI: 939685352 |
| odorant receptor 4-like [Halyomorpha halys] 389 aa protein | XP_014287040.1 GI: 939685346 |
| putative odorant receptor 92a [Halyomorpha halys] 373 aa protein | XP_014286385.1 GI: 939683385 |
| odorant receptor coreceptor-like [Halyomorpha halys] 133 aa | XP_014285169.1 GI: 939679600 |
| protein | |
| odorant receptor 4-like [Halyomorpha halys] 349 aa protein | XP_014282544.1 GI: 939671925 |
| putative odorant receptor 85d isoform X1 [Halyomorpha halys] | XP_014282484.1 GI: 939671750 |
| 270 aa protein | |
| odorant receptor 49a-like isoform X1 [Halyomorpha halys] 175 aa | XP_014282386.1 GI: 939671478 |
| protein | |
| odorant receptor 33a-like [Halyomorpha halys] 406 aa protein | XP_014281821.1 GI: 939670017 |
| odorant receptor coreceptor isoform X2 [Halyomorpha halys] 474 | XP_014279420.1 GI: 939663702 |
| aa protein | |
| odorant receptor coreceptor isoform X1 [Halyomorpha halys] 474 | XP_014279419.1 GI: 939663700 |
| aa protein | |
| odorant receptor 33a-like [Halyomorpha halys] 247 aa protein | XP_014278712.1 GI: 939661914 |
| odorant receptor 67c-like [Halyomorpha halys] 417 aa protein | XP_014278702.1 GI: 939661887 |
| odorant receptor 22c-like [Halyomorpha halys] 426 aa protein | XP_014276985.1 GI: 939657423 |
| odorant receptor 4-like [Halyomorpha halys] 284 aa protein | XP_014276746.1 GI: 939656832 |
| odorant receptor 94a-like [Halyomorpha halys] 339 aa protein | XP_014276741.1 GI: 939656820 |
| odorant receptor 22c-like [Halyomorpha halys] 400 aa protein | XP_014275988.1 GI: 939654773 |
| odorant receptor 24a-like [Halyomorpha halys] 412 aa protein | XP_014275211.1 GI: 939652560 |
| odorant receptor 30a-like [Halyomorpha halys] 389 aa protein | XP_014274900.1 GI: 939651712 |
| odorant receptor 47a-like [Halyomorpha halys] 427 aa protein | XP_014274444.1 GI: 939650476 |
| odorant receptor 67c-like [Halyomorpha halys] 154 aa protein | XP_014272634.1 GI: 939645252 |
| odorant receptor 83a-like [Halyomorpha halys] 416 aa protein | XP_014271039.1 GI: 939640611 |
| odorant receptor 4-like isoform X3 [Halyomorpha halys] 354 aa | XP_014270190.1 GI: 939638174 |
| protein | |
| odorant receptor 4-like isoform X2 [Halyomorpha halys] 376 aa | XP_014270189.1 GI: 939638171 |
| protein | |
| odorant receptor 4-like isoform X1 [Halyomorpha halys] 408 aa | XP_014270188.1 GI: 939638168 |
| protein | |
| odorant receptor 24a-like [Halyomorpha halys] 414 aa protein | XP_014270184.1 GI: 939638154 |
| odorant receptor 24a-like [Halyomorpha halys] 81 aa protein | XP_014294800.1 GI: 939638065 |
| odorant receptor 24a-like [Halyomorpha halys] 134 aa protein | XP_014294799.1 GI: 939638061 |
| odorant receptor 85b-like [Halyomorpha halys] 99 aa protein | XP_014294798.1 GI: 939638059 |
| odorant receptor 24a-like [Halyomorpha halys] 415 aa protein | XP_014294793.1 GI: 939638051 |
| odorant receptor 30a-like [Halyomorpha halys] 415 aa protein | XP_014294791.1 GI: 939638047 |
| odorant receptor 7a-like [Halyomorpha halys] 124 aa protein | XP_014294788.1 GI: 939638038 |
| odorant receptor 24a-like [Halyomorpha halys] 415 aa protein | XP_014294787.1 GI: 939638035 |
| odorant receptor 9a-like [Halyomorpha halys] 382 aa protein | XP_014294442.1 GI: 939637038 |
| odorant receptor Or1-like isoform X1 [Halyomorpha halys] 384 aa | XP_014294439.1 GI: 939637034 |
| protein | |
| odorant receptor 85b-like isoform X1 [Halyomorpha halys] 389 aa | XP_014293859.1 GI: 939635389 |
| protein | |
| odorant receptor 46a, isoform B-like [Halyomorpha halys] 390 aa | XP_014291074.1 GI: 939634209 |
| protein | |
| odorant receptor 49b-like [Halyomorpha halys] 288 aa protein | XP_014281510.1 GI: 939631411 |
| odorant receptor 4-like [Halyomorpha halys] 397 aa protein | XP_014281005.1 GI: 939631291 |
| odorant receptor 85c-like [Halyomorpha halys] 99 aa protein | XP_014280802.1 GI: 939631237 |
| odorant receptor 85b-like [Halyomorpha halys] 99 aa protein | XP_014280790.1 GI: 939631235 |
| odorant receptor 24a-like isoform X1 [Halyomorpha halys] 397 aa | XP_014280768.1 GI: 939631231 |
| protein | |
| odorant receptor 7a-like [Halyomorpha halys] 310 aa protein | XP_014280756.1 GI: 939631229 |
| odorant receptor 4-like [Halyomorpha halys] 413 aa protein | XP_014280744.1 GI: 939631227 |
| odorant receptor 82a-like [Halyomorpha halys] 411 aa protein | XP_014280723.1 GI: 939631221 |
| odorant receptor 82a [Halyomorpha halys] 419 aa protein | XP_014280696.1 GI: 939631215 |
| odorant receptor 24a-like [Halyomorpha halys] 417 aa protein | XP_014280623.1 GI: 939631200 |
| odorant receptor 24a-like [Halyomorpha halys] 431 aa protein | XP_014280612.1 GI: 939631197 |
| odorant receptor 49b-like [Halyomorpha halys] 393 aa protein | XP_014275003.1 GI: 939629564 |
| odorant receptor 7a-like [Halyomorpha halys] 123 aa protein | XP_014273506.1 GI: 939629170 |
| odorant receptor 85b-like [Halyomorpha halys] 388 aa protein | XP_014273330.1 GI: 939629125 |
| odorant receptor 85b-like [Halyomorpha halys] 386 aa protein | XP_014272535.1 GI: 939628833 |
| odorant receptor 24a-like [Halyomorpha halys] 409 aa protein | XP_014272447.1 GI: 939628818 |
| odorant receptor 4-like [Halyomorpha halys] 410 aa protein | XP_014294765.1 GI: 939628125 |
| odorant receptor 43b-like [Bactrocera oleae] 255 aa protein | XP_014101582.1 GI: 929382187 |
| odorant receptor 43b-like, partial [Bactrocera oleae] 256 aa protein | XP_014101520.1 GI: 929382078 |
| odorant receptor 88a-like, partial [Bactrocera oleae] 400 aa protein | XP_014101401.1 GI: 929381869 |
| odorant receptor 85c-like, partial [Bactrocera oleae] 277 aa protein | XP_014101291.1 GI: 929381677 |
| odorant receptor 67c-like [Bactrocera oleae] 405 aa protein | XP_014101001.1 GI: 929381148 |
| odorant receptor 43b-like, partial [Bactrocera oleae] 210 aa protein | XP_014100962.1 GI: 929381076 |
| odorant receptor 7a-like [Bactrocera oleae] 471 aa protein | XP_014100884.1 GI: 929380937 |
| odorant receptor 7a-like, partial [Bactrocera oleae] 398 aa protein | XP_014100883.1 GI: 929380935 |
| odorant receptor 59a-like [Bactrocera oleae] 249 aa protein | XP_014100068.1 GI: 929379483 |
| odorant receptor 30a-like [Bactrocera oleae] 403 aa protein | XP_014100035.1 GI: 929379425 |
| odorant receptor 88a-like [Bactrocera oleae] 410 aa protein | XP_014099351.1 GI: 929378168 |
| odorant receptor 88a-like [Bactrocera oleae] 404 aa protein | XP_014099350.1 GI: 929378166 |
| odorant receptor 42b-like [Bactrocera oleae] 256 aa protein | XP_014098809.1 GI: 929377189 |
| odorant receptor 63a-like [Bactrocera oleae] 415 aa protein | XP_014098250.1 GI: 929376191 |
| odorant receptor 45a-like [Bactrocera oleae] 365 aa protein | XP_014098074.1 GI: 929375879 |
| odorant receptor 45a-like, partial [Bactrocera oleae] 196 aa protein | XP_014098072.1 GI: 929375875 |
| odorant receptor 45a-like [Bactrocera oleae] 334 aa protein | XP_014098071.1 GI: 929375873 |
| odorant receptor la-like [Bactrocera oleae] 388 aa protein | XP_014098069.1 GI: 929375871 |
| odorant receptor 67d-like [Bactrocera oleae] 168 aa protein | XP_014097995.1 GI: 929375735 |
| odorant receptor Or2-like [Bactrocera oleae] 375 aa protein | XP_014097486.1 GI: 929374815 |
| odorant receptor Or2-like [Bactrocera oleae] 331 aa protein | XP_014097484.1 GI: 929374811 |
| odorant receptor 33b-like [Bactrocera oleae] 384 aa protein | XP_014097127.1 GI: 929374157 |
| odorant receptor 63a-like [Bactrocera oleae] 417 aa protein | XP_014096877.1 GI: 929373704 |
| odorant receptor 7a-like [Bactrocera oleae] 392 aa protein | XP_014095883.1 GI: 929371891 |
| odorant receptor 46a, isoform A [Bactrocera oleae] 388 aa protein | XP_014095654.1 GI: 929371469 |
| odorant receptor 49a-like [Bactrocera oleae] 394 aa protein | XP_014095510.1 GI: 929371206 |
| odorant receptor 94a-like [Bactrocera oleae] 337 aa protein | XP_014095326.1 GI: 929370867 |
| odorant receptor 67c [Bactrocera oleae] 404 aa protein | XP_014094968.1 GI: 929370189 |
| odorant receptor 94a-like [Bactrocera oleae] 403 aa protein | XP_014094554.1 GI: 929369423 |
| odorant receptor 94a-like [Bactrocera oleae] 392 aa protein | XP_014094548.1 GI: 929369411 |
| putative odorant receptor 85e [Bactrocera oleae] 450 aa protein | XP_014094455.1 GI: 929369240 |
| odorant receptor 13a [Bactrocera oleae] 456 aa protein | XP_014094420.1 GI: 929369174 |
| odorant receptor 63a-like [Bactrocera oleae] 275 aa protein | XP_014094225.1 GI: 929368815 |
| odorant receptor 63a-like [Bactrocera oleae] 414 aa protein | XP_014094224.1 GI: 929368813 |
| odorant receptor 63a-like [Bactrocera oleae] 357 aa protein | XP_014094223.1 GI: 929368811 |
| odorant receptor 85c-like [Bactrocera oleae] 415 aa protein | XP_014093775.1 GI: 929367999 |
| odorant receptor 85c-like [Bactrocera oleae] 211 aa protein | XP_014093774.1 GI: 929367997 |
| putative odorant receptor 85d [Bactrocera oleae] 420 aa protein | XP_014093772.1 GI: 929367993 |
| odorant receptor 7a [Bactrocera oleae] 396 aa protein | XP_014092478.1 GI: 929365645 |
| odorant receptor 94a-like [Bactrocera oleae] 192 aa protein | XP_014092468.1 GI: 929365629 |
| odorant receptor coreceptor [Bactrocera oleae] 473 aa protein | XP_014092453.1 GI: 929365601 |
| odorant receptor 83a [Bactrocera oleae] 473 aa protein | XP_014092452.1 GI: 929365599 |
| odorant receptor 7a-like [Bactrocera oleae] 394 aa protein | XP_014092042.1 GI: 929364851 |
| odorant receptor 10a [Bactrocera oleae] 402 aa protein | XP_014091911.1 GI: 929364611 |
| odorant receptor 82a [Bactrocera oleae] 398 aa protein | XP_014091900.1 GI: 929364589 |
| odorant receptor 67d-like [Bactrocera oleae] 388 aa protein | XP_014091805.1 GI: 929364416 |
| odorant receptor 67d-like [Bactrocera oleae] 388 aa protein | XP_014091792.1 GI: 929364391 |
| odorant receptor 74a [Bactrocera oleae] 402 aa protein | XP_014091648.1 GI: 929364123 |
| odorant receptor 2a-like [Bactrocera oleae] 393 aa protein | XP_014088938.1 GI: 929359208 |
| odorant receptor 94b-like [Bactrocera oleae] 402 aa protein | XP_014088795.1 GI: 929358946 |
| odorant receptor 43a [Bactrocera oleae] 378 aa protein | XP_014088559.1 GI: 929358514 |
| putative odorant receptor 69a, isoform B [Bactrocera oleae] 414 aa | XP_014088528.1 GI: 929358458 |
| protein | |
| putative odorant receptor 69a, isoform A [Bactrocera oleae] 289 aa | XP_014088513.1 GI: 929358429 |
| protein | |
| odorant receptor 43b-like [Bactrocera oleae] 337 aa protein | XP_014086206.1 GI: 929354211 |
| odorant receptor 74a-like [Bactrocera oleae] 414 aa protein | XP_014085775.1 GI: 929353423 |
| odorant receptor 35a-like [Bactrocera oleae] 417 aa protein | XP_014103705.1 GI: 929352035 |
| putative odorant receptor 92a [Bactrocera oleae] 250 aa protein | XP_014103608.1 GI: 929351857 |
| odorant receptor 7a-like [Bactrocera oleae] 384 aa protein | XP_014103552.1 GI: 929351755 |
| odorant receptor 2a-like [Bactrocera oleae] 384 aa protein | XP_014103551.1 GI: 929351753 |
| odorant receptor 85a-like [Bactrocera oleae] 254 aa protein | XP_014103550.1 GI: 929351751 |
| odorant receptor 22c [Bactrocera oleae] 400 aa protein | XP_014103181.1 GI: 929351088 |
| odorant receptor 24a [Bactrocera oleae] 397 aa protein | XP_014103094.1 GI: 929350935 |
| odorant receptor 49b-like [Bactrocera oleae] 371 aa protein | XP_014102906.1 GI: 929345515 |
| odorant receptor 47b [Bactrocera oleae] 423 aa protein | XP_014096040.1 GI: 929345200 |
| odorant receptor 59a-like [Bactrocera oleae] 378 aa protein | XP_014097365.1 GI: 929344966 |
| odorant receptor 59a-like [Bactrocera oleae] 380 aa protein | XP_014096236.1 GI: 929344964 |
| odorant receptor 53 [Microplitis mediator] 387 aa protein | AKO90017.1 GI: 861722551 |
| odorant receptor 52 [Microplitis mediator] 406 aa protein | AKO90016.1 GI: 861722548 |
| odorant receptor 51 [Microplitis mediator] 410 aa protein | AKO90015.1 GI: 861722545 |
| odorant receptor 50 [Microplitis mediator] 395 aa protein | AKO90014.1 GI: 861722542 |
| odorant receptor 49 [Microplitis mediator] 393 aa protein | AKO90013.1 GI: 861722539 |
| odorant receptor 48 [Microplitis mediator] 401 aa protein | AKO90012.1 GI: 861722536 |
| odorant receptor 47 [Microplitis mediator] 404 aa protein | AKO90011.1 GI: 861722533 |
| odorant receptor 46 [Microplitis mediator] 423 aa protein | AKO90010.1 GI: 861722530 |
| odorant receptor 45 [Microplitis mediator] 393 aa protein | AKO90009.1 GI: 861722527 |
| odorant receptor 44 [Microplitis mediator] 395 aa protein | AKO90008.1 GI: 861722524 |
| odorant receptor 43 [Microplitis mediator] 377 aa protein | AKO90007.1 GI: 861722521 |
| odorant receptor 42 [Microplitis mediator] 381 aa protein | AKO90006.1 GI: 861722518 |
| odorant receptor 41 [Microplitis mediator] 384 aa protein | AKO90005.1 GI: 861722515 |
| odorant receptor 40 [Microplitis mediator] 406 aa protein | AKO90004.1 GI: 861722506 |
| odorant receptor 39 [Microplitis mediator] 349 aa protein | AKO90003.1 GI: 861722503 |
| odorant receptor 38 [Microplitis mediator] 388 aa protein | AKO90002.1 GI: 861722500 |
| odorant receptor 37 [Microplitis mediator] 384 aa protein | AKO90001.1 GI: 861722497 |
| odorant receptor 36 [Microplitis mediator] 382 aa protein | AKO90000.1 GI: 861722494 |
| odorant receptor 35 [Microplitis mediator] 377 aa protein | AKO89999.1 GI: 861722490 |
| odorant receptor 34 [Microplitis mediator] 395 aa protein | AKO89998.1 GI: 861722487 |
| odorant receptor 33 [Microplitis mediator] 384 aa protein | AKO89997.1 GI: 861722484 |
| odorant receptor 32 [Microplitis mediator] 420 aa protein | AKO89996.1 GI: 861722481 |
| odorant receptor 31 [Microplitis mediator] 375 aa protein | AKO89995.1 GI: 861722478 |
| odorant receptor 30 [Microplitis mediator] 383 aa protein | AKO89994.1 GI: 861722475 |
| odorant receptor 29 [Microplitis mediator] 413 aa protein | AKO89993.1 GI: 861722471 |
| odorant receptor 28 [Microplitis mediator] 402 aa protein | AKO89992.1 GI: 861722468 |
| odorant receptor 27 [Microplitis mediator] 403 aa protein | AKO89991.1 GI: 861722465 |
| odorant receptor 26 [Microplitis mediator] 397 aa protein | AKO89990.1 GI: 861722462 |
| odorant receptor 25 [Microplitis mediator] 376 aa protein | AKO89989.1 GI: 861722459 |
| odorant receptor 24 [Microplitis mediator] 260 aa protein | AKO89988.1 GI: 861722456 |
| odorant receptor 23 [Microplitis mediator] 373 aa protein | AKO89987.1 GI: 861722453 |
| odorant receptor 22 [Microplitis mediator] 369 aa protein | AKO89986.1 GI: 861722450 |
| odorant receptor 21 [Microplitis mediator] 392 aa protein | AKO89985.1 GI: 861722447 |
| odorant receptor 20 [Microplitis mediator] 372 aa protein | AKO89984.1 GI: 861722444 |
| odorant receptor 19 [Microplitis mediator] 396 aa protein | AKO89983.1 GI: 861722441 |
| odorant receptor 18 [Microplitis mediator] 390 aa protein | AKO89982.1 GI: 861722438 |
| odorant receptor 17 [Microplitis mediator] 402 aa protein | AKO89981.1 GI: 861722435 |
| odorant receptor 16 [Microplitis mediator] 378 aa protein | AKO89980.1 GI: 861722432 |
| odorant receptor 15 [Microplitis mediator] 385 aa protein | AKO89979.1 GI: 861722429 |
| odorant receptor 14 [Microplitis mediator] 411 aa protein | AKO89978.1 GI: 861722426 |
| odorant receptor 13a-like [Plutella xylostella] 404 aa protein | NP_001296037.1 GI: 822092756 |
| odorant receptor 83b [Spodoptera litura] 473 aa protein | AFN22085.1 GI: 393757441 |
| odorant receptor 50 [Nasonia vitripennis] 373 aa protein | NP_001177496.1 GI: 299522706 |
| Gustatory and odorant receptor 21a 454 aa protein | Q9VPT1.3 GI: 158523347 |
| Gustatory and odorant receptor 63a 512 aa protein | Q9VZL7.1 GI: 20454944 |
| odorant receptor OR83b, partial [Chilo suppressalis] 338 aa | ACJ07125.1 GI: 210108262 |
| protein | |
| odorant receptor OR83b, partial [Sesamia inferens] 275 aa protein | ACJ07124.1 GI: 210108191 |
| odorant receptor coreceptor-like [Diuraphis noxia] 127 aa protein | XP_015378374.1 GI: 985425779 |
| gustatory and odorant receptor 63a-like [Halyomorpha halys] 403 | XP_014293240.1 GI: 939698138 |
| aa protein | |
| gustatory and odorant receptor 24-like isoform X2 [Halyomorpha | XP_014282243.1 GI: 939671105 |
| halys] 395 aa protein | |
| gustatory and odorant receptor 63a-like [Halyomorpha halys] 374 | XP_014281820.1 GI: 939670013 |
| aa protein | |
| gustatory and odorant receptor 63a-like [Halyomorpha halys] 395 | XP_014281153.1 GI: 939668267 |
| aa protein | |
| gustatory and odorant receptor 22-like [Halyomorpha halys] 134 | XP_014272022.1 GI: 939643445 |
| aa protein | |
| gustatory and odorant receptor 22-like [Halyomorpha halys] 199 | XP_014271847.1 GI: 939642935 |
| aa protein | |
| gustatory and odorant receptor 22-like isoform X2 [Halyomorpha | XP_014271842.1 GI: 939642921 |
| halys] 312 aa protein | |
| gustatory and odorant receptor 24-like isoform X1 [Halyomorpha | XP_014271841.1 GI: 939642918 |
| halys] 395 aa protein | |
| gustatory and odorant receptor 24-like isoform X4 [Halyomorpha | XP_014271840.1 GI: 939642916 |
| halys] 316 aa protein | |
| gustatory and odorant receptor 24-like isoform X3 [Halyomorpha | XP_014271839.1 GI: 939642913 |
| halys] 366 aa protein | |
| gustatory and odorant receptor 24-like isoform X2 [Halyomorpha | XP_014271838.1 GI: 939642910 |
| halys] 395 aa protein | |
| gustatory and odorant receptor 24-like isoform X1 [Halyomorpha | XP_014271836.1 GI: 939642908 |
| halys] 399 aa protein | |
| gustatory and odorant receptor 21a-like [Bactrocera oleae] 445 aa | XP_014101222.1 GI: 929381551 |
| protein | |
| gustatory and odorant receptor 21a-like, partial [Bactrocera oleae] | XP_014101212.1 GI: 929381535 |
| 367 aa protein | |
| gustatory and odorant receptor 21a-like, partial [Bactrocera oleae] | XP_014101173.1 GI: 929381464 |
| 278 aa protein | |
| gustatory and odorant receptor 21a-like, partial [Bactrocera oleae] | XP_014100623.1 GI: 929380462 |
| 200 aa protein | |
| gustatory and odorant receptor 21a [Bactrocera oleae] 456 aa | XP_014097799.1 GI: 929375381 |
| protein | |
| gustatory and odorant receptor 22-like [Bactrocera oleae] 129 aa | XP_014097326.1 GI: 929374521 |
| protein | |
| odorant receptor 67c-like, partial [Bactrocera oleae] 235 aa protein | XP_014096202.1 GI: 929372471 |
| gustatory and odorant receptor 63a [Bactrocera oleae] 485 aa | XP_014095104.1 GI: 929347538 |
| protein | |
| odorant receptor Or83b [Helicoverpa zea] 472 aa protein | AAX14773.1 GI: 60207120 |
| odorant receptor Or83b [Ceratitis capitata] 473 aa protein | AAX14775.1 GI: 60207191 |
| odorant receptor Or83b [Anopheles gambiae] 478 aa protein | AAX14774.1 GI: 60207155 |
| putative odorant receptor [Bombyx mori] 472 aa protein | BAD69585.1 GI: 55583295 |
| Gustatory and odorant receptor 24 457 aa protein | Q7PYF4.4 GI: 384872698 |
| Gustatory and odorant receptor 22 467 aa protein | Q7PMG3.1 GI: 74799392 |
| odorant receptor 7 [Culex pipiens pallens] 480 aa protein | AMQ13062.1 GI: 1005652053 |
| putative odorant receptor [Sesamia inferens] 442 aa protein | AGY14587.2 GI: 670657525 |
| putative odorant receptor [Sesamia inferens] 426 aa protein | AGY14585.2 GI: 670657520 |
| putative odorant receptor [Sesamia inferens] 432 aa protein | AGY14579.2 GI: 670657516 |
| putative odorant receptor [Sesamia inferens] 473 aa protein | AGY14565.1 GI: 550848914 |
| putative odorant receptor SinfOR18, partial [Sesamia inferens] 153 | AIF79425.1 GI: 665823788 |
| aa protein | |
| putative odorant receptor SinfOR17, partial [Sesamia inferens] 82 | AIF79424.1 GI: 665823786 |
| aa protein | |
| putative odorant receptor, partial [Sesamia inferens] 162 aa protein | AGY14595.1 GI: 550848974 |
| putative odorant receptor [Sesamia inferens] 402 aa protein | AGY14593.1 GI: 550848970 |
| putative odorant receptor, partial [Sesamia inferens] 225 aa protein | AGY14592.1 GI: 550848968 |
| putative odorant receptor, partial [Sesamia inferens] 230 aa protein | AGY14591.1 GI: 550848966 |
| putative odorant receptor, partial [Sesamia inferens] 380 aa protein | AGY14590.1 GI: 550848964 |
| putative odorant receptor, partial [Sesamia inferens] 250 aa protein | AGY14589.1 GI: 550848962 |
| putative odorant receptor, partial [Sesamia inferens] 275 aa protein | AGY14588.1 GI: 550848960 |
| putative odorant receptor, partial [Sesamia inferens] 268 aa protein | AGY14586.1 GI: 550848956 |
| putative odorant receptor, partial [Sesamia inferens] 203 aa protein | AGY14584.1 GI: 550848952 |
| putative odorant receptor, partial [Sesamia inferens] 223 aa protein | AGY14583.1 GI: 550848950 |
| putative odorant receptor, partial [Sesamia inferens] 247 aa protein | AGY14582.1 GI: 550848948 |
| putative odorant receptor, partial [Sesamia inferens] 254 aa protein | AGY14581.1 GI: 550848946 |
| putative odorant receptor, partial [Sesamia inferens] 224 aa protein | AGY14578.1 GI: 550848940 |
| putative odorant receptor, partial [Sesamia inferens] 368 aa protein | AGY14577.1 GI: 550848938 |
| putative odorant receptor, partial [Sesamia inferens] 67 aa protein | AGY14576.1 GI: 550848936 |
| putative odorant receptor, partial [Sesamia inferens] 88 aa protein | AGY14575.1 GI: 550848934 |
| putative odorant receptor, partial [Sesamia inferens] 84 aa protein | AGY14574.1 GI: 550848932 |
| putative odorant receptor, partial [Sesamia inferens] 69 aa protein | AGY14573.1 GI: 550848930 |
| putative odorant receptor, partial [Sesamia inferens] 118 aa protein | AGY14572.1 GI: 550848928 |
| putative odorant receptor, partial [Sesamia inferens] 95 aa protein | AGY14571.1 GI: 550848926 |
| putative odorant receptor, partial [Sesamia inferens] 161 aa protein | AGY14570.1 GI: 550848924 |
| putative odorant receptor, partial [Sesamia inferens] 82 aa protein | AGY14568.1 GI: 550848920 |
| putative odorant receptor, partial [Sesamia inferens] 115 aa protein | AGY14567.1 GI: 550848918 |
| putative odorant receptor, partial [Sesamia inferens] 98 aa protein | AGY14566.1 GI: 550848916 |
| odorant receptor 7 [Plutella xylostella] 424 aa protein | AGK43829.1 GI: 484354001 |
| odorant receptor 6 [Plutella xylostella] 424 aa protein | AGK43828.1 GI: 484353999 |
| odorant receptor 5 [Plutella xylostella] 404 aa protein | AGK43827.1 GI: 484353997 |
| odorant receptor 4 [Plutella xylostella] 402 aa protein | AGK43826.1 GI: 484353995 |
| odorant receptor 3 [Plutella xylostella] 403 aa protein | AGK43825.1 GI: 484353993 |
| odorant receptor 1 [Plutella xylostella] 422 aa protein | AGK43824.1 GI: 484353991 |
| odorant receptor 2 [Cnaphalocrocis medinalis] 473 aa protein | AFG73001.1 GI: 383215098 |
| putative odorant receptor [Bombyx mori] 430 aa protein | BAD69586.1 GI: 55583297 |
| putative odorant receptor, partial [Reticulitermes speratus] 382 aa | BAU20249.1 GI: 966774588 |
| protein | |
| putative odorant receptor [Reticulitermes speratus] 199 aa protein | BAU20248.1 GI: 966774586 |
| putative odorant receptor [Reticulitermes speratus] 293 aa protein | BAU20247.1 GI: 966774584 |
| putative odorant receptor, partial [Reticulitermes speratus] 125 aa | BAU20246.1 GI: 966774582 |
| protein | |
| putative odorant receptor [Reticulitermes speratus] 481 aa protein | BAU20245.1 GI: 966774580 |
| putative odorant receptor [Reticulitermes speratus] 218 aa protein | BAU20244.1 GI: 966774578 |
| putative odorant receptor, partial [Reticulitermes speratus] 119 aa | BAU20243.1 GI: 966774576 |
| protein | |
| putative odorant receptor, partial [Reticulitermes speratus] 240 aa | BAU20242.1 GI: 966774574 |
| protein | |
| putative odorant receptor, partial [Reticulitermes speratus] 359 aa | BAU20241.1 GI: 966774572 |
| protein | |
| putative odorant receptor co-receptor [Reticulitermes speratus] 472 | BAU20240.1 GI: 966774570 |
| aa protein | |
| putative odorant receptor, partial [Reticulitermes speratus] 224 aa | BAU20239.1 GI: 966774568 |
| protein | |
| putative odorant receptor, partial [Reticulitermes speratus] 235 aa | BAU20238.1 GI: 966774566 |
| protein | |
| putative odorant receptor [Reticulitermes speratus] 491 aa protein | BAU20237.1 GI: 966774564 |
| putative odorant receptor [Reticulitermes speratus] 461 aa protein | BAU20236.1 GI: 966774562 |
| putative odorant receptor, partial [Reticulitermes speratus] 211 aa | BAU20235.1 GI: 966774560 |
| protein | |
| putative odorant receptor [Reticulitermes speratus] 406 aa protein | BAU20234.1 GI: 966774558 |
| putative odorant receptor, partial [Reticulitermes speratus] 411 aa | BAU20233.1 GI: 966774556 |
| protein | |
| putative odorant receptor, partial [Reticulitermes speratus] 469 aa | BAU20232.1 GI: 966774554 |
| protein | |
| putative odorant receptor, partial [Reticulitermes speratus] 334 aa | BAU20231.1 GI: 966774552 |
| protein | |
| putative odorant receptor 3, partial [Reticulitermes speratus] 429 | BAU20230.1 GI: 966774550 |
| aa protein | |
| putative odorant receptor 2 [Reticulitermes speratus] 153 aa | BAU20229.1 GI: 966774548 |
| protein | |
| putative odorant receptor 1, partial [Reticulitermes speratus] 101 | BAU20228.1 GI: 966774546 |
| aa protein | |
| putative odorant receptor, partial [Sesamia inferens] 156 aa protein | AGY14594.1 GI: 550848972 |
| putative odorant receptor, partial [Sesamia inferens] 107 aa protein | AGY14580.1 GI: 550848944 |
| putative odorant receptor, partial [Sesamia inferens] 70 aa protein | AGY14569.1 GI: 550848922 |
| putative odorant receptor, partial [Sesamia inferens] 188 aa protein | AGY14564.1 GI: 550848912 |
| Odorant receptor coreceptor; Odorant receptor 83b 486 aa protein | Q9VNB5.2 GI: 14285640 |
| Odorant receptor 22a 397 aa protein | P81909.1 GI: 12643687 |
| Odorant receptor 59b 398 aa protein | Q9W1P8.1 GI: 11387003 |
| Odorant receptor 67a 407 aa protein | Q9VT08.2 GI: 14285630 |
| Odorant receptor 43a 376 aa protein | P81917.2 GI: 12643691 |
| Odorant receptor 22b 397 aa protein | P81910.3 GI: 221222515 |
| Odorant receptor 35a 409 aa protein | Q9V3Q2.3 GI: 48429266 |
| Odorant receptor 67d 391 aa protein | Q9VT92.3 GI: 47117341 |
| Odorant receptor 46a, isoform A 381 aa protein | P81919.4 GI: 39932724 |
| Odorant receptor 85b 390 aa protein | Q9VHQ7.2 GI: 14285638 |
| Odorant receptor 67b 421 aa protein | Q9VT20.2 GI: 14285631 |
| Odorant receptor 46a, isoform B 384 aa protein | Q9V3N2.2 GI: 14285623 |
| Odorant receptor 47a 385 aa protein | P81921.1 GI: 12643694 |
| Odorant receptor 10a 406 aa protein | Q9VYZ1.1 GI: 11387000 |
| Odorant receptor 24a 398 aa protein | P81913.4 GI: 251757500 |
| Odorant receptor 47b 412 aa protein | P81922.2 GI: 47606742 |
| Odorant receptor 30a 377 aa protein | Q9VLE5.4 GI: 41019523 |
| Odorant receptor 45a 378 aa protein | Q9V568.3 GI: 37999962 |
| Odorant receptor 42a 406 aa protein | Q9V9I2.3 GI: 22096371 |
| Odorant receptor 49a 396 aa protein | Q9V6A9.3 GI: 22096370 |
| Odorant receptor 1a 392 aa protein | Q9W5G6.2 GI: 14285651 |
| Odorant receptor 13a 418 aa protein | Q9VXL0.2 GI: 14285650 |
| Odorant receptor 88a 401 aa protein | Q9VFN2.2 GI: 14285649 |
| Odorant receptor 98a 397 aa protein | Q9VAZ3.2 GI: 14285647 |
| Odorant receptor 19a 387 aa protein | Q9I816.2 GI: 14285645 |
| Odorant receptor 83a 453 aa protein | Q9VNB3.2 GI: 14285639 |
| Odorant receptor 85c 389 aa protein | Q9VHQ6.2 GI: 14285637 |
| Odorant receptor 85f 392 aa protein | Q9VHE6.1 GI: 14285636 |
| Odorant receptor 82a 385 aa protein | P82986.1 GI: 14285635 |
| Odorant receptor 63a 420 aa protein | Q9VZW8.2 GI: 14285633 |
| Odorant receptor 67c 404 aa protein | Q9VT90.2 GI: 14285632 |
| Odorant receptor 42b 399 aa protein | Q9V9I4.2 GI: 14285628 |
| Odorant receptor 56a 419 aa protein | Q9V8Y7.2 GI: 14285626 |
| Odorant receptor 65a 417 aa protein | P82982.1 GI: 14285620 |
| Odorant receptor 43b 403 aa protein | P81918.3 GI: 14285618 |
| Odorant receptor 22c 402 aa protein | P81911.2 GI: 14285616 |
| Odorant receptor 49b 375 aa protein | Q9V6H2.1 GI: 12643916 |
| Odorant receptor 59a 397 aa protein | P81923.2 GI: 12643696 |
| Odorant receptor 2a 397 aa protein | O46077.2 GI: 12643564 |
| Odorant receptor 7a 413 aa protein | Q9W3I5.1 GI: 11387005 |
| Odorant receptor 9a 392 aa protein | Q9W2U9.1 GI: 11387004 |
| Odorant receptor 74a 404 aa protein | Q9VVF3.1 GI: 11386999 |
| Odorant receptor 85a 397 aa protein | Q9VHS4.1 GI: 11386993 |
| Odorant receptor 94a 387 aa protein | Q9VCS9.1 GI: 11386991 |
| Odorant receptor 94b 383 aa protein | Q9VCS8.1 GI: 11386990 |
| Odorant receptor 45b 396 aa protein | Q9V589.1 GI: 11386985 |
| Odorant receptor 33c 384 aa protein | P81916.1 GI: 11386980 |
| Odorant receptor 33b 379 aa protein | P81915.1 GI: 11386979 |
| Odorant receptor 33a 378 aa protein | P81914.1 GI: 11386978 |
| Odorant receptor 23a 379 aa protein | P81912.1 GI: 11386977 |
| hypothetical protein X777_04609 [Cerapachys biroi] 397 aa | EZA62900.1 GI: 607368794 |
| protein | |
| hypothetical protein X777_04608 [Cerapachys biroi] 399 aa | EZA62899.1 GI: 607368793 |
| protein | |
| hypothetical protein X777_07630, partial [Cerapachys biroi] 109 | EZA62813.1 GI: 607368701 |
| aa protein | |
| hypothetical protein X777_07612, partial [Cerapachys biroi] 305 | EZA62796.1 GI: 607368684 |
| aa protein | |
| hypothetical protein X777_07611 [Cerapachys biroi] 376 aa | EZA62795.1 GI: 607368683 |
| protein | |
| hypothetical protein X777_10236 [Cerapachys biroi] 402 aa | EZA62605.1 GI: 607368492 |
| protein | |
| hypothetical protein X777_03408 [Cerapachys biroi] 389 aa | EZA62373.1 GI: 607368259 |
| protein | |
| hypothetical protein X777_03407 [Cerapachys biroi] 390 aa | EZA62372.1 GI: 607368258 |
| protein | |
| hypothetical protein X777_03406 [Cerapachys biroi] 391 aa | EZA62371.1 GI: 607368257 |
| protein | |
| hypothetical protein X777_03405 [Cerapachys biroi] 391 aa | EZA62370.1 GI: 607368256 |
| protein | |
| hypothetical protein X777_03404 [Cerapachys biroi] 382 aa | EZA62369.1 GI: 607368255 |
| protein | |
| hypothetical protein X777_03402 [Cerapachys biroi] 390 aa | EZA62368.1 GI: 607368254 |
| protein | |
| hypothetical protein X777_03401 [Cerapachys biroi] 392 aa | EZA62367.1 GI: 607368253 |
| protein | |
| hypothetical protein X777_09391 [Cerapachys biroi] 395 aa | EZA61770.1 GI: 607367631 |
| protein | |
| hypothetical protein X777_09390 [Cerapachys biroi] 396 aa | EZA61769.1 GI: 607367630 |
| protein | |
| hypothetical protein X777_09389 [Cerapachys biroi] 399 aa | EZA61768.1 GI: 607367629 |
| protein | |
| hypothetical protein X777_09388 [Cerapachys biroi] 394 aa | EZA61767.1 GI: 607367628 |
| protein | |
| hypothetical protein X777_09387 [Cerapachys biroi] 397 aa | EZA61766.1 GI: 607367627 |
| protein | |
| hypothetical protein X777_09305 [Cerapachys biroi] 394 aa | EZA61684.1 GI: 607367545 |
| protein | |
| hypothetical protein X777_07969 [Cerapachys biroi] 350 aa | EZA61634.1 GI: 607367487 |
| protein | |
| hypothetical protein X777_07966 [Cerapachys biroi] 396 aa | EZA61633.1 GI: 607367486 |
| protein | |
| hypothetical protein X777_07964 [Cerapachys biroi] 395 aa | EZA61631.1 GI: 607367484 |
| protein | |
| hypothetical protein X777_12474 [Cerapachys biroi] 375 aa | EZA61376.1 GI: 607367226 |
| protein | |
| hypothetical protein X777_08255 [Cerapachys biroi] 397 aa | EZA61043.1 GI: 607366887 |
| protein | |
| hypothetical protein X777_14254, partial [Cerapachys biroi] 255 | EZA60648.1 GI: 607366483 |
| aa protein | |
| hypothetical protein X777_13392 [Cerapachys biroi] 417 aa | EZA60303.1 GI: 607366132 |
| protein | |
| hypothetical protein X777_16159 [Cerapachys biroi] 396 aa | EZA59956.1 GI: 607365776 |
| protein | |
| hypothetical protein X777_16072 [Cerapachys biroi] 382 aa | EZA59870.1 GI: 607365690 |
| protein | |
| hypothetical protein X777_00307 [Cerapachys biroi] 382 aa | EZA59464.1 GI: 607365266 |
| protein | |
| hypothetical protein X777_00306 [Cerapachys biroi] 382 aa | EZA59463.1 GI: 607365265 |
| protein | |
| hypothetical protein X777_00305 [Cerapachys biroi] 379 aa | EZA59462.1 GI: 607365264 |
| protein | |
| hypothetical protein X777_00304 [Cerapachys biroi] 379 aa | EZA59461.1 GI: 607365263 |
| protein | |
| hypothetical protein X777_00303 [Cerapachys biroi] 380 aa | EZA59460.1 GI: 607365262 |
| protein | |
| hypothetical protein X777_00693, partial [Cerapachys biroi] 388 | EZA58872.1 GI: 607364664 |
| aa protein | |
| hypothetical protein X777_00565, partial [Cerapachys biroi] 110 | EZA58870.1 GI: 607364661 |
| aa protein | |
| hypothetical protein X777_00710, partial [Cerapachys biroi] 374 | EZA58868.1 GI: 607364658 |
| aa protein | |
| hypothetical protein X777_00709, partial [Cerapachys biroi] 393 | EZA58867.1 GI: 607364657 |
| aa protein | |
| hypothetical protein X777_00705, partial [Cerapachys biroi] 391 | EZA58866.1 GI: 607364656 |
| aa protein | |
| hypothetical protein X777_00703, partial [Cerapachys biroi] 390 | EZA58865.1 GI: 607364655 |
| aa protein | |
| hypothetical protein X777_00702, partial [Cerapachys biroi] 373 | EZA58864.1 GI: 607364654 |
| aa protein | |
| hypothetical protein X777_00701, partial [Cerapachys biroi] 390 | EZA58863.1 GI: 607364653 |
| aa protein | |
| hypothetical protein X777_00700, partial [Cerapachys biroi] 390 | EZA58862.1 GI: 607364652 |
| aa protein | |
| hypothetical protein X777_14780, partial [Cerapachys biroi] 298 | EZA58612.1 GI: 607364400 |
| aa protein | |
| hypothetical protein X777_14779 [Cerapachys biroi] 368 aa | EZA58611.1 GI: 607364399 |
| protein | |
| hypothetical protein X777_14777 [Cerapachys biroi] 368 aa | EZA58610.1 GI: 607364398 |
| protein | |
| hypothetical protein X777_14776 [Cerapachys biroi] 373 aa | EZA58609.1 GI: 607364397 |
| protein | |
| hypothetical protein X777_14775 [Cerapachys biroi] 374 aa | EZA58608.1 GI: 607364396 |
| protein | |
| hypothetical protein X777_14774 [Cerapachys biroi] 372 aa | EZA58607.1 GI: 607364395 |
| protein | |
| hypothetical protein X777_14773 [Cerapachys biroi] 367 aa | EZA58606.1 GI: 607364394 |
| protein | |
| hypothetical protein X777_14772 [Cerapachys biroi] 372 aa | EZA58605.1 GI: 607364393 |
| protein | |
| hypothetical protein X777_14770 [Cerapachys biroi] 370 aa | EZA58604.1 GI: 607364392 |
| protein | |
| hypothetical protein X777_14769 [Cerapachys biroi] 371 aa | EZA58603.1 GI: 607364391 |
| protein | |
| hypothetical protein X777_14768 [Cerapachys biroi] 369 aa | EZA58602.1 GI: 607364390 |
| protein | |
| hypothetical protein X777_14767 [Cerapachys biroi] 365 aa | EZA58601.1 GI: 607364389 |
| protein | |
| hypothetical protein X777_14766, partial [Cerapachys biroi] 362 | EZA58600.1 GI: 607364388 |
| aa protein | |
| hypothetical protein X777_01291 [Cerapachys biroi] 417 aa | EZA58334.1 GI: 607364113 |
| protein | |
| hypothetical protein X777_01236 [Cerapachys biroi] 391 aa | EZA58279.1 GI: 607364058 |
| protein | |
| hypothetical protein X777_01205 [Cerapachys biroi] 403 aa | EZA58248.1 GI: 607364027 |
| protein | |
| hypothetical protein X777_01932, partial [Cerapachys biroi] 212 | EZA58117.1 GI: 607363890 |
| aa protein | |
| hypothetical protein X777_01931, partial [Cerapachys biroi] 386 | EZA58116.1 GI: 607363889 |
| aa protein | |
| hypothetical protein X777_01929 [Cerapachys biroi] 387 aa | EZA58115.1 GI: 607363888 |
| protein | |
| hypothetical protein X777_01925, partial [Cerapachys biroi] 387 | EZA58113.1 GI: 607363886 |
| aa protein | |
| hypothetical protein X777_01917, partial [Cerapachys biroi] 388 | EZA58112.1 GI: 607363885 |
| aa protein | |
| hypothetical protein X777_01492, partial [Cerapachys biroi] 346 | EZA58111.1 GI: 607363883 |
| aa protein | |
| hypothetical protein X777_01491 [Cerapachys biroi] 394 aa | EZA58110.1 GI: 607363882 |
| protein | |
| hypothetical protein X777_01490 [Cerapachys biroi] 392 aa | EZA58109.1 GI: 607363881 |
| protein | |
| hypothetical protein X777_02093, partial [Cerapachys biroi] 368 | EZA57554.1 GI: 607363317 |
| aa protein | |
| hypothetical protein X777_02092 [Cerapachys biroi] 377 aa | EZA57553.1 GI: 607363316 |
| protein | |
| hypothetical protein X777_02090 [Cerapachys biroi] 395 aa | EZA57552.1 GI: 607363315 |
| protein | |
| hypothetical protein X777_02089 [Cerapachys biroi] 387 aa | EZA57551.1 GI: 607363314 |
| protein | |
| hypothetical protein X777_02088 [Cerapachys biroi] 393 aa | EZA57550.1 GI: 607363313 |
| protein | |
| hypothetical protein X777_02087 [Cerapachys biroi] 381 aa | EZA57549.1 GI: 607363312 |
| protein | |
| hypothetical protein X777_02086 [Cerapachys biroi] 378 aa | EZA57548.1 GI: 607363311 |
| protein | |
| hypothetical protein X777_02085 [Cerapachys biroi] 387 aa | EZA57547.1 GI: 607363310 |
| protein | |
| hypothetical protein X777_02082 [Cerapachys biroi] 396 aa | EZA57546.1 GI: 607363309 |
| protein | |
| hypothetical protein X777_02081 [Cerapachys biroi] 377 aa | EZA57545.1 GI: 607363308 |
| protein | |
| hypothetical protein X777_02080 [Cerapachys biroi] 385 aa | EZA57544.1 GI: 607363307 |
| protein | |
| hypothetical protein X777_02079 [Cerapachys biroi] 382 aa | EZA57543.1 GI: 607363306 |
| protein | |
| hypothetical protein X777_02078 [Cerapachys biroi] 384 aa | EZA57542.1 GI: 607363305 |
| protein | |
| hypothetical protein X777_02075 [Cerapachys biroi] 385 aa | EZA57540.1 GI: 607363303 |
| protein | |
| hypothetical protein X777_02074 [Cerapachys biroi] 387 aa | EZA57539.1 GI: 607363302 |
| protein | |
| hypothetical protein X777_02073 [Cerapachys biroi] 383 aa | EZA57538.1 GI: 607363301 |
| protein | |
| hypothetical protein X777_02072 [Cerapachys biroi] 385 aa | EZA57537.1 GI: 607363300 |
| protein | |
| hypothetical protein X777_02071 [Cerapachys biroi] 382 aa | EZA57536.1 GI: 607363299 |
| protein | |
| hypothetical protein X777_02070 [Cerapachys biroi] 384 aa | EZA57535.1 GI: 607363298 |
| protein | |
| hypothetical protein X777_02069, partial [Cerapachys biroi] 367 | EZA57534.1 GI: 607363297 |
| aa protein | |
| hypothetical protein X777_02068 [Cerapachys biroi] 384 aa | EZA57533.1 GI: 607363296 |
| protein | |
| hypothetical protein X777_02067 [Cerapachys biroi] 384 aa | EZA57532.1 GI: 607363295 |
| protein | |
| hypothetical protein X777_02065 [Cerapachys biroi] 379 aa | EZA57531.1 GI: 607363294 |
| protein | |
| hypothetical protein X777_01603, partial [Cerapachys biroi] 388 | EZA56997.1 GI: 607362752 |
| aa protein | |
| hypothetical protein X777_01602, partial [Cerapachys biroi] 390 | EZA56996.1 GI: 607362751 |
| aa protein | |
| hypothetical protein X777_01601, partial [Cerapachys biroi] 389 | EZA56995.1 GI: 607362750 |
| aa protein | |
| hypothetical protein X777_01600, partial [Cerapachys biroi] 379 | EZA56994.1 GI: 607362749 |
| aa protein | |
| hypothetical protein X777_02369 [Cerapachys biroi] 375 aa | EZA56762.1 GI: 607362510 |
| protein | |
| hypothetical protein X777_02368 [Cerapachys biroi] 368 aa | EZA56761.1 GI: 607362509 |
| protein | |
| hypothetical protein X777_02340 [Cerapachys biroi] 398 aa | EZA56735.1 GI: 607362483 |
| protein | |
| hypothetical protein X777_03347 [Cerapachys biroi] 391 aa | EZA56560.1 GI: 607362304 |
| protein | |
| hypothetical protein X777_03346, partial [Cerapachys biroi] 377 | EZA56559.1 GI: 607362303 |
| aa protein | |
| hypothetical protein X777_03345 [Cerapachys biroi] 392 aa | EZA56558.1 GI: 607362302 |
| protein | |
| hypothetical protein X777_03344 [Cerapachys biroi] 372 aa | EZA56557.1 GI: 607362301 |
| protein | |
| hypothetical protein X777_03736, partial [Cerapachys biroi] 383 | EZA56086.1 GI: 607361821 |
| aa protein | |
| hypothetical protein X777_04121 [Cerapachys biroi] 383 aa | EZA55902.1 GI: 607361626 |
| protein | |
| hypothetical protein X777_04120 [Cerapachys biroi] 383 aa | EZA55901.1 GI: 607361625 |
| protein | |
| hypothetical protein X777_03929 [Cerapachys biroi] 388 aa | EZA55755.1 GI: 607361477 |
| protein | |
| hypothetical protein X777_03928, partial [Cerapachys biroi] 335 | EZA55754.1 GI: 607361476 |
| aa protein | |
| hypothetical protein X777_04285 [Cerapachys biroi] 388 aa | EZA55491.1 GI: 607361197 |
| protein | |
| hypothetical protein X777_04264 [Cerapachys biroi] 396 aa | EZA55471.1 GI: 607361177 |
| protein | |
| hypothetical protein X777_04263 [Cerapachys biroi] 393 aa | EZA55470.1 GI: 607361176 |
| protein | |
| hypothetical protein X777_04768 [Cerapachys biroi] 299 aa | EZA55372.1 GI: 607361071 |
| protein | |
| hypothetical protein X777_04767 [Cerapachys biroi] 395 aa | EZA55371.1 GI: 607361070 |
| protein | |
| hypothetical protein X777_04765 [Cerapachys biroi] 412 aa | EZA55369.1 GI: 607361068 |
| protein | |
| hypothetical protein X777_05311 [Cerapachys biroi] 388 aa | EZA55133.1 GI: 607360814 |
| protein | |
| hypothetical protein X777_05310 [Cerapachys biroi] 391 aa | EZA55132.1 GI: 607360813 |
| protein | |
| hypothetical protein X777_05285, partial [Cerapachys biroi] 347 | EZA55110.1 GI: 607360791 |
| aa protein | |
| hypothetical protein X777_05369 [Cerapachys biroi] 395 aa | EZA55090.1 GI: 607360767 |
| protein | |
| hypothetical protein X777_05368 [Cerapachys biroi] 398 aa | EZA55089.1 GI: 607360766 |
| protein | |
| hypothetical protein X777_05462 [Cerapachys biroi] 391 aa | EZA54938.1 GI: 607360613 |
| protein | |
| hypothetical protein X777_05757 [Cerapachys biroi] 404 aa | EZA54481.1 GI: 607360141 |
| protein | |
| hypothetical protein X777_05686, partial [Cerapachys biroi] 347 | EZA54448.1 GI: 607360105 |
| aa protein | |
| hypothetical protein X777_05685 [Cerapachys biroi] 394 aa | EZA54447.1 GI: 607360104 |
| protein | |
| hypothetical protein X777_05682, partial [Cerapachys biroi] 344 | EZA54446.1 GI: 607360103 |
| aa protein | |
| hypothetical protein X777_05679, partial [Cerapachys biroi] 385 | EZA54444.1 GI: 607360101 |
| aa protein | |
| hypothetical protein X777_05674, partial [Cerapachys biroi] 346 | EZA54439.1 GI: 607360096 |
| aa protein | |
| hypothetical protein X777_05671, partial [Cerapachys biroi] 307 | EZA54437.1 GI: 607360094 |
| aa protein | |
| hypothetical protein X777_05670, partial [Cerapachys biroi] 86 aa | EZA54436.1 GI: 607360093 |
| protein | |
| hypothetical protein X777_05669 [Cerapachys biroi] 401 aa | EZA54435.1 GI: 607360092 |
| protein | |
| hypothetical protein X777_05668 [Cerapachys biroi] 393 aa | EZA54434.1 GI: 607360091 |
| protein | |
| hypothetical protein X777_05665 [Cerapachys biroi] 353 aa | EZA54432.1 GI: 607360089 |
| protein | |
| hypothetical protein X777_05664 [Cerapachys biroi] 393 aa | EZA54431.1 GI: 607360088 |
| protein | |
| hypothetical protein X777_05662 [Cerapachys biroi] 399 aa | EZA54429.1 GI: 607360086 |
| protein | |
| hypothetical protein X777_05661 [Cerapachys biroi] 396 aa | EZA54428.1 GI: 607360085 |
| protein | |
| hypothetical protein X777_05660 [Cerapachys biroi] 394 aa | EZA54427.1 GI: 607360084 |
| protein | |
| hypothetical protein X777_05658 [Cerapachys biroi] 394 aa | EZA54426.1 GI: 607360083 |
| protein | |
| hypothetical protein X777_05656, partial [Cerapachys biroi] 347 | EZA54424.1 GI: 607360081 |
| aa protein | |
| hypothetical protein X777_05655 [Cerapachys biroi] 396 aa | EZA54423.1 GI: 607360080 |
| protein | |
| hypothetical protein X777_05651 [Cerapachys biroi] 394 aa | EZA54421.1 GI: 607360078 |
| protein | |
| hypothetical protein X777_05650 [Cerapachys biroi] 394 aa | EZA54420.1 GI: 607360077 |
| protein | |
| hypothetical protein X777_05649 [Cerapachys biroi] 395 aa | EZA54419.1 GI: 607360076 |
| protein | |
| hypothetical protein X777_05648 [Cerapachys biroi] 394 aa | EZA54418.1 GI: 607360075 |
| protein | |
| hypothetical protein X777_07020, partial [Cerapachys biroi] 305 | EZA53552.1 GI: 607359176 |
| aa protein | |
| hypothetical protein X777_07019 [Cerapachys biroi] 352 aa | EZA53551.1 GI: 607359175 |
| protein | |
| hypothetical protein X777_08120 [Cerapachys biroi] 391 aa | EZA52637.1 GI: 607358221 |
| protein | |
| hypothetical protein X777_08119 [Cerapachys biroi] 389 aa | EZA52636.1 GI: 607358220 |
| protein | |
| hypothetical protein X777_08118 [Cerapachys biroi] 359 aa | EZA52635.1 GI: 607358219 |
| protein | |
| hypothetical protein X777_08117 [Cerapachys biroi] 354 aa | EZA52634.1 GI: 607358218 |
| protein | |
| hypothetical protein X777_08040 [Cerapachys biroi] 387 aa | EZA52558.1 GI: 607358142 |
| protein | |
| hypothetical protein X777_08039 [Cerapachys biroi] 398 aa | EZA52557.1 GI: 607358141 |
| protein | |
| hypothetical protein X777_08037 [Cerapachys biroi] 402 aa | EZA52556.1 GI: 607358140 |
| protein | |
| hypothetical protein X777_08034 [Cerapachys biroi] 387 aa | EZA52555.1 GI: 607358139 |
| protein | |
| hypothetical protein X777_08033 [Cerapachys biroi] 395 aa | EZA52554.1 GI: 607358138 |
| protein | |
| hypothetical protein X777_08032 [Cerapachys biroi] 404 aa | EZA52553.1 GI: 607358137 |
| protein | |
| hypothetical protein X777_08610 [Cerapachys biroi] 393 aa | EZA52503.1 GI: 607358081 |
| protein | |
| hypothetical protein X777_08609 [Cerapachys biroi] 393 aa | EZA52502.1 GI: 607358080 |
| protein | |
| hypothetical protein X777_08608, partial [Cerapachys biroi] 348 | EZA52501.1 GI: 607358079 |
| aa protein | |
| hypothetical protein X777_08641 [Cerapachys biroi] 403 aa | EZA52498.1 GI: 607358074 |
| protein | |
| hypothetical protein X777_08639 [Cerapachys biroi] 405 aa | EZA52497.1 GI: 607358073 |
| protein | |
| hypothetical protein X777_08638 [Cerapachys biroi] 415 aa | EZA52496.1 GI: 607358072 |
| protein | |
| hypothetical protein X777_08637 [Cerapachys biroi] 410 aa | EZA52495.1 GI: 607358071 |
| protein | |
| hypothetical protein X777_08636 [Cerapachys biroi] 387 aa | EZA52494.1 GI: 607358070 |
| protein | |
| hypothetical protein X777_08635 [Cerapachys biroi] 412 aa | EZA52493.1 GI: 607358069 |
| protein | |
| hypothetical protein X777_08634, partial [Cerapachys biroi] 296 | EZA52492.1 GI: 607358068 |
| aa protein | |
| hypothetical protein X777_08633, partial [Cerapachys biroi] 296 | EZA52491.1 GI: 607358067 |
| aa protein | |
| hypothetical protein X777_08535 [Cerapachys biroi] 369 aa | EZA52460.1 GI: 607358030 |
| protein | |
| hypothetical protein X777_08647 [Cerapachys biroi] 409 aa | EZA52136.1 GI: 607357695 |
| protein | |
| hypothetical protein X777_09139, partial [Cerapachys biroi] 67 aa | EZA52127.1 GI: 607357681 |
| protein | |
| hypothetical protein X777_09138 [Cerapachys biroi] 403 aa | EZA52126.1 GI: 607357680 |
| protein | |
| hypothetical protein X777_09136 [Cerapachys biroi] 404 aa | EZA52125.1 GI: 607357679 |
| protein | |
| hypothetical protein X777_09135 [Cerapachys biroi] 404 aa | EZA52124.1 GI: 607357678 |
| protein | |
| hypothetical protein X777_09133 [Cerapachys biroi] 398 aa | EZA52123.1 GI: 607357677 |
| protein | |
| hypothetical protein X777_09132 [Cerapachys biroi] 408 aa | EZA52122.1 GI: 607357676 |
| protein | |
| hypothetical protein X777_09131 [Cerapachys biroi] 410 aa | EZA52121.1 GI: 607357675 |
| protein | |
| hypothetical protein X777_09130 [Cerapachys biroi] 410 aa | EZA52120.1 GI: 607357674 |
| protein | |
| hypothetical protein X777_09129 [Cerapachys biroi] 410 aa | EZA52119.1 GI: 607357673 |
| protein | |
| hypothetical protein X777_09127 [Cerapachys biroi] 413 aa | EZA52118.1 GI: 607357672 |
| protein | |
| hypothetical protein X777_09126 [Cerapachys biroi] 405 aa | EZA52117.1 GI: 607357671 |
| protein | |
| hypothetical protein X777_09124 [Cerapachys biroi] 402 aa | EZA52116.1 GI: 607357670 |
| protein | |
| hypothetical protein X777_09123 [Cerapachys biroi] 406 aa | EZA52115.1 GI: 607357669 |
| protein | |
| hypothetical protein X777_09298, partial [Cerapachys biroi] 316 | EZA52015.1 GI: 607357565 |
| aa protein | |
| hypothetical protein X777_08804 [Cerapachys biroi] 397 aa | EZA51620.1 GI: 607357154 |
| protein | |
| hypothetical protein X777_08803 [Cerapachys biroi] 395 aa | EZA51619.1 GI: 607357153 |
| protein | |
| hypothetical protein X777_08801 [Cerapachys biroi] 394 aa | EZA51618.1 GI: 607357152 |
| protein | |
| hypothetical protein X777_08800 [Cerapachys biroi] 395 aa | EZA51617.1 GI: 607357151 |
| protein | |
| hypothetical protein X777_08799 [Cerapachys biroi] 394 aa | EZA51616.1 GI: 607357150 |
| protein | |
| hypothetical protein X777_08798 [Cerapachys biroi] 394 aa | EZA51615.1 GI: 607357149 |
| protein | |
| hypothetical protein X777_08797 [Cerapachys biroi] 394 aa | EZA51614.1 GI: 607357148 |
| protein | |
| hypothetical protein X777_08795 [Cerapachys biroi] 397 aa | EZA51612.1 GI: 607357146 |
| protein | |
| hypothetical protein X777_08794 [Cerapachys biroi] 395 aa | EZA51611.1 GI: 607357145 |
| protein | |
| hypothetical protein X777_08793 [Cerapachys biroi] 353 aa | EZA51610.1 GI: 607357144 |
| protein | |
| hypothetical protein X777_08790 [Cerapachys biroi] 394 aa | EZA51607.1 GI: 607357141 |
| protein | |
| hypothetical protein X777_08789 [Cerapachys biroi] 394 aa | EZA51606.1 GI: 607357140 |
| protein | |
| hypothetical protein X777_08788 [Cerapachys biroi] 394 aa | EZA51605.1 GI: 607357139 |
| protein | |
| hypothetical protein X777_08787 [Cerapachys biroi] 390 aa | EZA51604.1 GI: 607357138 |
| protein | |
| hypothetical protein X777_08786 [Cerapachys biroi] 394 aa | EZA51603.1 GI: 607357137 |
| protein | |
| hypothetical protein X777_08785 [Cerapachys biroi] 394 aa | EZA51602.1 GI: 607357136 |
| protein | |
| hypothetical protein X777_08783, partial [Cerapachys biroi] 109 | EZA51600.1 GI: 607357134 |
| aa protein | |
| hypothetical protein X777_09662 [Cerapachys biroi] 379 aa | EZA51393.1 GI: 607356910 |
| protein | |
| hypothetical protein X777_10267, partial [Cerapachys biroi] 277 | EZA51181.1 GI: 607356681 |
| aa protein | |
| hypothetical protein X777_10879 [Cerapachys biroi] 350 aa | EZA50908.1 GI: 607356383 |
| protein | |
| hypothetical protein X777_10878 [Cerapachys biroi] 388 aa | EZA50907.1 GI: 607356382 |
| protein | |
| hypothetical protein X777_10877 [Cerapachys biroi] 393 aa | EZA50906.1 GI: 607356381 |
| protein | |
| hypothetical protein X777_10876 [Cerapachys biroi] 394 aa | EZA50905.1 GI: 607356380 |
| protein | |
| hypothetical protein X777_10874 [Cerapachys biroi] 393 aa | EZA50904.1 GI: 607356379 |
| protein | |
| hypothetical protein X777_10873 [Cerapachys biroi] 393 aa | EZA50903.1 GI: 607356378 |
| protein | |
| hypothetical protein X777_10872 [Cerapachys biroi] 394 aa | EZA50902.1 GI: 607356377 |
| protein | |
| hypothetical protein X777_10850 [Cerapachys biroi] 343 aa | EZA50881.1 GI: 607356356 |
| protein | |
| hypothetical protein X777_10849 [Cerapachys biroi] 331 aa | EZA50880.1 GI: 607356355 |
| protein | |
| hypothetical protein X777_10848 [Cerapachys biroi] 361 aa | EZA50879.1 GI: 607356354 |
| protein | |
| hypothetical protein X777_10661, partial [Cerapachys biroi] 286 | EZA50468.1 GI: 607355920 |
| aa protein | |
| hypothetical protein X777_10602 [Cerapachys biroi] 409 aa | EZA50409.1 GI: 607355861 |
| protein | |
| hypothetical protein X777_11257 [Cerapachys biroi] 393 aa | EZA50334.1 GI: 607355781 |
| protein | |
| hypothetical protein X777_11279 [Cerapachys biroi] 393 aa | EZA50278.1 GI: 607355719 |
| protein | |
| hypothetical protein X777_11081 [Cerapachys biroi] 365 aa | EZA50243.1 GI: 607355683 |
| protein | |
| hypothetical protein X777_11573 [Cerapachys biroi] 368 aa | EZA50143.1 GI: 607355570 |
| protein | |
| hypothetical protein X777_11736 [Cerapachys biroi] 398 aa | EZA50071.1 GI: 607355491 |
| protein | |
| hypothetical protein X777_11513 [Cerapachys biroi] 402 aa | EZA50024.1 GI: 607355443 |
| protein | |
| hypothetical protein X777_11512 [Cerapachys biroi] 373 aa | EZA50023.1 GI: 607355442 |
| protein | |
| hypothetical protein X777_11511 [Cerapachys biroi] 399 aa | EZA50022.1 GI: 607355441 |
| protein | |
| hypothetical protein X777_11510 [Cerapachys biroi] 400 aa | EZA50021.1 GI: 607355440 |
| protein | |
| hypothetical protein X777_11509 [Cerapachys biroi] 422 aa | EZA50020.1 GI: 607355439 |
| protein | |
| hypothetical protein X777_11508 [Cerapachys biroi] 410 aa | EZA50019.1 GI: 607355438 |
| protein | |
| hypothetical protein X777_11507 [Cerapachys biroi] 410 aa | EZA50018.1 GI: 607355437 |
| protein | |
| hypothetical protein X777_11506 [Cerapachys biroi] 405 aa | EZA50017.1 GI: 607355436 |
| protein | |
| hypothetical protein X777_l 1880 [Cerapachys biroi] 394 aa | EZA49382.1 GI: 607354770 |
| protein | |
| hypothetical protein X777_11877 [Cerapachys biroi] 400 aa | EZA49381.1 GI: 607354769 |
| protein | |
| hypothetical protein X777_12371 [Cerapachys biroi] 478 aa | EZA49341.1 GI: 607354726 |
| protein | |
| hypothetical protein X777_12369, partial [Cerapachys biroi] 376 | EZA49339.1 GI: 607354724 |
| aa protein | |
| hypothetical protein X777_12360 [Cerapachys biroi] 391 aa | EZA49330.1 GI: 607354715 |
| protein | |
| hypothetical protein X777_12504 [Cerapachys biroi] 391 aa | EZA49105.1 GI: 607354473 |
| protein | |
| hypothetical protein X777_12787 [Cerapachys biroi] 379 aa | EZA49075.1 GI: 607354438 |
| protein | |
| hypothetical protein X777_12783 [Cerapachys biroi] 352 aa | EZA49074.1 GI: 607354437 |
| protein | |
| hypothetical protein X777_12649, partial [Cerapachys biroi] 108 | EZA49046.1 GI: 607354404 |
| aa protein | |
| hypothetical protein X777_12820 [Cerapachys biroi] 366 aa | EZA49011.1 GI: 607354363 |
| protein | |
| hypothetical protein X777_12819 [Cerapachys biroi] 392 aa | EZA49010.1 GI: 607354362 |
| protein | |
| hypothetical protein X777_12817 [Cerapachys biroi] 404 aa | EZA49009.1 GI: 607354361 |
| protein | |
| hypothetical protein X777_12816 [Cerapachys biroi] 395 aa | EZA49008.1 GI: 607354360 |
| protein | |
| hypothetical protein X777_12814 [Cerapachys biroi] 402 aa | EZA49006.1 GI: 607354358 |
| protein | |
| hypothetical protein X777_12813 [Cerapachys biroi] 403 aa | EZA49005.1 GI: 607354357 |
| protein | |
| hypothetical protein X777_12906 [Cerapachys biroi] 393 aa | EZA48943.1 GI: 607354289 |
| protein | |
| hypothetical protein X777_12905 [Cerapachys biroi] 395 aa | EZA48942.1 GI: 607354288 |
| protein | |
| hypothetical protein X777_12904 [Cerapachys biroi] 393 aa | EZA48941.1 GI: 607354287 |
| protein | |
| hypothetical protein X777_12903 [Cerapachys biroi] 393 aa | EZA48940.1 GI: 607354286 |
| protein | |
| hypothetical protein X777_12902 [Cerapachys biroi] 393 aa | EZA48939.1 GI: 607354285 |
| protein | |
| hypothetical protein X777_12901 [Cerapachys biroi] 395 aa | EZA48938.1 GI: 607354284 |
| protein | |
| hypothetical protein X777_12900 [Cerapachys biroi] 394 aa | EZA48937.1 GI: 607354283 |
| protein | |
| hypothetical protein X777_12898 [Cerapachys biroi] 394 aa | EZA48935.1 GI: 607354281 |
| protein | |
| hypothetical protein X777_12897 [Cerapachys biroi] 394 aa | EZA48934.1 GI: 607354280 |
| protein | |
| hypothetical protein X777_12960 [Cerapachys biroi] 391 aa | EZA48918.1 GI: 607354259 |
| protein | |
| hypothetical protein X777_14095 [Cerapachys biroi] 393 aa | EZA48295.1 GI: 607353550 |
| protein | |
| hypothetical protein X777_14075 [Cerapachys biroi] 403 aa | EZA48275.1 GI: 607353530 |
| protein | |
| hypothetical protein X777_14074 [Cerapachys biroi] 395 aa | EZA48274.1 GI: 607353529 |
| protein | |
| hypothetical protein X777_14073 [Cerapachys biroi] 400 aa | EZA48273.1 GI: 607353528 |
| protein | |
| hypothetical protein X777_14071 [Cerapachys biroi] 398 aa | EZA48271.1 GI: 607353526 |
| protein | |
| hypothetical protein X777_14069 [Cerapachys biroi] 395 aa | EZA48269.1 GI: 607353524 |
| protein | |
| hypothetical protein X777_14157 [Cerapachys biroi] 393 aa | EZA48257.1 GI: 607353504 |
| protein | |
| hypothetical protein X777_14156 [Cerapachys biroi] 393 aa | EZA48256.1 GI: 607353503 |
| protein | |
| hypothetical protein X777_14154 [Cerapachys biroi] 294 aa | EZA48254.1 GI: 607353501 |
| protein | |
| hypothetical protein X777_14153 [Cerapachys biroi] 393 aa | EZA48253.1 GI: 607353500 |
| protein | |
| hypothetical protein X777_14152 [Cerapachys biroi] 390 aa | EZA48252.1 GI: 607353499 |
| protein | |
| hypothetical protein X777_14151 [Cerapachys biroi] 393 aa | EZA48251.1 GI: 607353498 |
| protein | |
| hypothetical protein X777_14150 [Cerapachys biroi] 393 aa | EZA48250.1 GI: 607353497 |
| protein | |
| hypothetical protein X777_14149 [Cerapachys biroi] 390 aa | EZA48249.1 GI: 607353496 |
| protein | |
| hypothetical protein X777_14148 [Cerapachys biroi] 422 aa | EZA48248.1 GI: 607353495 |
| protein | |
| hypothetical protein X777_14146 [Cerapachys biroi] 397 aa | EZA48247.1 GI: 607353494 |
| protein | |
| hypothetical protein X777_14145 [Cerapachys biroi] 397 aa | EZA48246.1 GI: 607353493 |
| protein | |
| hypothetical protein X777_14144 [Cerapachys biroi] 400 aa | EZA48245.1 GI: 607353492 |
| protein | |
| hypothetical protein X777_14142 [Cerapachys biroi] 392 aa | EZA48243.1 GI: 607353490 |
| protein | |
| hypothetical protein X777_14137 [Cerapachys biroi] 395 aa | EZA48239.1 GI: 607353486 |
| protein | |
| hypothetical protein X777_14135 [Cerapachys biroi] 370 aa | EZA48238.1 GI: 607353485 |
| protein | |
| hypothetical protein X777_14134 [Cerapachys biroi] 393 aa | EZA48237.1 GI: 607353484 |
| protein | |
| hypothetical protein X777_14133 [Cerapachys biroi] 393 aa | EZA48236.1 GI: 607353483 |
| protein | |
| hypothetical protein X777_14132 [Cerapachys biroi] 393 aa | EZA48235.1 GI: 607353482 |
| protein | |
| hypothetical protein X777_14128 [Cerapachys biroi] 393 aa | EZA48232.1 GI: 607353479 |
| protein | |
| hypothetical protein X777_14127 [Cerapachys biroi] 392 aa | EZA48231.1 GI: 607353478 |
| protein | |
| hypothetical protein X777_14238, partial [Cerapachys biroi] 110 | EZA48207.1 GI: 607353449 |
| aa protein | |
| hypothetical protein X777_14325 [Cerapachys biroi] 391 aa | EZA48143.1 GI: 607353377 |
| protein | |
| hypothetical protein X777_14324 [Cerapachys biroi] 391 aa | EZA48142.1 GI: 607353376 |
| protein | |
| hypothetical protein X777_14322 [Cerapachys biroi] 391 aa | EZA48140.1 GI: 607353374 |
| protein | |
| hypothetical protein X777_14321 [Cerapachys biroi] 332 aa | EZA48139.1 GI: 607353373 |
| protein | |
| hypothetical protein X777_14320 [Cerapachys biroi] 319 aa | EZA48138.1 GI: 607353372 |
| protein | |
| hypothetical protein X777_14319 [Cerapachys biroi] 394 aa | EZA48137.1 GI: 607353371 |
| protein | |
| hypothetical protein X777_14318, partial [Cerapachys biroi] 245 | EZA48136.1 GI: 607353370 |
| aa protein | |
| hypothetical protein X777_14166 [Cerapachys biroi] 410 aa | EZA48057.1 GI: 607353283 |
| protein | |
| hypothetical protein X777_14448 [Cerapachys biroi] 397 aa | EZA48027.1 GI: 607353226 |
| protein | |
| hypothetical protein X777_14454, partial [Cerapachys biroi] 110 | EZA48023.1 GI: 607353220 |
| aa protein | |
| hypothetical protein X777_15046, partial [Cerapachys biroi] 347 | EZA47950.1 GI: 607353115 |
| aa protein | |
| hypothetical protein X777_15045 [Cerapachys biroi] 277 aa | EZA47949.1 GI: 607353114 |
| protein | |
| hypothetical protein X777_15044 [Cerapachys biroi] 395 aa | EZA47948.1 GI: 607353113 |
| protein | |
| hypothetical protein X777_14494 [Cerapachys biroi] 393 aa | EZA47923.1 GI: 607353084 |
| protein | |
| hypothetical protein X777_14493, partial [Cerapachys biroi] 338 | EZA47922.1 GI: 607353083 |
| aa protein | |
| hypothetical protein X777_14490 [Cerapachys biroi] 392 aa | EZA47921.1 GI: 607353082 |
| protein | |
| hypothetical protein X777_14489 [Cerapachys biroi] 391 aa | EZA47920.1 GI: 607353081 |
| protein | |
| hypothetical protein X777_14488 [Cerapachys biroi] 357 aa | EZA47919.1 GI: 607353080 |
| protein | |
| hypothetical protein X777_14486 [Cerapachys biroi] 405 aa | EZA47917.1 GI: 607353078 |
| protein | |
| hypothetical protein X777_14484, partial [Cerapachys biroi] 367 | EZA47916.1 GI: 607353077 |
| aa protein | |
| hypothetical protein X777_15215 [Cerapachys biroi] 400 aa | EZA47891.1 GI: 607353036 |
| protein | |
| hypothetical protein X777_15213 [Cerapachys biroi] 396 aa | EZA47890.1 GI: 607353035 |
| protein | |
| hypothetical protein X777_15211 [Cerapachys biroi] 400 aa | EZA47889.1 GI: 607353034 |
| protein | |
| hypothetical protein X777_15210 [Cerapachys biroi] 402 aa | EZA47888.1 GI: 607353033 |
| protein | |
| hypothetical protein X777_15208 [Cerapachys biroi] 395 aa | EZA47887.1 GI: 607353032 |
| protein | |
| hypothetical protein X777_15207 [Cerapachys biroi] 391 aa | EZA47886.1 GI: 607353031 |
| protein | |
| hypothetical protein X777_15257 [Cerapachys biroi] 367 aa | EZA47872.1 GI: 607353013 |
| protein | |
| hypothetical protein X777_15254, partial [Cerapachys biroi] 203 | EZA47870.1 GI: 607353011 |
| aa protein | |
| hypothetical protein X777_15516 [Cerapachys biroi] 392 aa | EZA47757.1 GI: 607352872 |
| protein | |
| hypothetical protein X777_16325, partial [Cerapachys biroi] 347 | EZA47405.1 GI: 607352456 |
| aa protein | |
| hypothetical protein X777_16550 [Cerapachys biroi] 399 aa | EZA47250.1 GI: 607352245 |
| protein | |
| hypothetical protein X777_16549, partial [Cerapachys biroi] 387 | EZA47249.1 GI: 607352244 |
| aa protein | |
| hypothetical protein X777_16545, partial [Cerapachys biroi] 387 | EZA47248.1 GI: 607352243 |
| aa protein | |
| hypothetical protein X777_16583, partial [Cerapachys biroi] 115 | EZA47246.1 GI: 607352234 |
| aa protein | |
| hypothetical protein X777_16640, partial [Cerapachys biroi] 381 | EZA47175.1 GI: 607352154 |
| aa protein | |
| hypothetical protein X777_16639 [Cerapachys biroi] 388 aa | EZA47174.1 GI: 607352153 |
| protein | |
| hypothetical protein X777_16686 [Cerapachys biroi] 390 aa | EZA47122.1 GI: 607352100 |
| protein | |
| hypothetical protein X777_16765, partial [Cerapachys biroi] 257 | EZA47082.1 GI: 607352037 |
| aa protein | |
| hypothetical protein X777_17009, partial [Cerapachys biroi] 290 | EZA47033.1 GI: 607351973 |
| aa protein | |
| hypothetical protein X777_17008 [Cerapachys biroi] 395 aa | EZA47032.1 GI: 607351972 |
| protein | |
| hypothetical protein X777_17007 [Cerapachys biroi] 395 aa | EZA47031.1 GI: 607351971 |
| protein | |
| hypothetical protein X777_17006 [Cerapachys biroi] 357 aa | EZA47030.1 GI: 607351970 |
| protein | |
| hypothetical protein X777_17005 [Cerapachys biroi] 394 aa | EZA47029.1 GI: 607351969 |
| protein | |
| hypothetical protein X777_17004 [Cerapachys biroi] 394 aa | EZA47028.1 GI: 607351968 |
| protein | |
| hypothetical protein X777_17037 [Cerapachys biroi] 392 aa | EZA47010.1 GI: 607351937 |
| protein | |
| hypothetical protein X777_16829 [Cerapachys biroi] 394 aa | EZA46975.1 GI: 607351833 |
| protein | |
| hypothetical protein X777_16828 [Cerapachys biroi] 377 aa | EZA46974.1 GI: 607351832 |
| protein | |
| hypothetical protein X777_16827 [Cerapachys biroi] 395 aa | EZA46973.1 GI: 607351831 |
| protein | |
| hypothetical protein X777_16826 [Cerapachys biroi] 396 aa | EZA46972.1 GI: 607351830 |
| protein | |
| hypothetical protein X777_16825 [Cerapachys biroi] 390 aa | EZA46971.1 GI: 607351829 |
| protein | |
| hypothetical protein X777_16823 [Cerapachys biroi] 390 aa | EZA46970.1 GI: 607351828 |
| protein | |
| hypothetical protein X777_16822 [Cerapachys biroi] 390 aa | EZA46969.1 GI: 607351827 |
| protein | |
| hypothetical protein X777_00574 [Cerapachys biroi] 394 aa | EZA46944.1 GI: 607351788 |
| protein | |
| hypothetical protein X777_00572, partial [Cerapachys biroi] 284 | EZA46933.1 GI: 607351766 |
| aa protein | |
| hypothetical protein X777_00683, partial [Cerapachys biroi] 390 | EZA46927.1 GI: 607351750 |
| aa protein | |
| hypothetical protein X777_01187 [Cerapachys biroi] 395 aa | EZA46822.1 GI: 607351544 |
| protein | |
| hypothetical protein X777_01186 [Cerapachys biroi] 237 aa | EZA46821.1 GI: 607351543 |
| protein | |
| hypothetical protein X777_01182 [Cerapachys biroi] 393 aa | EZA46818.1 GI: 607351530 |
| protein | |
| hypothetical protein X777_01496, partial [Cerapachys biroi] 310 | EZA46816.1 GI: 607351520 |
| aa protein | |
| hypothetical protein X777_01992, partial [Cerapachys biroi] 104 | EZA46773.1 GI: 607351376 |
| aa protein | |
| hypothetical protein X777_02013 [Cerapachys biroi] 391 aa | EZA46772.1 GI: 607351374 |
| protein | |
| hypothetical protein X777_02041 [Cerapachys biroi] 391 aa | EZA46770.1 GI: 607351360 |
| protein | |
| hypothetical protein X777_02040 [Cerapachys biroi] 399 aa | EZA46769.1 GI: 607351359 |
| protein | |
| hypothetical protein X777_02038 [Cerapachys biroi] 394 aa | EZA46768.1 GI: 607351358 |
| protein | |
| hypothetical protein X777_02037 [Cerapachys biroi] 395 aa | EZA46767.1 GI: 607351357 |
| protein | |
| hypothetical protein X777_02036 [Cerapachys biroi] 351 aa | EZA46766.1 GI: 607351356 |
| protein | |
| hypothetical protein X777_02035 [Cerapachys biroi] 391 aa | EZA46765.1 GI: 607351355 |
| protein | |
| hypothetical protein X777_02034 [Cerapachys biroi] 391 aa | EZA46764.1 GI: 607351354 |
| protein | |
| hypothetical protein X777_02033 [Cerapachys biroi] 395 aa | EZA46763.1 GI: 607351353 |
| protein | |
| hypothetical protein X777_02154, partial [Cerapachys biroi] 388 | EZA46750.1 GI: 607351325 |
| aa protein | |
| hypothetical protein X777_02182, partial [Cerapachys biroi] 115 | EZA46748.1 GI: 607351315 |
| aa protein | |
| hypothetical protein X777_02391 [Cerapachys biroi] 395 aa | EZA46731.1 GI: 607351243 |
| protein | |
| hypothetical protein X777_02373 [Cerapachys biroi] 392 aa | EZA46714.1 GI: 607351226 |
| protein | |
| hypothetical protein X777_02372 [Cerapachys biroi] 313 aa | EZA46713.1 GI: 607351225 |
| protein | |
| hypothetical protein X777_03732 [Cerapachys biroi] 243 aa | EZA46661.1 GI: 607350938 |
| protein | |
| hypothetical protein X777_04259, partial [Cerapachys biroi] 110 | EZA46601.1 GI: 607350634 |
| aa protein | |
| hypothetical protein X777_04309, partial [Cerapachys biroi] 70 aa | EZA46600.1 GI: 607350630 |
| protein | |
| hypothetical protein X777_00022, partial [Cerapachys biroi] 114 | EZA46571.1 GI: 607349175 |
| aa protein | |
| hypothetical protein X777_00149, partial [Cerapachys biroi] 64 aa | EZA46447.1 GI: 607348111 |
| protein | |
| hypothetical protein X777_00236, partial [Cerapachys biroi] 230 | EZA46364.1 GI: 607347729 |
| aa protein | |
| Odorant receptor Or1; AgOr1 417 aa protein | Q8WTE7.1 GI: 44888255 |
| Odorant receptor Or2; AgOr2 378 aa protein | Q8WTE6.1 GI: 44888254 |
| uncharacterized protein LOC107040665 [Diachasma alloeum] | XP_015116341.1 GI: 970881283 |
| 1186 aa protein | |
| uncharacterized protein LOC107038380 [Diachasma alloeum] | XP_015112930.1 GI: 970880645 |
| 1173 aa protein | |
| uncharacterized protein LOC107040682 [Diachasma alloeum] 417 | XP_015116364.1 GI: 970881287 |
| aa protein | |
| uncharacterized protein LOC107038389 [Diachasma alloeum] 770 | XP_015112957.1 GI: 970880649 |
| aa protein | |
| Sensory neuron membrane protein 1; Short = SNMP1Dmel 551 aa | Q9VDD3.2 GI: 74868468 |
| protein | |
| General odorant-binding protein lush; Flags: Precursor 153 aa | O02372.1 GI: 61214421 |
| protein | |
| uncharacterized protein LOC107171897, partial [Diuraphis noxia] | XP_015377642.1 GI: 985424240 |
| 135 aa protein | |
| uncharacterized protein LOC107171052, partial [Diuraphis noxia] | XP_015376771.1 GI: 985422638 |
| 131 aa protein | |
| uncharacterized protein LOC107166471 [Diuraphis noxia] 403 aa | XP_015370631.1 GI: 985410377 |
| protein | |
| uncharacterized protein LOC107167838 [Diuraphis noxia] 113 aa | XP_015372525.1 GI: 985386955 |
| protein | |
| uncharacterized protein LOC107047710 [Diachasma alloeum] 134 | XP_015125998.1 GI: 970918690 |
| aa protein | |
| uncharacterized protein LOC107046523 [Diachasma alloeum] 435 | XP_015124630.1 GI: 970916388 |
| aa protein | |
| uncharacterized protein LOC107045828 [Diachasma alloeum] 131 | XP_015123693.1 GI: 970914661 |
| aa protein | |
| uncharacterized protein LOC107045792 [Diachasma alloeum] 274 | XP_015123634.1 GI: 970914552 |
| aa protein | |
| uncharacterized protein LOC107045791 [Diachasma alloeum] 377 | XP_015123633.1 GI: 970914550 |
| aa protein | |
| uncharacterized protein LOC107045316 isoform X2 [Diachasma | XP_015123022.1 GI: 970913423 |
| alloeum] 352 aa protein | |
| uncharacterized protein LOC107044760 [Diachasma alloeum] 389 | XP_015122273.1 GI: 970912048 |
| aa protein | |
| uncharacterized protein LOC107043060 [Diachasma alloeum] 234 | XP_015119847.1 GI: 970907601 |
| aa protein | |
| uncharacterized protein LOC107041505 [Diachasma alloeum] 154 | XP_015117568.1 GI: 970903454 |
| aa protein | |
| uncharacterized protein LOC107041471 [Diachasma alloeum] 392 | XP_015117541.1 GI: 970903394 |
| aa protein | |
| uncharacterized protein LOC107041468 [Diachasma alloeum] 284 | XP_015117538.1 GI: 970903388 |
| aa protein | |
| uncharacterized protein LOC107041058 [Diachasma alloeum] 171 | XP_015116902.1 GI: 970902214 |
| aa protein | |
| uncharacterized protein LOC107041057 [Diachasma alloeum] 173 | XP_015116901.1 GI: 970902212 |
| aa protein | |
| uncharacterized protein LOC107040351 [Diachasma alloeum] 394 | XP_015115892.1 GI: 970900366 |
| aa protein | |
| uncharacterized protein LOC107040339 [Diachasma alloeum] 313 | XP_015115876.1 GI: 970900336 |
| aa-protein | |
| uncharacterized protein LOC107039160 isoform X3 [Diachasma | XP_015114121.1 GI: 970897155 |
| alloeum] 335 aa protein | |
| uncharacterized protein LOC107039160 isoform X2 [Diachasma | XP_015114120.1 GI: 970897153 |
| alloeum] 383 aa protein | |
| uncharacterized protein LOC107039160 isoform X1 [Diachasma | XP_015114119.1 GI: 970897151 |
| alloeum] 391 aa protein | |
| uncharacterized protein LOC107039144 isoform X2 [Diachasma | XP_015114102.1 GI: 970897120 |
| alloeum] 327 aa protein | |
| uncharacterized protein LOC107039144 isoform X1 [Diachasma | XP_015114101.1 GI: 970897118 |
| alloeum] 383 aa protein | |
| uncharacterized protein LOC107038269 [Diachasma alloeum] 398 | XP_015112765.1 GI: 970894635 |
| aa protein | |
| uncharacterized protein LOC107038076 [Diachasma alloeum] 416 | XP_015112442.1 GI: 970894044 |
| aa protein | |
| uncharacterized protein LOC107038024 [Diachasma alloeum] 115 | XP_015112374.1 GI: 970893921 |
| aa protein | |
| uncharacterized protein LOC107037155 [Diachasma alloeum] 144 | XP_015111030.1 GI: 970891453 |
| aa protein | |
| uncharacterized protein LOC107037131 [Diachasma alloeum] 393 | XP_015111001.1 GI: 970891401 |
| aa protein | |
| uncharacterized protein LOC107037008 [Diachasma alloeum] 428 | XP_015110801.1 GI: 970891030 |
| aa protein | |
| uncharacterized protein LOC107036995 [Diachasma alloeum] 399 | XP_015110788.1 GI: 970891006 |
| aa protein | |
| uncharacterized protein LOC107036721 [Diachasma alloeum] 203 | XP_015110342.1 GI: 970890185 |
| aa protein | |
| uncharacterized protein LOC107036608 [Diachasma alloeum] 126 | XP_015110155.1 GI: 970889843 |
| aa protein | |
| uncharacterized protein LOC107036569 [Diachasma alloeum] 322 | XP_015110092.1 GI: 970889731 |
| aa protein | |
| uncharacterized protein LOC107036562 [Diachasma alloeum] 245 | XP_015110086.1 GI: 970889719 |
| aa protein | |
| uncharacterized protein LOC107035960 [Diachasma alloeum] 410 | XP_015109114.1 GI: 970887963 |
| aa protein | |
| uncharacterized protein LOC107048689 [Diachasma alloeum] 172 | XP_015127489.1 GI: 970885577 |
| aa protein | |
| uncharacterized protein LOC107048383 [Diachasma alloeum] 221 | XP_015127016.1 GI: 970884731 |
| aa protein | |
| uncharacterized protein LOC107048083 [Diachasma alloeum] 255 | XP_015126543.1 GI: 970883864 |
| aa protein | |
| uncharacterized protein LOC107041464 [Diachasma alloeum] 339 | XP_015117536.1 GI: 970881499 |
| aa protein | |
| uncharacterized protein LOC107041401 isoform X1 [Diachasma | XP_015117436.1 GI: 970881482 |
| alloeum] 405 aa protein | |
| uncharacterized protein LOC107040856 [Diachasma alloeum] 386 | XP_015116611.1 GI: 970881336 |
| aa protein | |
| uncharacterized protein LOC107040848 [Diachasma alloeum] 391 | XP_015116602.1 GI: 970881334 |
| aa protein | |
| uncharacterized protein LOC106692217, partial [Halyomorpha | XP_014293607.1 GI: 939698834 |
| halys] 120 aa protein | |
| uncharacterized protein LOC106692125, partial [Halyomorpha | XP_014293519.1 GI: 939698671 |
| halys] 120 aa protein | |
| uncharacterized protein LOC106691728, partial [Halyomorpha | XP_014293070.1 GI: 939697820 |
| halys] 309 aa protein | |
| uncharacterized protein LOC106690974 [Halyomorpha halys] 381 | XP_014292085.1 GI: 939695932 |
| aa protein | |
| uncharacterized protein LOC106690972 [Halyomorpha halys] 334 | XP_014292082.1 GI: 939695928 |
| aa protein | |
| uncharacterized protein LOC106690969 [Halyomorpha halys] 280 | XP_014292080.1 GI: 939695923 |
| aa protein | |
| uncharacterized protein LOC106690968 isoform X2 | XP_014292079.1 GI: 939695921 |
| [Halyomorpha halys] 356 aa protein | |
| uncharacterized protein LOC106690968 isoform X1 | XP_014292078.1 GI: 939695919 |
| [Halyomorpha halys] 381 aa protein | |
| uncharacterized protein LOC106690056 [Halyomorpha halys] 419 | XP_014290808.1 GI: 939693496 |
| aa protein | |
| uncharacterized protein LOC106689927 isoform X2 | XP_014290638.1 GI: 939693169 |
| [Halyomorpha halys] 402 aa protein | |
| uncharacterized protein LOC106689927 isoform X1 | XP_014290637.1 GI: 939693167 |
| [Halyomorpha halys] 430 aa protein | |
| uncharacterized protein LOC106689925 [Halyomorpha halys] 430 | XP_014290634.1 GI: 939693163 |
| aa protein | |
| uncharacterized protein LOC106689759 [Halyomorpha halys] 126 | XP_014290404.1 GI: 939692739 |
| aa protein | |
| uncharacterized protein LOC106689626, partial [Halyomorpha | XP_014290195.1 GI: 939692350 |
| halys] 361 aa protein | |
| uncharacterized protein LOC106689110 [Halyomorpha halys] 395 | XP_014289383.1 GI: 939690780 |
| aa protein | |
| uncharacterized protein LOC106689027, partial [Halyomorpha | XP_014289256.1 GI: 939690551 |
| halys] 349 aa protein | |
| uncharacterized protein LOC106688861 [Halyomorpha halys] 354 | XP_014289023.1 GI: 939690122 |
| aa protein | |
| uncharacterized protein LOC106688860 [Halyomorpha halys] 402 | XP_014289021.1 GI: 939690120 |
| aa protein | |
| uncharacterized protein LOC106688858 [Halyomorpha halys] 401 | XP_014289019.1 GI: 939690116 |
| aa protein | |
| uncharacterized protein LOC106688856 isoform X3 | XP_014289018.1 GI: 939690114 |
| [Halyomorpha halys] 291 aa protein | |
| uncharacterized protein LOC106688856 isoform X3 | XP_014289017.1 GI: 939690112 |
| [Halyomorpha halys] 291 aa protein | |
| uncharacterized protein LOC106688856 isoform X2 | XP_014289016.1 GI: 939690110 |
| [Halyomorpha halys] 349 aa protein | |
| uncharacterized protein LOC106688856 isoform X1 | XP_014289015.1 GI: 939690108 |
| [Halyomorpha halys] 391 aa protein | |
| uncharacterized protein LOC106688855 [Halyomorpha halys] 122 | XP_014289014.1 GI: 939690106 |
| aa protein | |
| uncharacterized protein LOC106688854 [Halyomorpha halys] 355 | XP_014289013.1 GI: 939690104 |
| aa protein | |
| uncharacterized protein LOC106688852 [Halyomorpha halys] 402 | XP_014289011.1 GI: 939690100 |
| aa protein | |
| uncharacterized protein LOC106688565 [Halyomorpha halys] 399 | XP_014288559.1 GI: 939689255 |
| aa protein | |
| uncharacterized protein LOC106688504 [Halyomorpha halys] 415 | XP_014288483.1 GI: 939689114 |
| aa protein | |
| uncharacterized protein LOC106688133 isoform X2 | XP_014287937.1 GI: 939688097 |
| [Halyomorpha halys] 264 aa protein | |
| uncharacterized protein LOC106688133 isoform X1 | XP_014287936.1 GI: 939688095 |
| [Halyomorpha halys] 299 aa protein | |
| uncharacterized protein LOC106687951 [Halyomorpha halys] 205 | XP_014287635.1 GI: 939687191 |
| aa protein | |
| uncharacterized protein LOC106687745 [Halyomorpha halys] 117 | XP_014287267.1 GI: 939686093 |
| aa protein | |
| uncharacterized protein LOC106687729 [Halyomorpha halys] 450 | XP_014287248.1 GI: 939686031 |
| aa protein | |
| uncharacterized protein LOC106687584 [Halyomorpha halys] 355 | XP_014287044.1 GI: 939685359 |
| aa protein | |
| uncharacterized protein LOC106687583 [Halyomorpha halys] 254 | XP_014287043.1 GI: 939685355 |
| aa protein | |
| uncharacterized protein LOC106687100 [Halyomorpha halys] 438 | XP_014286277.1 GI: 939683074 |
| aa protein | |
| uncharacterized protein LOC106686230 [Halyomorpha halys] 410 | XP_014284895.1 GI: 939678679 |
| aa protein | |
| uncharacterized protein LOC106686225 isoform X2 | XP_014284891.1 GI: 939678662 |
| [Halyomorpha halys] 382 aa protein | |
| uncharacterized protein LOC106686225 isoform X1 | XP_014284890.1 GI: 939678658 |
| [Halyomorpha halys] 419 aa protein | |
| uncharacterized protein LOC106684746 isoform X2 | XP_014282485.1 GI: 939671752 |
| [Halyomorpha halys] 235 aa protein | |
| uncharacterized protein LOC106684678 isoform X2 | XP_014282387.1 GI: 939671481 |
| [Halyomorpha halys] 140 aa protein | |
| uncharacterized protein LOC106684574 [Halyomorpha halys] 142 | XP_014282214.1 GI: 939671038 |
| aa protein | |
| uncharacterized protein LOC106684269 [Halyomorpha halys] 211 | XP_014281731.1 GI: 939669764 |
| aa protein | |
| uncharacterized protein LOC106682571 [Halyomorpha halys] 373 | XP_014278976.1 GI: 939662570 |
| aa protein | |
| uncharacterized protein LOC106682449 isoform X2 | XP_014278794.1 GI: 939662115 |
| [Halyomorpha halys] 322 aa protein | |
| uncharacterized protein LOC106682449 isoform X1 | XP_014278793.1 GI: 939662112 |
| [Halyomorpha halys] 323 aa protein | |
| uncharacterized protein LOC106682407, partial [Halyomorpha | XP_014278714.1 GI: 939661920 |
| halys] 265 aa protein | |
| uncharacterized protein LOC106681266 [Halyomorpha halys] 432 | XP_014276987.1 GI: 939657430 |
| aa protein | |
| uncharacterized protein LOC106681101, partial [Halyomorpha | XP_014276742.1 GI: 939656824 |
| halys] 182 aa protein | |
| uncharacterized protein LOC106681099 [Halyomorpha halys] 323 | XP_014276740.1 GI: 939656818 |
| aa protein | |
| uncharacterized protein LOC106680703 [Halyomorpha halys] 228 | XP_014276067.1 GI: 939654990 |
| aa protein | |
| uncharacterized protein LOC106680013 [Halyomorpha halys] 197 | XP_014274955.1 GI: 939651857 |
| aa protein | |
| uncharacterized protein LOC106679982 [Halyomorpha halys] 398 | XP_014274899.1 GI: 939651709 |
| aa protein | |
| uncharacterized protein LOC106679017 isoform X3 | XP_014273409.1 GI: 939647459 |
| [Halyomorpha halys] 392 aa protein | |
| uncharacterized protein LOC106679017 isoform X2 | XP_014273408.1 GI: 939647457 |
| [Halyomorpha halys] 402 aa protein | |
| uncharacterized protein LOC106679017 isoform X1 | XP_014273407.1 GI: 939647455 |
| [Halyomorpha halys] 427 aa protein | |
| uncharacterized protein LOC106678921 isoform X2 | XP_014273270.1 GI: 939647059 |
| [Halyomorpha halys] 385 aa protein | |
| uncharacterized protein LOC106678921 isoform X1 | XP_014273269.1 GI: 939647056 |
| [Halyomorpha halys] 437 aa protein | |
| uncharacterized protein LOC106678586 [Halyomorpha halys] 317 | XP_014272658.1 GI: 939645318 |
| aa protein | |
| uncharacterized protein LOC106678578 [Halyomorpha halys] 169 | XP_014272643.1 GI: 939645272 |
| aa protein | |
| uncharacterized protein LOC106678240 [Halyomorpha halys] 388 | XP_014272140.1 GI: 939643797 |
| aa protein | |
| uncharacterized protein LOC106677363 isoform X2 | XP_014270733.1 GI: 939639742 |
| [Halyomorpha halys] 417 aa protein | |
| uncharacterized protein LOC106677363 isoform X1 | XP_014270732.1 GI: 939639738 |
| [Halyomorpha halys] 419 aa protein | |
| uncharacterized protein LOC106677357 isoform X2 | XP_014270726.1 GI: 939639710 |
| [Halyomorpha halys] 370 aa protein | |
| uncharacterized protein LOC106677357 isoform X1 | XP_014270725.1 GI: 939639708 |
| [Halyomorpha halys] 404 aa protein | |
| uncharacterized protein LOC106677356 [Halyomorpha halys] 355 | XP_014270724.1 GI: 939639706 |
| aa protein | |
| uncharacterized protein LOC106693012 [Halyomorpha halys] 415 | XP_014294797.1 GI: 939638057 |
| aa protein | |
| uncharacterized protein LOC106693004 [Halyomorpha halys] 297 | XP_014294789.1 GI: 939638041 |
| aa protein | |
| uncharacterized protein LOC106692786 isoform X2 | XP_014294440.1 GI: 939637036 |
| [Halyomorpha halys] 348 aa protein | |
| uncharacterized protein LOC106692425 isoform X2 | XP_014293860.1 GI: 939635393 |
| [Halyomorpha halys] 378 aa protein | |
| uncharacterized protein LOC106687938 [Halyomorpha halys] 358 | XP_014287615.1 GI: 939633287 |
| aa protein | |
| uncharacterized protein LOC106684029 isoform X1 | XP_014281345.1 GI: 939631378 |
| [Halyomorpha halys] 400 aa protein | |
| uncharacterized protein LOC106683667 isoform X2 | XP_014280777.1 GI: 939631233 |
| [Halyomorpha halys] 345 aa protein | |
| uncharacterized protein LOC106683640 [Halyomorpha halys] 353 | XP_014280732.1 GI: 939631224 |
| aa protein | |
| uncharacterized protein LOC106683586, partial [Halyomorpha | XP_014280634.1 GI: 939631202 |
| halys] 400 aa protein | |
| uncharacterized protein LOC106681860 [Halyomorpha halys] 402 | XP_014277890.1 GI: 939630417 |
| aa protein | |
| uncharacterized protein LOC106681850 [Halyomorpha halys] 358 | XP_014277878.1 GI: 939630413 |
| aa protein | |
| uncharacterized protein LOC106681777 [Halyomorpha halys] 428 | XP_014277766.1 GI: 939630367 |
| aa protein | |
| uncharacterized protein LOC106678083 [Halyomorpha halys] 426 | XP_014271872.1 GI: 939628624 |
| aa protein | |
| uncharacterized protein LOC106692148 [Halyomorpha halys] 372 | XP_014293574.1 GI: 939627857 |
| aa protein | |
| olfactory receptor 3 [Bombyx mori] 439 aa protein | NP_001036925.1 GI: 112982950 |
| olfactory receptor 9 [Plutella xylostella] 449 aa protein | ALV82554.1 GI: 971834990 |
| olfactory receptor 2 [Bombyx mori] 472 aa protein | NP_001037060.1 GI: 112983084 |
| olfactory receptor 1 [Bombyx mori] 430 aa protein | NP_001036875.1 GI: 1 12983558 |
| Chain A, Structure Of Pheromone-binding Protein 1 In Complex | 4INX_A GI: 459358923 |
| With (z,z)-11,13-Hexadecadienol 140 aa protein | |
| Chain A, Structure Of Pheromone-binding Protein 1 In Complex | 4INW_A GI: 459358922 |
| With (11z,13z)-Hexadecadienal 140 aa protein | |
| olfactory receptor [Ostrinia fumacalis] 424 aa protein | BAH57982.1 GI: 229365469 |
| olfactory receptor [Ostrinia latipennis] 424 aa protein | BAH57981.1 GI: 229365467 |
| olfactory receptor [Ostrinia nubilalis] 424 aa protein | BAH57980.1 GI: 229365465 |
| olfactory receptor [Ostrinia ovalipennis] 424 aa protein | BAH57979.1 GI: 229365463 |
| olfactory receptor [Ostrinia palustralis] 424 aa protein | BAH57978.1 GI: 229365461 |
| olfactory receptor [Ostrinia zealis] 424 aa protein | BAH57977.1 GI: 229365459 |
| olfactory receptor [Ostrinia zaguliaevi] 424 aa protein | BAH57976.1 GI: 229365457 |
| olfactory receptor [Ostrinia scapulalis] 424 aa protein | BAH57975.1 GI: 229365455 |
| Sequence 10 from patent U.S. Pat. No. 7,601,829 486 aa protein | ADA08702.1 GI: 281014387 |
| Sequence 6 from patent U.S. Pat. No. 7,601,829 478 aa protein | ADA08700.1 GI: 281014385 |
| Sequence 4 from patent U.S. Pat. No. 7,601,829 472 aa protein | ADA08699.1 GI: 281014384 |
| Sequence 2 from patent U.S. Pat. No. 7,601,829 473 aa protein | ADA08698.1 GI: 281014383 |
| Sequence 10 from patent U.S. Pat. No. 7,550,574 486 aa protein | ACW03545.1 GI: 259184438 |
| Sequence 6 from patent U.S. Pat. No. 7,550,574 478 aa protein | ACW03543.1 GI: 259184436 |
| Sequence 4 from patent U.S. Pat. No. 7,550,574 472 aa protein | ACW03542.1 GI: 259184435 |
| Sequence 2 from patent U.S. Pat. No. 7,550,574 473 aa protein | ACW03541.1 GI: 259184434 |
| putative chemosensory receptor 2 [Antheraea pernyi] 472 aa | CAD88205.1 GI: 32399809 |
| protein | |
| hypothetical protein TcasGA2_TC032780 [Tribolium castaneum] | KYB27892.1 GI: 1004400598 |
| 1096 aa protein | |
| olfactory receptor 65 [Bombyx mori] 239 aa protein | NP_001166622.1 GI: 290560867 |
| olfactory receptor 13 [Bombyx mori] 385 aa protein | NP_001166603.1 GI: 290559921 |
| olfactory receptor-like [Bombyx mori] 410 aa protein | NP_001159623.1 GI: 261245107 |
| olfactory receptor 4 [Bombyx mori] 424 aa protein | NP_001036926.1 GI: 112982926 |
| AKH receptor variant AKHR3 isoform AKHR-B [Pseudoregma | AKH80290.1 GI: 822549471 |
| bambucicola] 591 aa protein | |
| olfactory receptor 10 [Bombyx mori] 388 aa protein | NP_001104819.1 GI: 162462631 |
| olfactory receptor 6 [Bombyx mori] 407 aa protein | NP_001036928.1 GI: 112982988 |
| olfactory receptor 5 [Bombyx mori] 417 aa protein | NP_001036927.1 GI: 112982948 |
| olfactory receptor 2 [Chilo suppressalis] 474 aa protein | AFQ94048.1 GI: 402746958 |
| unknown [Dendroctonus ponderosae] 382 aa protein | AEE63423.1 GI: 332376567 |
| unknown [Dendroctonus ponderosae] 396 aa protein | AEE63326.1 GI: 332376372 |
| unknown [Dendroctonus ponderosae] 404 aa protein | AEE63155.1 GI: 332376029 |
| unknown [Dendroctonus ponderosae] 394 aa protein | AEE62970.1 GI: 332375658 |
| unknown [Dendroctonus ponderosae] 395 aa protein | AEE62637.1 GI: 332374992 |
| unknown [Dendroctonus ponderosae] 400 aa protein | AEE62488.1 GI: 332374694 |
| unknown [Dendroctonus ponderosae] 480 aa protein | AEE62122.1 GI: 332373962 |
| unknown [Dendroctonus ponderosae] 396 aa protein | AEE61493.1 GI: 332372702 |
| unknown [Dendroctonus ponderosae] 403 aa protein | AEE61404.1 GI: 332372524 |
| olfactory receptor [Dendroctonus ponderosae] 480 aa protein | AFI45064.1 GI: 385200032 |
| putative olfactory receptor 18 [Spodoptera littoralis] 398 aa protein | ACL81189.1 GI: 220715234 |
| putative olfactory receptor 18 [Mamestra brassicae] 400 aa protein | ACL81188.1 GI: 220715232 |
| putative olfactory receptor 18 [Helicoverpa armigera] 398 aa | ACL81187.1 GI: 220715230 |
| protein | |
| putative olfactory receptor 18 [Helicoverpa zea] 398 aa protein | ACL81186.1 GI: 220715228 |
| putative olfactory receptor 18 [Agrotis segetum] 400 aa protein | ACL81185.1 GI: 220715226 |
| putative olfactory receptor 18 [Sesamia nonagrioides] 400 aa | ACL81184.1 GI: 220715224 |
| protein | |
| Sequence 18 from patent U.S. Pat. No. 7,601,829 33 aa protein | ADA08710.1 GI: 281014395 |
| Sequence 17 from patent U.S. Pat. No. 7,601,829 43 aa protein | ADA08709.1 GI: 281014394 |
| Sequence 16 from patent U.S. Pat. No. 7,601,829 43 aa protein | ADA08708.1 GI: 281014393 |
| Sequence 15 from patent U.S. Pat. No. 7,601,829 43 aa protein | ADA08707.1 GI: 281014392 |
| Sequence 14 from patent U.S. Pat. No. 7,601,829 43 aa protein | ADA08706.1 GI: 281014391 |
| Sequence 13 from patent U.S. Pat. No. 7,601,829 11 aa protein | ADA08705.1 GI: 281014390 |
| Sequence 12 from patent U.S. Pat. No. 7,601,829 16 aa protein | ADA08704.1 GI: 281014389 |
| Sequence 11 from patent U.S. Pat. No. 7,601,829 498 aa protein | ADA08703.1 GI: 281014388 |
| Sequence 8 from patent U.S. Pat. No. 7,601,829 486 aa protein | ADA08701.1 GI: 281014386 |
| Sequence 18 from patent U.S. Pat. No. 7,550,574 33 aa protein | ACW03553.1 GI: 259184446 |
| Sequence 17 from patent U.S. Pat. No. 7,550,574 43 aa protein | ACW03552.1 GI: 259184445 |
| Sequence 16 from patent U.S. Pat. No. 7,550,574 43 aa protein | ACW03551.1 GI: 259184444 |
| Sequence 15 from patent U.S. Pat. No. 7,550,574 43 aa protein | ACW03550.1 GI: 259184443 |
| Sequence 14 from patent U.S. Pat. No. 7,550,574 43 aa protein | ACW03549.1 GI: 259184442 |
| Sequence 13 from patent U.S. Pat. No. 7,550,574 11 aa protein | ACW03548.1 GI: 259184441 |
| Sequence 12 from patent U.S. Pat. No. 7,550,574 16 aa protein | ACW03547.1 GI: 259184440 |
| Sequence 11 from patent U.S. Pat. No. 7,550,574 498 aa protein | ACW03546.1 GI: 259184439 |
| Sequence 8 from patent U.S. Pat. No. 7,550,574 486 aa protein | ACW03544.1 GI: 259184437 |
| Sequence 6 from patent U.S. Pat. No. 7,541,155 486 aa protein | ACS10701.1 GI: 239686039 |
| Sequence 4 from patent U.S. Pat. No. 7,541,155 376 aa protein | ACS10700.1 GI: 239686038 |
| unnamed protein product [Drosophila melanogaster] 379 aa | CAY86014.1 GI: 237677885 |
| protein | |
| unnamed protein product [Drosophila melanogaster] 376 aa | CAY86011.1 GI: 237677879 |
| protein | |
| unnamed protein product [Drosophila melanogaster] 467 aa | CAY86010.1 GI: 237677877 |
| protein | |
| unnamed protein product, partial [Drosophila melanogaster] 153 aa | CAY86009.1 GI: 237677875 |
| protein | |
| Sequence 104 from patent U.S. Pat. No. 7,241,881 486 aa protein | ABU34893.1 GI: 155712034 |
| Sequence 100 from patent U.S. Pat. No. 7,241,881 392 aa protein | ABU34891.1 GI: 155712032 |
| Sequence 98 from patent U.S. Pat. No. 7,241,881 406 aa protein | ABU34890.1 GI: 155712031 |
| Sequence 78 from patent U.S. Pat. No. 7,241,881 378 aa protein | ABU34880.1 GI: 155712021 |
| Sequence 70 from patent U.S. Pat. No. 7,241,881 392 aa protein | ABU34876.1 GI: 155712017 |
| Sequence 68 from patent U.S. Pat. No. 7,241,881 397 aa protein | ABU34875.1 GI: 155712016 |
| Sequence 66 from patent U.S. Pat. No. 7,241,881 413 aa protein | ABU34874.1 GI: 155712015 |
| Sequence 50 from patent U.S. Pat. No. 7,241,881 398 aa protein | ABU34866.1 GI: 155712007 |
| Sequence 40 from patent U.S. Pat. No. 7,241,881 412 aa protein | ABU34861.1 GI: 155712002 |
| Sequence 34 from patent U.S. Pat. No. 7,241,881 383 aa protein | ABU34858.1 GI: 155711999 |
| Sequence 30 from patent U.S. Pat. No. 7,241,881 396 aa protein | ABU34856.1 GI: 155711997 |
| Sequence 28 from patent U.S. Pat. No. 7,241,881 375 aa protein | ABU34855.1 GI: 155711996 |
| Sequence 24 from patent U.S. Pat. No. 7,241,881 385 aa protein | ABU34853.1 GI: 155711994 |
| Sequence 20 from patent U.S. Pat. No. 7,241,881 379 aa protein | ABU34851.1 GI: 155711992 |
| Sequence 18 from patent U.S. Pat. No. 7,241,881 378 aa protein | ABU34850.1 GI: 155711991 |
| Sequence 16 from patent U.S. Pat. No. 7,241,881 379 aa protein | ABU34849.1 GI: 155711990 |
| Sequence 12 from patent U.S. Pat. No. 7,241,881 379 aa protein | ABU34847.1 GI: 155711988 |
| Sequence 10 from patent U.S. Pat. No. 7,241,881 397 aa protein | ABU34846.1 GI: 155711987 |
| Sequence 8 from patent U.S. Pat. No. 7,241,881 397 aa protein | ABU34845.1 GI: 155711986 |
| Sequence 6 from patent U.S. Pat. No. 7,241,881 376 aa protein | ABU34844.1 GI: 155711985 |
| Sequence 4 from patent U.S. Pat. No. 7,241,881 467 aa protein | ABU34843.1 GI: 155711984 |
| Sequence 2 from patent U.S. Pat. No. 7,241,881 397 aa protein | ABU34842.1 GI: 155711983 |
| olfactory receptor-like receptor [Bombyx mori] 407 aa protein | BAD89570.1 GI: 59796989 |
| olfactory receptor-like receptor [Bombyx mori] 417 aa protein | BAD89569.1 GI: 59796987 |
| olfactory receptor-like receptor [Bombyx mori] 424 aa protein | BAD89568.1 GI: 59796985 |
| olfactory receptor-like receptor [Bombyx mori] 439 aa protein | BAD89567.1 GI: 59796983 |
| putative chemosensory receptor 2, partial [Tenebrio molitor] 206 | CAD88247.1 GI: 32400236 |
| aa protein | |
| putative chemosensory receptor 2, partial [Calliphora vicina] 208 | CAD88246.1 GI: 32400234 |
| aa protein | |
| putative chemosensory receptor 2, partial [Apis mellifera] 210 aa | CAD88245.1 GI: 32399813 |
| protein | |
| putative chemosensory receptor 2 [Bombyx mori] 472 aa protein | CAD88206.1 GI: 32399811 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-78. (canceled)

79. A volatile compound detection system comprising:
an array having a plurality of chambers, a plurality of cells in each chamber, each cell genetically modified to express one or more odorant receptors having a binding specificity for a volatile compound, wherein binding of volatile compound to the receptor generates a luminescent signal in the genetically modified cell;
a life support system operatively connected to the array and configured to provide a liquid environment;
a gas delivery system configured to perfuse a cell culture media through the array;
wherein a volatile compound from an external environment dissolves in the liquid for binding to the one or more odorant receptors.

80. The volatile compound detection system of claim 79 wherein the volatile compound comprises TNT, DNT and/or RDX.

81. The volatile compound detection system of claim 79 wherein the volatile compound comprises vanillic acid.

82. The volatile compound detection system of claim 79 wherein the volatile compound comprises an illegal substance.

83. The volatile compound detection system of claim 79 wherein the volatile compound comprises a chemical weapon.

84. The volatile compound detection system of claim 79 wherein the volatile compound comprises a carcinogen.

85. The volatile compound detection system of claim 79 wherein the luminescence signal is proportional to the amount of volatile compound in an air sample.

86. The volatile compound detection system of claim 79 wherein the receptors are fine tuned.

87. The volatile compound detection system of claim 79 wherein the receptors are broad tuned.

88. The volatile compound detection system of claim 79 wherein the cells express one receptor.

89. The volatile compound detection system of claim 79 wherein the cells express multiple receptors.

90. The volatile compound detection system of claim 79 wherein the receptors bind to more than one volatile compound.

91. A volatile compound detection system comprising:
an array having a plurality of chambers, a plurality of cells in each chamber, the cells genetically modified to express one or more odorant receptors having a binding specificity for a volatile compound, wherein binding of the volatile compound to the odorant receptor generates an optically detectable signal in the genetically modified cell;
the array having at least one perfusion channel fluidically coupled to one or more of the plurality of chambers;
the array adapted to transport an air sample so that the genetically modified cells are exposed to the volatile compound through a membrane containing liquid media, the volatile compound passing through the membrane to the genetically modified cells;
an optical sensor associated with one or more of the chambers; and
a computer controller configured to receive a measurement from the optical sensor.

92. The volatile compound detection system of claim 91 wherein the volatile compound comprises an explosive.

93. The volatile compound detection system of claim 91 wherein the volatile compound comprises an illegal substance.

94. The volatile compound detection system of claim 91 wherein the volatile compound comprises a carcinogen.

95. The volatile compound detection system of claim 91 wherein the volatile compound comprises an explosive or a chemical weapon.

96. The volatile compound detection system of claim 91 wherein the receptors are tuned to improve detection sensitivity.

97. The volatile compound detection system of claim 91 wherein the cells express the same or different receptors.
